(19) United States
(12) Patent Application Publication
(10) Pub. No.: US 2014/0243809 A1
(43) Pub. Date: Aug. 28, 2014

(54) ENDOVASCULAR CATHETERS FOR TRANS-SUPERFICIAL TEMPORAL ARTERY TRANSMURAL CAROTID BODY MODULATION

(71) Applicants: Mark GELFAND, New York, NY (US); Howard LEVIN, Teaneck, NJ (US); Charles LENNOX, Hudson, NH (US); Marcus W. WILBORN, Huntsville, AL (US); Paul A. SOBOTKA, West St. Paul, MN (US); Zoar Jacob ENGELMAN, Salt Lake City, UT (US); Martin M. GRASSE, Boston, MA (US); Mark S. LEUNG, Duncan (CA)

(72) Inventors: Mark GELFAND, New York, NY (US); Howard LEVIN, Teaneck, NJ (US); Charles LENNOX, Hudson, NH (US); Marcus W. WILBORN, Huntsville, AL (US); Paul A. SOBOTKA, West St. Paul, MN (US); Zoar Jacob ENGELMAN, Salt Lake City, UT (US); Martin M. GRASSE, Boston, MA (US); Mark S. LEUNG, Duncan (CA)

(21) Appl. No.: 14/188,452

(22) Filed: Feb. 24, 2014

Related U.S. Application Data
(60) Provisional application No. 61/768,101, filed on Feb. 22, 2013.

(51) Int. Cl. A61B 18/14

(52) U.S. Cl. CPC A61B 18/1492; A61B 2018/00577
USPC 606/28; 606/41

(57) ABSTRACT

Methods, devices, and systems for carotid body modulation via accessing a target site with an endovascular approach through a superficial temporal artery.

### CROSS REFERENCE TO RELATED APPLICATIONS

This application claims the benefit of U.S. Prov. App. 61/768,101, filed Feb. 22, 2013, which application is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

The present disclosure is directed generally to devices, systems and methods for treating patients having sympathetically mediated disease associated at least in part with augmented peripheral chemoreflex, heightened sympathetic activation, or autonomic imbalance by ablating at least one peripheral chemoreceptor (e.g., carotid body) with an endovascular transmural ablation catheter configured for access to a carotid bifurcation or intercarotid septum, by means of trans-superficial temporal artery arterial access.

### BACKGROUND

It is known that an imbalance of the autonomic nervous system is associated with several disease states. Restoration of autonomic balance has been a target of several medical treatments including modalities such as pharmacological, device-based, and electrical stimulation. For example, beta blockers are a class of drugs used to reduce sympathetic activity to treat cardiac arrhythmias and hypertension; Gelfand and Levin (U.S. Pat. No. 7,162,303) describe a device-based treatment used to decrease renal sympathetic activity to treat heart failure, hypertension, and renal failure; Yun and Yuarn-Bor (U.S. Pat. No. 7,149,574; U.S. Pat. No. 7,363,076; U.S. Pat. No. 7,738,952) describe a method of restoring autonomic balance by increasing parasympathetic activity to treat disease associated with parasympathetic attrition; Kieval, Burns and Serdar (U.S. Pat. No. 8,060,206) describe an electrical pulse generator that stimulates a baroreceptor, increasing parasympathetic activity, in response to high blood pressure; Hlavka and Elliott (US 2010/0070004) describe an implantable electrical stimulator in communication with an afferent neural pathway of a carotid body chemoreceptor to control dyspnea via electrical neuromodulation. More recently, Carotid Body Modulation (CBM) also referred to as Carotid Body Ablation (CBA) has been conceived for treating sympathetically mediated diseases. Geometry of human vasculature is highly variable, including geometry of the aortic arch, and left and right carotid bifurcations. In some patients it may be difficult or traumatic to approach a target carotid bifurcation from the aorta (e.g., via femoral artery access). There is a need for devices, systems and methods for carotid body modulation via an alternative endovascular approach.

### SUMMARY

Methods, devices, and systems have been conceived for endovascular transmural ablation of a carotid body with a catheter configured for trans-superficial temporal artery access to the region of an intercarotid septum. Endovascular ablation of a carotid body generally refers to delivering a device through a patient's vasculature to a blood vessel proximate to a target ablation site (e.g., carotid body, intercarotid plexus, carotid body nerves) of the patient and placing an ablation element associated with the device proximate to the peripheral chemosensor in a configuration that directs ablative energy at the target ablation site and activating the ablation element to ablate the peripheral chemosensor. Trans-superficial temporal artery access refers to introducing an endovascular carotid body ablation catheter into a superficial temporal artery and delivering the catheter in a retrograde direction to the vicinity of the associated intercarotid septum for the purpose of ablating or modulating a function of a carotid body.

A carotid body may be ablated by placing an ablation element within and against the wall of a carotid artery adjacent to the carotid body of interest, then delivering ablation energy from the ablation element causing a change in temperature of periarterial space containing the carotid body to an extent and duration sufficient to ablate the carotid body.

Devices have been conceived that couple with a carotid bifurcation to facilitate orientation, positioning and apposition of one or more ablation elements at a target ablation site or sites suitable for carotid body modulation. The devices may be configured to measure tissue impedance across an intercarotid septum.

In another exemplary procedure a location of periarterial space associated with a carotid body is identified, then an ablation element is placed against the interior wall of a carotid artery adjacent to the identified location, then ablation parameters are selected and the ablation element is activated thereby ablating the carotid body, whereby the position of the ablation element and the selection of ablation parameters provides for ablation of the carotid body without substantial collateral damage to adjacent functional structures.

In further example the location of the periarterial space associated with a carotid body is identified, as well as the location of important non-target nerve structures not associated with the carotid body, then an ablation element is placed against the interior wall of a carotid artery adjacent to the identified location, ablation parameters are selected and the ablation element is then activated thereby ablating the carotid body, whereby the position of the ablation element and the selection of ablation parameters provides for ablation of the target site (e.g., carotid body, carotid body nerves, intercarotid septum) without substantial collateral damage to important non-target nerve structures in the vicinity of the carotid body. A device configured to prevent embolic debris from entering the brain may be deployed in an internal carotid artery associated with a carotid body, then an ablation element is placed within and against the wall of an external carotid artery or an internal carotid artery associated with the carotid body, the ablation element is activated resulting in carotid body modulation, the ablation element is then withdrawn, then the embolic prevention device is withdrawn, whereby the embolic prevention device in the internal carotid artery prevents debris resulting from the use of the ablation element form entering the brain.

A method has been conceived in which the location of the perivascular space associated with a carotid body is identified, then an ablation element is placed in a predetermined location against the interior wall of vessel adjacent to the identified location, then ablation parameters are selected and the ablation element is activated and then deactivated, the ablation element is then repositioned in at least one additional predetermine location against the same interior wall and the ablation element is then reactivated using the same or different ablation parameters, whereby the positions of the ablation element and the selection of ablation parameters provides for ablation of the carotid body without substantial collateral damage to adjacent functional structures.

A method has been conceived by which a location of perivascular space associated with a carotid body is identified, an ablation element configured for tissue freezing is placed against an interior wall of a vessel adjacent to the identified location, ablation parameters are selected for reversible cryo-ablation and the ablation element is activated, effectiveness of the ablation is then determined by at least one physiological response to the ablation, and if the determination is that the physiological response is favorable, then the ablation element is reactivated using the ablation parameters selected for permanent carotid body modulation.

A system has been conceived comprising a vascular catheter configured with an ablation element in the vicinity of the distal end, and a connection between the ablation element and a source of ablation energy at the proximal end, whereby the distal end of the catheter is constructed to be inserted into a superficial temporal artery, or another distal branch to an external carotid artery of a patient and then maneuvered into an internal or external carotid artery using standard fluoroscopic guidance techniques.

A system has been conceived comprising a vascular catheter configured for trans-superficial temporal arterial access with an ablation element in vicinity of a distal end configured for carotid body modulation and further configured for at least one of the following: neural stimulation, neural blockade, carotid body stimulation and carotid body blockade; and a connection between the ablation element and a source of ablation energy, stimulation energy and/or blockade energy.

A system has been conceived comprising a vascular catheter configured for trans-superficial temporal arterial access with an ablation element and at least one electrode configured for at least one of the following: neural stimulation, neural blockade, carotid body stimulation and carotid body blockade; and a connection between the ablation element to a source of ablation energy, and a connection between the ablation element and/or electrode(s) to a source of stimulation energy and/or blockade energy.

A system has been conceived comprising a vascular catheter configured for trans-superficial temporal arterial access with an ablation element mounted in the vicinity of a distal end configured for tissue heating, whereby, the ablation element comprises at least one electrode and at least one temperature sensor, a connection between the ablation element electrode(s) and temperature sensor(s) to an ablation energy source, with the ablation energy source being configured to maintain the ablation element at a temperature in the range of 36 to 100 degrees centigrade during ablation using signals received from the temperature sensor(s). For example, in an embodiment the at least one ablation element in contact with blood is maintained at a temperature between 36 and 50 degrees centigrade to minimize coagulation while targeted periarterial tissue is heated to a temperature between 50 and 100 degrees centigrade to ablate tissue but avoid boiling of water and steam and gas expansion in the tissue.

A system has been conceived comprising a vascular catheter configured for trans-superficial temporal arterial access with an ablation element mounted in vicinity of a distal end configured for tissue heating, whereby, the ablation element comprises at least one electrode and at least one temperature sensor and at least one irrigation channel, and a connection between the ablation element electrode(s) and temperature sensor(s) and irrigation channel(s) to an ablation energy source, with the ablation energy source being configured to maintain the ablation element at a temperature in the range of 36 to 100 degrees centigrade during ablation using signals received from the temperature sensor(s) and by providing irrigation to the vicinity of the ablation element. For example, in an embodiment the at least one ablation element in contact with blood is maintained at a temperature between 36 and 50 degrees centigrade to minimize coagulation while targeted periarterial tissue is heated to a temperature between 50 and 100 degrees centigrade to ablate tissue but avoid boiling of water and steam and gas expansion in the tissue.

A system has been conceived comprising a vascular catheter configured for trans-superficial temporal arterial access with an ablation element mounted in vicinity of a distal end configured for tissue freezing, whereby, the ablation element comprises at least one cryogenic expansion chamber and at least one temperature sensor, and a connection between the ablation element expansion chamber and temperature sensor(s) to a cryogenic agent source, with the cryogenic agent source being configured to maintain the ablation element at a predetermined temperature in the range of −20 to −160 degrees centigrade during ablation using signals received from the temperature sensor(s).

A system for endovascular transmural ablation of a carotid body has been conceived comprising a carotid artery catheter configured for trans-superficial temporal arterial access with an ablation element mounted on a distal region of the catheter, a means for pressing the ablation element against a wall of a carotid artery at a specific location, a means for connecting the ablation element to a source of ablation energy mounted at a proximal region of the catheter, and a console comprising a source of ablation energy, a means for controlling the ablation energy, a user interface configured to provide the user with a selection of ablation parameters, indications of the status of the console and the status of the ablation activity, a means to activate and deactivate an ablation, and an umbilical to provide a means for connecting the catheter to the console.

A method has been conceived to reduce or inhibit chemoreflex generated by a carotid body in a patient, to reduce afferent nerve sympathetic activity of carotid body nerves to treat a sympathetically mediated disease, the method comprising: inserting a catheter into a superficial temporal artery of the patient in the retrograde direction, positioning the catheter such that a distal section of the catheter is in the external carotid artery proximate to a carotid body of the patient; pressing an ablation element against the wall of an external carotid artery, and/or an internal carotid artery adjacent to the carotid body, supplying energy to the ablation element(s) wherein the energy is supplied by an energy supply apparatus outside of the patient; applying the energy from the energy supply to the ablation element(s) to ablate tissue proximate to or included in the carotid body; and removing the ablation device from the patient; wherein a carotid body chemoreflex function is inhibited or sympathetic afferent nerve activity of carotid body nerves is reduced due to the ablation.

A method has been conceived to treat a patient having a sympathetically mediated disease by reducing or inhibiting chemoreflex function generated by a carotid body including steps of inserting a catheter into a superficial temporal artery or another distal branch of an external carotid artery of a patient's vasculature, positioning a portion of the catheter proximate a carotid body (e.g., in a carotid artery, proximate an intercarotid septum), positioning an ablation element toward a target ablation site (e.g., carotid body, intercarotid septum, carotid plexus, carotid sinus nerve), holding position of the catheter, applying ablative energy to the target ablation site via the ablation element, and removing the catheter from the patient's vasculature.

A vascular catheter has been conceived for modulation of carotid body function in a patient comprising, a catheter shaft with a caliber between approximately 3 French and 6 French, with a working length between approximately 10 cm and 25 cm, at least one ablation element mounted in the vicinity of the distal end, a mechanism configured for positioning the ablation element(s) against the wall of an external carotid artery adjacent to a target site (e.g., a carotid body, carotid body nerves, an intercarotid septum), a means for providing the user with a substantially unambiguous fluoroscopic indication of the position of the ablation element(s) within the external carotid artery, and a means for connecting the ablation element to a source of ablation energy mounted in the vicinity of the proximal end.

A vascular catheter has been conceived for carotid body modulation in a patient comprising, a catheter shaft with a caliber between approximately 3 French and 6 French, with a working length between approximately 10 cm and 25 cm, an ablation element mounted in the vicinity of the distal end, a mechanism configured for positioning the ablation element against the wall of an external carotid artery adjacent to a carotid body, a means for providing the user with a substantially unambiguous fluoroscopic indication of the position of the ablation element within the external carotid artery, and a means for connecting the ablation element to a source of ablation energy mounted in the vicinity of the proximal end, whereby the ablation element is a cylindrical monopolar RF electrode, and the source of ablation energy is a radiofrequency energy generator configured for carotid body modulation.

A vascular catheter has been conceived for carotid body modulation in a patient comprising, a catheter shaft with a caliber between approximately 3 French and 6 French, with a working length between approximately 10 cm and 25 cm, an ablation element mounted in the vicinity of the distal end, a mechanism configured for positioning the ablation element against the wall of an external carotid artery adjacent to a carotid body, a means for providing the user with a substantially unambiguous fluoroscopic indication of the position of the ablation element within the external carotid artery, and a means for connecting the ablation element to a source of ablation energy mounted in the vicinity of the proximal end, whereby the ablation element is a lateral monopolar RF electrode configured to apply RF energy to the wall of an external carotid artery and avoid applying RF energy to arterial blood, and the source of ablation energy is a radiofrequency energy generator configured for carotid body modulation.

A vascular catheter has been conceived for carotid body modulation in a patient comprising, a catheter shaft with a caliber between approximately 3 French and 6 French, with a working length between approximately 10 cm and 25 cm, an ablation element mounted in the vicinity of the distal end, a mechanism configured for positioning the ablation element against the wall of an external carotid artery adjacent to a carotid body, a means for providing the user with a substantially unambiguous fluoroscopic indication of the position of the ablation element within the external carotid artery, a means for connecting the ablation element to a source of ablation energy mounted in the vicinity of the proximal end, and a means for connecting the ablation element to a source ionic liquid, whereby the ablation element is a cylindrical monopolar RF electrode with a means for substantial surface irrigation by ionic liquid, and the source of ablation energy is a radiofrequency energy generator configured for carotid body modulation.

A vascular catheter has been conceived for carotid body modulation in a patient comprising, a catheter shaft with a caliber between approximately 3 French and 6 French, with a working length between approximately 10 cm and 25 cm, an ablation element mounted in the vicinity of the distal end, a mechanism configured for positioning the ablation element against the wall of an external carotid artery adjacent to a carotid body, a means for providing the user with a substantially unambiguous fluoroscopic indication of the position of the ablation element within the external carotid artery, a means for connecting the ablation element to a source of ablation energy mounted in the vicinity of the proximal end, and a means for connecting the ablation element to a source ionic liquid, whereby the ablation element is a lateral monopolar RF electrode configured to apply RF energy to the wall of an external carotid artery and avoid applying RF energy to arterial blood, with a means for substantial surface irrigation by ionic liquid, where the source of ablation energy is a radiofrequency energy generator configured for carotid body modulation.

A vascular catheter has been conceived for carotid body modulation in a patient comprising, a catheter shaft with a caliber between approximately 3 French and 6 French, with a working length between approximately 10 cm and 25 cm, an ablation element mounted in the vicinity of the distal end, a mechanism configured for positioning the ablation element against the wall of an external carotid artery adjacent to a carotid body, a means for providing the user with a substantially unambiguous fluoroscopic indication of the position of the ablation element within the external carotid artery, a means for connecting the ablation element to a source of ablation energy mounted in the vicinity of the proximal end, and a means for connecting the ablation element to a source ionic liquid, whereby the ablation element comprises a hollow cylindrical structure with at least one lateral fenestration, at least one lumen within the catheter shaft in communication with the interior of the hollow cylindrical structure and the fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface, and the source of ablation energy is a radiofrequency energy generator configured for carotid body modulation.

A vascular catheter has been conceived for carotid body modulation in a patient comprising, a catheter shaft with a caliber between approximately 3 French and 6 French, with a working length between approximately 10 cm and 25 cm, an ablation element mounted in the vicinity of the distal end, a mechanism configured for positioning the ablation element against the wall of an external carotid artery adjacent to a carotid body, a means for providing the user with a substantially unambiguous fluoroscopic indication of the position of the ablation element within the external carotid artery, and a means for connecting the ablation element to a source of ablation energy mounted in the vicinity of the proximal end, whereby the ablation element is a bipolar pair of RF electrodes mounted in tandem, with each electrode connectable to an opposite pole of a radiofrequency energy generator configured for carotid body modulation.

A vascular catheter has been conceived for carotid body modulation in a patient comprising, a catheter shaft with a caliber between approximately 3 French and 6 French, with a working length between approximately 10 cm and 25 cm, an ablation element mounted in the vicinity of the distal end, a mechanism configured for positioning the ablation element against the wall of an external carotid artery adjacent to a carotid body, a means for providing the user with a substantially unambiguous fluoroscopic indication of the position of the ablation element within the external carotid artery, and a means for connecting the ablation element to a source of ablation energy mounted in the vicinity of the proximal end, whereby the ablation element is a lateral bipolar pair of RF electrodes mounted in tandem configured to apply RF energy to the wall of an external carotid artery and minimize applying RF energy to arterial blood, with each electrode connectable to an opposite pole of a radiofrequency energy generator configured for carotid body modulation.

A vascular catheter has been conceived for carotid body modulation in a patient comprising, a catheter shaft with a caliber between approximately 3 French and 6 French, with a working length between approximately 10 cm and 25 cm, an ablation element mounted in the vicinity of the distal end, a mechanism configured for positioning the ablation element against the wall of an external carotid artery adjacent to a carotid body, a means for providing the user with a substantially unambiguous fluoroscopic indication of the position of the ablation element within the external carotid artery, and a means for connecting the ablation element to a source of ablation energy mounted in the vicinity of the proximal end, and a means for connecting the ablation element to a source ionic liquid, whereby the ablation element comprises a pair of hollow cylindrical structures mounted in tandem with at least one lateral fenestration in the wall of each cylindrical structure in lateral alignment with each other, with one lumen within the catheter shaft in communication with the interior of one hollow cylindrical structure and a fluid connector disposed in the vicinity of the proximal end of the catheter shaft, and a second lumen within the catheter shaft in communication with the interior of the second hollow cylindrical structure and a second fluid connector disposed in the vicinity of the proximal end of the catheter shaft, at least one electrode surface within the interior of each hollow cylindrical structure connected to the electrical connector disposed in the vicinity of the proximal end of the catheter shaft by an electrical conduit, and where all external surfaces of the catheter assembly are electrically isolated from both electrode surfaces, and one electrode surface is electrically isolated from the second electrode surface, and where each electrode surface is connectable to opposite poles of a radiofrequency energy generator configured for carotid body modulation.

A vascular catheter has been conceived for carotid body modulation in a patient comprising, a catheter shaft with a caliber between approximately 3 French and 6 French, with a working length between approximately 10 cm and 25 cm, at least one ablation element mounted in the vicinity of the distal end, a mechanism configured for positioning the ablation element(s) against the wall of an external carotid artery adjacent to a carotid body, a means for providing the user with a substantially unambiguous fluoroscopic indication of the position of the ablation element(s) within the external carotid artery, and a means for connecting the ablation element to a source of ablation energy mounted in the vicinity of the proximal end, whereby the ablation element comprises a piezo-electric element configured for directed emission of ultrasonic energy, an optical mechanism configured to deflect laser energy from an axial direction to a substantially lateral direction, or a cryo-ablation element.

A vascular catheter has been conceived for carotid body modulation in a patient comprising, a catheter shaft with a caliber between approximately 3 French and 6 French, with a working length between approximately 10 cm and 25 cm, at least one ablation element mounted in the vicinity of the distal end, a mechanism configured for positioning the ablation element(s) against the wall of an external carotid artery adjacent to a carotid body, a means for providing the user with a substantially unambiguous fluoroscopic indication of the position of the ablation element(s) within the external carotid artery, and a means for connecting the ablation element to a source of ablation energy mounted in the vicinity of the proximal end, whereby the ablation element comprises at least one RF electrode mounted on the surface of an inflatable balloon, an expandable structure, an expandable cage, an expandable mesh, or an expandable braid.

A vascular catheter has been conceived for carotid body modulation in a patient comprising, a catheter shaft with a caliber between approximately 3 French and 6 French, with a working length between approximately 10 cm and 25 cm, at least one ablation element mounted in the vicinity of the distal end, a mechanism configured for positioning the ablation element(s) against the wall of an external carotid artery adjacent to a carotid body, and providing the user with a substantially unambiguous fluoroscopic indication of the position of the ablation element(s) within the external carotid artery, and a means for connecting the ablation element to a source of ablation energy mounted in the vicinity of the proximal end, whereby the mechanism comprises a push wire, and inflatable balloon, or a pull wire configured for deflecting the distal end of the catheter in a lateral direction by means of an actuator mounted in the vicinity of the proximal end of the catheter

A vascular catheter has been conceived for carotid body modulation in a patient comprising, a catheter shaft with a central lumen configured to house a deployable and retractable RF electrode from the vicinity of the distal end, a second lumen configured to house a slidable wire, an atraumatic structure mounted at the distal end of the slidable wire, an actuator configured for slidable wire positioning in the vicinity of the proximal end of the catheter, an electrode located proximal to the atraumatic structure connected to the atraumatic structure by a wire with a pre-formed bias towards lateral expansion, a slidable mechanism configured to arrest the lateral expansion bias by an actuator means located in the vicinity of the proximal end of the catheter, and an electrical connection means between the electrode and a pole of an RF generator.

A vascular catheter has been conceived for carotid body modulation in a patient comprising, a catheter shaft with a central lumen configured to house a deployable and retractable RF electrode from the vicinity of the distal end, a second RF electrode disposed on the outer surface of the catheter shaft in the vicinity of the distal end, a second lumen in the catheter shaft configured to house a slidable wire, an atraumatic structure mounted at the distal end of the slidable wire, an actuator configured for slidable wire positioning in the vicinity of the proximal end of the catheter, an electrode located proximal to the atraumatic structure connected to the atraumatic structure by a wire with a pre-formed bias towards lateral expansion, a slidable mechanism configured to arrest the lateral expansion bias by an actuator means located in the vicinity of the proximal end of the catheter, and an electrical connection means between each RF electrode and an opposing pole of an RF generator.

A vascular catheter has been conceived for carotid body modulation in a patient comprising, a catheter shaft with a caliber between approximately 3 French and 6 French, with a working length between approximately 10 cm and 25 cm, an ablation element comprising a bipolar pair of RF electrodes mounted in tandem with one of the electrodes mounted in the vicinity of the distal end configured for use within an internal carotid artery, and the second electrode being mounted proximal to the first electrode and configured for use within an external carotid artery, a mechanism configured for positioning the distal electrode against the wall of an internal carotid artery adjacent to a carotid body, and for positioning the proximal electrode against the wall of an external carotid artery adjacent to the same carotid body, a means for providing the user with a substantially unambiguous fluoroscopic indication of the position of each electrode within the carotid arteries, and a means for connecting each RF electrode to an opposite pole of an RF generator mounted in the vicinity of the proximal end, whereby said mechanism comprises a user actuate able deflectable catheter segment disposed between the distal electrode and the proximal electrode.

A vascular catheter has been conceived for carotid body modulation in a patient comprising, a catheter shaft with a caliber between approximately 3 French and 6 French, with a working length between approximately 10 cm and 25 cm having a central lumen configured to house a deployable and retractable RF electrode from the distal end, a second RF electrode disposed on the outer surface of the catheter shaft in the vicinity of the distal end, and an electrical connection means between each RF electrode and an opposing pole of an RF generator, whereby the deployable electrode is mounted at the distal end of a slidable structure comprising a pre-formed curve.

A system has been conceived for RF carotid body modulation in a patient comprising a monopolar RF ablation catheter configured for insertion into a carotid artery proximate to a carotid body, with an RF ablation electrode disposed in the vicinity of the distal end, and an indifferent RF electrode configured for use on or within a patient's body at a lateral location to a target site (e.g., carotid body, carotid body nerves, intercarotid septum), and a means to connect each electrode to an opposite pole of an RF generator.

A system has been conceived for RF carotid body modulation in a patient comprising a monopolar RF ablation catheter configured for insertion into a carotid artery proximate to a carotid body, with an RF ablation electrode disposed in the vicinity of the distal end, and an indifferent RF electrode configured for use on or within a patient's body at a lateral location to a target site (e.g., carotid body, carotid body nerves, intercarotid septum), and a means to connect each electrode to an opposite pole of an RF generator, whereby, the indifferent electrode is configured for use within an internal jugular vein, within an internal carotid artery, within a muscular structure of the neck, or on the skin of the patient's neck

A kit for carotid body modulation in a patient has been conceived comprising: an ablation catheter with an ablation element mounted in the vicinity of the distal end, a catheter shaft with a caliber between approximately 3 French and 6 French, with a working length between approximately 10 cm and 25 cm, a mechanism configured for positioning the ablation element against the wall of an external carotid artery adjacent to a carotid body, a means for providing the user with a substantially unambiguous fluoroscopic indication of the position of the ablation element within an external carotid artery, and a means for connecting the ablation element to a source of ablation energy mounted in the vicinity of the proximal end; an arterial access sheath configured for superficial temporal artery access comprising a hollow thin walled tubular structure sized to accommodate a 3 French to 6 French ablation catheter internally, with a working length between approximately 10 cm and 25 cm, a radiopaque marker in the vicinity of the distal end of the tubular structure, and a valve and a liquid port mounted in the vicinity of the proximal end; and, instructions for use comprising instructions for accessing a superficial temporal artery in a retrograde manner, and positioning the ablation catheter for carotid body modulation in a patient; wherein the ablation element is a radiofrequency electrode, bipolar radiofrequency electrodes, multiple radiofrequency electrodes, a cryo-ablation element, a virtual radiofrequency electrode, or irreversible electroporation electrodes.

A kit for carotid body modulation in a patient has been conceived comprising: an ablation catheter with a monopolar RF ablation element mounted in the vicinity of the distal end, a catheter shaft with a caliber between approximately 3 French and 6 French, with a working length between approximately 10 cm and 25 cm, a mechanism configured for positioning the monopolar RF ablation element against the wall of an external carotid artery adjacent to a carotid body, a means for providing the user with a substantially unambiguous fluoroscopic indication of the position of the monopolar RF ablation element within an external carotid artery, and a means for connecting the monopolar RF ablation element to a pole of an RF generator mounted in the vicinity of the proximal end; an arterial access sheath configured for superficial temporal artery access comprising a hollow thin walled tubular structure sized to accommodate a 3 French to 6 French monopolar RF ablation catheter internally, with a working length between approximately 10 cm and 25 cm, a radiopaque marker in the vicinity of the distal end of the tubular structure, and a valve and a liquid port mounted in the vicinity of the proximal end, an indifferent electrode configured for lateral placement to the target site (e.g., carotid body, carotid body nerves, intercarotid septum) with a connection means to the opposite pole of the RF generator, and, instructions for use comprising instructions for accessing a superficial temporal artery in a retrograde manner, and positioning the monopolar RF ablation catheter for carotid body modulation in a patient, and positioning the indifferent RF electrode in lateral position to the target site.

A kit for carotid body modulation in a patient has been conceived comprising: an ablation catheter having a catheter shaft with a central lumen configured to house a deployable and retractable RF electrode from the vicinity of the distal end, a second lumen configured to house a slidable wire, an atraumatic structure mounted at the distal end of the slidable wire, an actuator configured for slidable wire positioning in the vicinity of the proximal end of the catheter, an electrode located proximal to the atraumatic structure connected to the atraumatic structure by a wire with a pre-formed bias towards lateral expansion, a slidable mechanism configured to arrest the lateral expansion bias by an actuator means located in the vicinity of the proximal end of the catheter, and an electrical connection means between the electrode and a pole of an RF generator; an arterial access sheath configured for superficial temporal artery access comprising a hollow thin walled tubular structure sized to accommodate a 3 French to 6 French ablation catheter internally, with a working length between approximately 10 cm and 25 cm, a radiopaque marker in the vicinity of the distal end of the tubular structure, and a valve and a liquid port mounted in the vicinity of the proximal end; and, instructions for use comprising instructions for accessing a superficial temporal artery in a retrograde manner, and positioning the ablation catheter for carotid body modulation in a patient.

A kit for carotid body modulation in a patient has been conceived comprising: an ablation catheter having a catheter shaft with a central lumen configured to house a deployable and retractable RF electrode from the vicinity of the distal end, a second lumen configured to house a slidable wire, an atraumatic structure mounted at the distal end of the slidable wire, an actuator configured for slidable wire positioning in the vicinity of the proximal end of the catheter, an electrode located proximal to the atraumatic structure connected to the atraumatic structure by a wire with a pre-formed bias towards lateral expansion, a slidable mechanism configured to arrest the lateral expansion bias by an actuator means located in the vicinity of the proximal end of the catheter, and an electrical connection means between the electrode and a pole of an RF generator; an arterial access sheath configured for superficial temporal artery access comprising a hollow thin walled tubular structure sized to accommodate a 3 French to 6 French ablation catheter internally, with a working length between approximately 10 cm and 25 cm, a radiopaque marker in the vicinity of the distal end of the tubular structure, and a valve and a liquid port mounted in the vicinity of the proximal end, an indifferent electrode configured for lateral placement to the target site (e.g., carotid body, carotid body nerves, intercarotid septum) with a connection means to the opposite pole of the RF generator, and, instructions for use comprising instructions for accessing a superficial temporal artery in a retrograde manner, and positioning the ablation catheter for carotid body modulation in a patient, and positioning the indifferent electrode in a position lateral to the target site.

A kit for carotid body modulation in a patient has been conceived comprising: an ablation catheter having a catheter shaft with a caliber between approximately 3 French and 6 French, with a working length between approximately 10 cm and 25 cm having a central lumen configured to house a deployable and retractable RF electrode from the distal end, a second RF electrode disposed on the outer surface of the catheter shaft in the vicinity of the distal end, and an electrical connection means between each RF electrode and an opposing pole of an RF generator, whereby the deployable electrode is mounted at the distal end of a slidable structure comprising a pre-formed curve; an arterial access sheath configured for superficial temporal artery access comprising a hollow thin walled tubular structure sized to accommodate a 3 French to 6 French ablation catheter internally, with a working length between approximately 10 cm and 25 cm, a radiopaque marker in the vicinity of the distal end of the tubular structure, and a valve and a liquid port mounted in the vicinity of the proximal end; and, instructions for use comprising instructions for accessing a superficial temporal artery in a retrograde manner, and positioning the ablation catheter for carotid body modulation in a patient.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, an ablation element mounted in the vicinity of the distal end of the catheter shaft, a mechanism configured for positioning the ablation element against the wall of an external carotid artery in the direction of, and at the level of a target site (e.g., carotid body, carotid body nerves, intercarotid septum), and a means for connecting the ablation element to an ablation energy source; connecting the ablation element to an ablation energy source; positioning the ablation element against the wall of an external carotid artery adjacent to the target site; activating the ablation element at a level and for a duration sufficient to substantially ablate the function of the target site.

A method has been conceived for carotid body modulation in a patient comprising inserting a vascular access sheath into a superficial temporal artery, or another distal branch of an external carotid artery in a retrograde direction; inserting a monopolar RF ablation catheter though the sheath, with the ablation catheter comprising a catheter shaft, a monopolar RF ablation element mounted in the vicinity of the distal end of the catheter shaft, a mechanism configured for positioning the monopolar RF ablation element in contact with a wall of an external carotid artery in the direction of, and at the level of a target site (e.g. carotid body, carotid body nerves, intercarotid septum), and a means for connecting the electrode associated with monopolar RF ablation element to a pole of an RF generator; connecting the monopolar RF ablation element to a pole of an RF generator, and connecting an indifferent RF electrode to the second pole of the RF generator; positioning the monopolar RF ablation element against the wall of an external carotid artery adjacent to the target site; activating the RF generator to deliver RF energy at an amplitude and for a duration sufficient to substantially ablate the function of the target carotid body.

A method has been conceived for carotid body modulation in a patient comprising inserting a vascular access sheath into a superficial temporal artery, or another distal branch of an external carotid artery in a retrograde direction; inserting a monopolar RF ablation catheter though the sheath, with the ablation catheter comprising a catheter shaft, a monopolar RF ablation element mounted in the vicinity of the distal end of the catheter shaft, a mechanism configured for positioning the monopolar RF ablation element against the wall of an external carotid artery in the direction of, and at the level of a target site (e.g., carotid body, carotid body nerves, intercarotid septum), and a means for connecting the electrode associated with monopolar RF ablation element to a pole of an RF generator; then, connecting the monopolar RF ablation element to a pole of an RF generator, and connecting an indifferent RF electrode to the second pole of the RF generator; then, positioning the monopolar RF ablation element against the wall of an external carotid artery adjacent to the target site; activating the RF generator to deliver RF energy at an amplitude and for a duration sufficient to substantially ablate the function of the target carotid body, whereby the indifferent RF electrode is configured for use on or within the patient in a lateral position to the target site to direct RF energy from the monopolar RF ablation element through the target site toward the indifferent RF electrode.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, an ablation element mounted in the vicinity of the distal end of the catheter shaft, a mechanism configured for positioning the ablation element in contact with a wall of an external carotid artery in the direction of, and at the level of a target site (e.g., carotid body, carotid body nerves, intercarotid septum), and a means for connecting the ablation element to an ablation energy source; connecting the ablation element to an ablation energy source; positioning the ablation element against the wall of an external carotid artery adjacent to the target site; then, delivering ablation energy at an amplitude and for a duration sufficient to substantially ablate the function of the target carotid body, whereby the ablation element is a lateral monopolar RF electrode configured to apply RF energy to the wall of an external carotid artery and minimize applying RF energy to arterial blood, and the source of ablation energy is a radiofrequency energy generator configured for carotid body modulation.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, an ablation element mounted in the vicinity of the distal end of the catheter shaft, a mechanism configured for positioning the ablation element in contact with a wall of an external carotid artery in the direction of, and at the level of a target site (e.g., carotid body, carotid body nerves, intercarotid septum), and a means for connecting the ablation element to an ablation energy source; then, connecting the ablation element to an ablation energy source; positioning the ablation element against the wall of an external carotid artery adjacent to the target site; delivering ablation energy at an amplitude and for a duration sufficient to substantially ablate the function of the target site, whereby the ablation element is a cylindrical monopolar RF electrode with a means for substantial surface irrigation by ionic liquid, and the source of ablation energy is a radiofrequency energy generator configured for carotid body modulation.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, an ablation element mounted in the vicinity of the distal end of the catheter shaft, a mechanism configured for positioning the ablation element against the wall of an external carotid artery in the direction of, and at the level of a target site (e.g., carotid body, carotid body nerves, intercarotid septum), and a means for connecting the ablation element to an ablation energy source; connecting the ablation element to an ablation energy source; positioning the ablation element in contact with a wall of an external carotid artery adjacent to the target site; delivering ablation energy at an amplitude and for a duration sufficient to substantially ablate the function of the target carotid body, whereby the ablation element is a lateral monopolar RF electrode configured to apply RF energy to the wall of an external carotid artery and avoid applying RF energy to arterial blood, with a means for substantial surface irrigation by ionic liquid, and the source of ablation energy is a radiofrequency energy generator configured for carotid body modulation.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, an ablation element mounted in the vicinity of the distal end of the catheter shaft, a mechanism configured for positioning the ablation element in contact with a wall of an external carotid artery in the direction of, and at the level of a target site (e.g., carotid body, carotid body nerves, intercarotid septum), and a means for connecting the ablation element to an ablation energy source; connecting the ablation element to an ablation energy source; positioning the ablation element against the wall of an external carotid artery adjacent to the target site; delivering ablation energy at an amplitude and for a duration sufficient to substantially ablate the function of the target site, whereby the ablation element comprises a hollow cylindrical structure with at least one lateral fenestration, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to the electrical connector disposed in the vicinity of the proximal end of the catheter shaft by an electrical conduit, and where all external surfaces of the catheter assembly are electrically isolated from the at least one electrode surface, and the source of ablation energy is a radiofrequency energy generator configured for carotid body modulation.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, an ablation element mounted in the vicinity of the distal end of the catheter shaft, a mechanism configured for positioning the ablation element in contact with a wall of an external carotid artery in the direction of, and at the level of a target site (e.g., carotid body, carotid body nerves, intercarotid septum), and a means for connecting the ablation element to an ablation energy source; then, connecting the ablation element to an ablation energy source; positioning the ablation element in contact with the wall of an external carotid artery adjacent to the target site; delivering ablation energy at an amplitude and for a duration sufficient to substantially ablate the function of the target site, whereby the ablation element is a cylindrical bipolar pair of RF electrodes mounted in tandem, with each electrode connectable to an opposite pole of a radiofrequency energy generator configured for carotid body modulation.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, an ablation element mounted in the vicinity of the distal end of the catheter shaft, a mechanism configured for positioning the ablation element in contact with a wall of an external carotid artery in the direction of, and at the level of a target site (e.g., carotid body, carotid body nerves, intercarotid septum), and a means for connecting the ablation element to an ablation energy source; connecting the ablation element to an ablation energy source; positioning the ablation element against the wall of an external carotid artery adjacent to the target site; delivering ablation energy at an amplitude and for a duration sufficient to substantially ablate the function of the target site, whereby the ablation element is a lateral bipolar pair of RF electrodes mounted in tandem configured to apply RF energy to the wall of an external carotid artery and minimize applying RF energy to arterial blood, with each electrode connectable to an opposite pole of a radiofrequency energy generator configured for carotid body modulation.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, an ablation element mounted in the vicinity of the distal end of the catheter shaft, a mechanism configured for positioning the ablation element in contact with a wall of an external carotid artery in the direction of, and at the level of a target site (e.g., carotid body, carotid body nerves, intercarotid septum), and a means for connecting the ablation element to an ablation energy source; connecting the ablation element to an ablation energy source; positioning the ablation element against the wall of an external carotid artery adjacent to the target site; delivering ablation energy at an amplitude and for a duration sufficient to substantially ablate the function of the target site, whereby the ablation element comprises a pair of hollow cylindrical structures mounted in tandem with at least one lateral fenestration in the wall of each cylindrical structure in lateral alignment with each other, with one lumen within the catheter shaft in communication with the interior of one hollow cylindrical structure and a fluid connector disposed in the vicinity of the proximal end of the catheter shaft, and a second lumen within the catheter shaft in communication with the interior of the second hollow cylindrical structure and a second fluid connector disposed in the vicinity of the proximal end of the catheter shaft, at least one electrode surface within the interior of each hollow cylindrical structure connected to the electrical connector disposed in the vicinity of the proximal end of the catheter shaft by an electrical conduit, and where all external surfaces of the catheter assembly are electrically isolated from both electrode surfaces, and one electrode surface is electrically isolated from the second electrode surface, and where each electrode surface is connectable to opposite poles of a radiofrequency energy generator configured for carotid body modulation.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, an ablation element mounted in the vicinity of the distal end of the catheter shaft, a mechanism configured for positioning the ablation element in contact with a wall of an external carotid artery in the direction of, and at the level of a target site (e.g., carotid body, carotid body nerves, intercarotid septum), and a means for connecting the ablation element to an ablation energy source; connecting the ablation element to an ablation energy source; positioning the ablation element against the wall of an external carotid artery adjacent to the target site; delivering ablation energy at an amplitude and for a duration sufficient to substantially ablate the function of the target site, whereby the ablation element comprises a piezo-electric element configured for directed emission of ultrasonic energy, an optical mechanism configured to deflect laser energy from an axial direction to a substantially lateral direction, at least one RF electrode mounted on the surface of an inflatable balloon, at least one RF electrode mounted on the surface of an expandable structure.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, a cryo-ablation element mounted in the vicinity of the distal end of the catheter shaft, a mechanism configured for positioning the cryo-ablation element in contact with a wall of an external carotid artery in the direction of, and at the level of a target site (e.g., carotid body, carotid body nerves, intercarotid septum), and a means for connecting the cryo-ablation element to a cryogen source; connecting the cryo-ablation element to a cryogen source; positioning the cryo-ablation element against the wall of an external carotid artery adjacent to the target site; delivering ablation energy at an amplitude and for a duration sufficient to substantially ablate the function of the target site.

A method has been conceived for carotid body modulation in a patient comprising inserting a vascular access sheath into a superficial temporal artery, or another distal branch of an external carotid artery in a retrograde direction; inserting an ablation catheter though the sheath, with the ablation catheter comprising a catheter shaft, an ablation element mounted in the vicinity of the distal end of the catheter shaft, a mechanism configured for positioning the ablation element in contact with a wall of an external carotid artery in the direction of, and at the level of a target site (e.g., carotid body, carotid body nerves, intercarotid septum), and a means for connecting the ablation element to an ablation energy source; connecting the ablation element to an ablation energy source; positioning the ablation element against the wall of an external carotid artery adjacent to the target site; delivering ablation energy at an amplitude and for a duration sufficient to substantially ablate the function of the target site, whereby the mechanism comprises a push wire, an inflatable balloon, or a pull wire configured for deflecting the distal end of the catheter in a lateral direction by means of an actuator mounted in the vicinity of the proximal end of the catheter.

A method has been conceived for carotid body modulation in a patient comprising, inserting a vascular access sheath into a superficial temporal artery, or another distal branch of an external carotid artery in a retrograde direction; inserting an ablation catheter though the sheath, with the ablation catheter comprising a catheter shaft with a central lumen configured to house a deployable and retractable RF electrode from the vicinity of the distal end, a second lumen configured to house a slidable wire, an atraumatic structure mounted at the distal end of the slidable wire, an actuator configured for slidable wire positioning in the vicinity of the proximal end of the catheter, an electrode located proximal to the atraumatic structure connected to the atraumatic structure by a wire with a pre-formed bias towards lateral expansion, a slidable mechanism configured to arrest the lateral expansion bias by an actuator means located in the vicinity of the proximal end of the catheter, and an electrical connection means between the electrode and a pole of an RF generator; connecting the electrode to a pole of an RF generator; deploying the deployable electrode to a position against the wall of a carotid artery proximate to the target site (e.g., carotid body, carotid body nerves, intercarotid septum); applying RF energy to the carotid artery wall by the electrode at an amplitude and duration sufficient to substantially ablate the function of the carotid body, then optionally, determining functionality of the carotid body, and if carotid body function remains above the clinical objective; then, positioning the electrode against the wall of the internal carotid artery adjacent to the target site an applying RF energy to the wall of the internal carotid artery at an amplitude and duration sufficient to substantially further ablate carotid body function.

A method has been conceived for carotid body modulation in a patient comprising, inserting a vascular access sheath into a superficial temporal artery, or another distal branch of an external carotid artery in a retrograde direction; inserting an ablation catheter though the sheath, with the ablation catheter comprising a catheter shaft with a central lumen configured to house a deployable and retractable RF electrode from the vicinity of the distal end, a second RF electrode disposed on the outer surface of the catheter shaft in the vicinity of the distal end, a second lumen in the catheter shaft configured to house a slidable wire, an atraumatic structure mounted at the distal end of the slidable wire, an actuator configured for slidable wire positioning in the vicinity of the proximal end of the catheter, an electrode located proximal to the atraumatic structure connected to the atraumatic structure by a wire with a pre-formed bias towards lateral expansion, a slidable mechanism configured arrest the lateral expansion bias by an actuator means located in the vicinity of the proximal end of the catheter, and an electrical connection means between each RF electrode and an opposing pole of an RF generator; connecting the electrodes to an RF generator; deploying the deployable electrode to a position in contact with a wall of an internal carotid artery proximate to a target site (e.g., carotid body, carotid body nerves, intercarotid septum), and positioning the surface mounted electrode in contact with a wall of the external carotid artery adjacent to the target site; applying RF energy to the internal and external carotid artery walls by the electrodes at an amplitude and duration sufficient to substantially ablate a function of the target carotid body.

A method has been conceived for carotid body modulation in a patient comprising, inserting a vascular access sheath into a superficial temporal artery, or another distal branch of an external carotid artery in a retrograde direction; inserting an ablation catheter though the sheath, with the ablation having a catheter shaft with a caliber between approximately 3 French and 6 French, with a working length between approximately 10 cm and 25 cm, an ablation element comprising a bipolar pair of RF electrodes mounted in tandem with one of the electrodes mounted in the vicinity of the distal end configured for use within an internal carotid artery, and the second electrode being mounted proximal to the first electrode and configured for use within an external carotid artery, a mechanism configured for positioning the distal electrode against the wall of an internal carotid artery adjacent to a carotid body, and for positioning the proximal electrode against the wall of an external carotid artery adjacent to the same carotid body, a means for providing the user with a substantially unambiguous fluoroscopic indication of the position of each electrode within the carotid arteries, and a means for connecting each RF electrode to an opposite pole of an RF generator mounted in the vicinity of the proximal end, whereby said mechanism comprises a user actuate able deflectable catheter segment disposed between the distal electrode and the proximal electrode; connecting the electrodes to an RF generator; deploying the deployable electrode to a position against the wall of an internal carotid artery proximate to the target site (e.g., carotid body, carotid body nerves, intercarotid septum), and positioning the surface mounted electrode against the wall of the external carotid artery adjacent to the target site; applying RF energy to the carotid artery walls by the electrodes at an amplitude and duration sufficient to substantially ablate a function of the carotid body.

A method has been conceived for carotid body modulation in a patient comprising, inserting a vascular access sheath into a superficial temporal artery, or another distal branch of an external carotid artery in a retrograde direction; inserting an ablation catheter though the sheath, with the ablation catheter having a catheter shaft with a caliber between 3 French and 6 French, with a working length between approximately 10 cm and 25 cm having a central lumen configured to house a deployable and retractable RF electrode from the distal end, a second RF electrode disposed on the outer surface of the catheter shaft in the vicinity of the distal end, and an electrical connection means between each RF electrode and an opposing pole of an RF generator, whereby the deployable electrode is mounted at the distal end of a slidable structure comprising a pre-formed curve; connecting the electrodes to an RF generator; deploying the deployable electrode to a position against the wall of an internal carotid artery proximate to the target site (e.g., carotid body, carotid body nerves, intercarotid septum), and positioning the surface mounted electrode in contact with a wall of the external carotid artery adjacent to the target site; applying RF energy to the carotid artery walls by the electrodes at an amplitude and duration sufficient to substantially ablate a function of the carotid body.

A method has been conceived for carotid body modulation in a patient comprising inserting an ablation catheter into a superficial temporal artery, or another distal branch of an external carotid artery in the retrograde direction, with the ablation catheter comprising a catheter shaft, an ablation element mounted in the vicinity of the distal end of the catheter shaft, a mechanism configured for positioning the ablation element against the wall of an external carotid artery in the direction of, and at the level of a target site (e.g., carotid body, carotid body nerves, intercarotid septum), and a means for connecting the ablation element to an ablation energy source; connecting the ablation element to an ablation energy source; positioning the ablation element against the wall of an external carotid artery adjacent to the target site; activating the ablation element at a level and for a duration sufficient to substantially ablate the function of the target site.

The methods and systems disclosed herein may be applied to satisfy clinical needs related to treating cardiac, metabolic, and pulmonary diseases associated, at least in part, with augmented chemoreflex (e.g., high chemosensor sensitivity or high chemosensor activity) and related sympathetic activation. The treatments disclosed herein may be used to restore autonomic balance by reducing sympathetic activity, as opposed to increasing parasympathetic activity. It is understood that parasympathetic activity can increase as a result of the reduction of sympathetic activity (e.g., sympathetic withdrawal) and normalization of autonomic balance. Furthermore, the treatments may be used to reduce sympathetic activity by modulating a peripheral chemoreflex. Furthermore, the treatments may be used to reduce afferent neural stimulus, conducted via afferent carotid body nerves, from a carotid body to the central nervous system. Enhanced peripheral and central chemoreflex is implicated in several pathologies including hypertension, cardiac tachyarrhythmias, sleep apnea, dyspnea, chronic obstructive pulmonary disease (COPD), diabetes and insulin resistance, and CHF. Mechanisms by which these diseases progress may be different, but they may commonly include contribution from increased afferent neural signals from a carotid body. Central sympathetic nervous system activation is common to all these progressive and debilitating diseases. Peripheral chemoreflex may be modulated, for example, by modulating carotid body activity. The carotid body is the sensing element of the afferent limb of the peripheral chemoreflex. Carotid body activity may be modulated, for example, by ablating a carotid body or afferent nerves emerging from the carotid body. Such nerves can be found in a carotid body itself, in a carotid plexus, in an intercarotid septum, in periarterial space of a carotid bifurcation and internal and external carotid arteries, and internal jugular vein, or facial vein. Therefore, a therapeutic method has been conceived that comprises a goal of restoring or partially restoring autonomic balance by reducing or removing carotid body input into the central nervous system.

### BRIEF DESCRIPTION OF THE DRAWINGS

FIG. 1 is an illustration of a right side of a head of a patient depicting vascular access to right external carotid artery using a superficial temporal artery puncture.

FIG. 2 is a schematic illustration of a region of a carotid bifurcation comprising a carotid body.

FIGS. 3A and 3B illustrate an example of dual ablation element positioning that may effectively and safely ablate a carotid body.

FIGS. 4A and 4B illustrate an example of single ablation element positioning that may effectively and safely ablate a carotid body.

FIG. 5 is an illustration of a procedure kit for trans-temporal artery ablation of a carotid body comprising a needle, guide wire, arterial sheath and obturator, a carotid body ablation catheter, and directions-for-use.

FIG. 6 is a schematic illustration of a carotid body ablation catheter in situ utilizing access to the region of the carotid body from a superficial temporal artery puncture.

FIG. 7A is a front view illustration of the distal end of Monopolar RF Ionic Stream Carotid Body Ablation (MRF-IS-CBA) catheter. FIG. 7B is a rear view illustration of the distal end of MRF-IS-CBA catheter. FIG. 7C is a rear view illustration of the distal end of MRF-IS-CBA catheter.

FIG. 8A is a front view illustration of a distal end of Tandem Bipolar RF Ionic Stream Carotid Body Ablation (TBRF-IS-CBA) catheter. FIG. 8B is a cross sectional illustration of the distal end of TBRF-IS-CBA catheter.

FIG. 9 is a schematic illustration of TBRF-IS-CBA catheter, in situ, with access to the region of the carotid body from a superficial temporal artery puncture.

FIG. 10 is an illustration of a lateral tandem bipolar RF carotid body ablation (LTB-RF-CBA) catheter.

FIG. 11 is a schematic illustration of a carotid LTB-RF-CBA catheter in situ, with access to the region of the carotid body from a superficial temporal artery puncture.

FIGS. 12A-12G are illustrations of a Retrograde Carotid Body Ablation Bipolar (R-CBA-B) catheter.

FIG. 13 is an illustration of the distal end of a Retrograde Carotid Body Monopolar Bifurcation Coupling (R-CBA-MBC) catheter configured for use by superficial temporal artery access to the region of a carotid body.

FIG. 14A is an in situ schematic illustration of the distal end of a R-CBA-B catheter configured for use by trans-superficial temporal artery access to the region of a carotid body shown in its insertion configuration.

FIG. 14B is an in situ schematic illustration of the distal end of R-CBA-B catheter shown with the outer sheath retracted exposing ring electrode, bifurcation coupling arm, distal tip, actuator clasp, and actuator clasp wire, with the bifurcation coupling electrode docked within the central lumen of the catheter shaft.

FIG. 14C is an in situ schematic illustration of the distal end of R-CBA-B catheter shown with the bifurcation coupling electrode withdrawn from the catheter shaft central lumen for bifurcation coupling arm deployment.

FIG. 14D is an in situ schematic illustration of the distal end of R-CBA-B catheter shown with the bifurcation coupling actuator clasp and actuator clasp wire in the maximal distal position with the bifurcation coupling arm in its pre-formed biased position.

FIG. 14E is an in situ schematic illustration of the distal end of R-CBA-B catheter shown with catheter shaft advanced in the distal direction with the ring electrode positioned in opposition to bifurcation coupling electrode for bipolar ablation of carotid body.

FIG. 14F is an in situ schematic illustration of the distal end R-CBA-B catheter shown with the bifurcation coupling actuator clasp and actuator clasp wire pulled in the proximal direction to apply a pinching force to the carotid bifurcation.

FIG. 15A is an in situ schematic illustration of the distal end of a R-CBA-MBC catheter configured for use by trans-superficial temporal artery access to the region of a carotid body shown in its insertion configuration.

FIG. 15B is an in situ schematic illustration of the distal end of R-CBA-MBC catheter shown with the outer sheath retracted exposing bifurcation coupling arm, distal tip, and actuator clasp, and actuator clasp wire, with the bifurcation coupling electrode docked within the central lumen of the catheter shaft.

FIG. 15C is an in situ schematic illustration of the distal end of R-CBA-MBC catheter shown with the bifurcation coupling electrode withdrawn from the catheter shaft central lumen and pressed against the internal wall of the external carotid artery adjacent to a target site (e.g., carotid body, carotid body nerves, intercarotid septum) using a force resulting from the pre-formed lateral expansion bias of the bifurcation coupling arm.

FIG. 15D is an in situ schematic illustration of the distal end of R-CBA-MBC catheter shown with the bifurcation coupling electrode being positioned for carotid body modulation from the wall of internal carotid artery adjacent to carotid body in the instance where carotid body function remained above the determined level following ablation from the external carotid artery.

FIG. 15E is an in situ schematic illustration of the distal end of R-CBA-MBC catheter showing the bifurcation coupling actuator clasp, and actuator clasp wire pulled in the proximal direction to apply a pinching force to the carotid bifurcation.

FIG. 16A is an illustration of a Retrograde Bipolar Carotid Body Ablation Deflectable J Tip (RB-CBA-DJT) catheter configured for use through superficial temporal artery access comprising a tandem bipolar pair of RF electrodes with a user actuated segment between the electrode pair in its insertion configuration.

FIG. 16B is an illustration of R-CBA-DJT catheter in its actuated configuration.

FIG. 17A is an in situ schematic illustration RB-CBA-DJT catheter being positioned for use at the carotid bifurcation.

FIG. 17B is an in situ schematic illustration of RB-CBA-DJT catheter in position for carotid body modulation at the carotid bifurcation.

FIG. 18A is an illustration of the distal end of a Retrograde Bipolar Carotid Body Ablation Passive J Tipped (RB-CBA-PJT) catheter comprising a deployable and retractable RF ablation electrode mounted on a curved slidable structure, and a second RF ablation electrode mounted on the surface at the distal end of the RB-CBA-PJT catheter.

FIG. 18B is an illustration of RB-CBA-PJT catheter in its insertion configuration.

FIG. 18C is an illustration of RB-CBA-PJT catheter in its use configuration.

FIG. 19A is an in situ schematic illustration of a RB-CBA-PJT catheter being positioned for use at the carotid bifurcation with the ring electrode within external carotid artery at the approximate level of the target site (e.g., carotid body, carotid body nerves, intercarotid septum).

FIG. 19B is an in situ schematic illustration of RB-CBA-PJT showing the deployable and retractable electrode being deployed for use.

FIG. 19C is an in situ schematic illustration of RB-CBA-PJT showing the deployable and retractable electrode and second RF electrode in position for carotid body modulation.

FIG. 20 is a transverse schematic illustration of the carotid arteries immediately distal to the carotid bifurcation showing the relative locations of the carotid body, internal jugular vein, and sympathetic nerve.

FIG. 21 is an illustration of a Jugular Indifferent Electrode (JIE) configured for use in a major lateral vein of the neck of a patient intended to prevent RF current from damaging important non-target nervous structures medial to the carotid bifurcation saddle during RF carotid body modulation from within an external carotid body.

FIG. 22 is a schematic illustration of an RF carotid body ablation catheter in situ utilizing access to the region of the carotid body from a superficial temporal artery puncture and an JIE catheter located in the associated internal jugular vein.

FIG. 23A is a transverse sectional schematic illustration of a patient's neck depicting a monopolar RF ablation catheter residing within the external carotid artery in position for carotid body modulation, with a JIE catheter residing within the internal jugular vein, showing the RF current path between the RF catheter's ablation electrode, and the indifferent electrode on the JIE catheter.

FIG. 23B is a transverse sectional schematic illustration of a patient's neck depicting a monopolar RF ablation catheter residing within the external carotid artery in position for carotid body modulation, and a percutaneous indifferent electrode probe inserted into neck muscle adjacent to the carotid body, showing the RF current path between the RF catheter's ablation electrode, and the indifferent electrode on the indifferent electrode probe.

FIG. 23C is a transverse sectional schematic illustration of a patient's neck depicting a monopolar RF ablation catheter residing within the external carotid artery in position for carotid body modulation, and an indifferent electrode skin pan on the patient's neck adjacent to the carotid body, showing the RF current path between the RF catheter's ablation electrode, and the indifferent electrode skin pad.

### DETAILED DESCRIPTION

In the following detailed description, reference is made to the accompanying drawings, which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention.

References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

Systems, devices, and methods have been conceived for carotid body modulation (that is, to ablate fully or partially one or both carotid bodies, carotid body nerves, intercarotid septums, or peripheral chemoreceptors) to treat patients having a sympathetically mediated disease (e.g., cardiac, renal, metabolic, or pulmonary disease such as hypertension, CHF, heart failure, sleep apnea, sleep disordered breathing, diabetes, insulin resistance, atrial fibrillation, chronic kidney disease, polycystic ovarian syndrome, post MI mortality) or other disease (e.g., obesity, asthma) at least partially resulting from augmented peripheral chemoreflex (e.g., peripheral chemoreceptor hypersensitivity, peripheral chemosensor hyperactivity), heightened sympathetic activation, or an unbalanced autonomic tone. A reduction of peripheral chemoreflex or reduction of afferent nerve signaling from a carotid body (CB) resulting in a reduction of central sympathetic tone is a main therapy pathway. Higher than normal chronic or intermittent activity of afferent carotid body nerves is considered enhanced chemoreflex for the purpose of this application regardless of its cause. Other important benefits such as increase of parasympathetic tone, vagal tone and specifically baroreflex and baroreceptor activity, as well as reduction of dyspnea, hyperventilation, hypercapnea, respiratory alkalosis and breathing rate may be expected in some patients. Secondary to reduction of breathing rate additional increase of parasympathetic tone can be expected in some cases. Reduced breathing rate can lead to increased tidal lung volume, reduced dead space and increased efficiency of gas exchange. Reduced dyspnea and reduced dead space can independently lead to improved ability to exercise. Shortness of breath (dyspnea) and exercise limitations are common debilitating symptoms in CHF and COPD. Augmented peripheral chemoreflex (e.g., carotid body activation) leads to increases in sympathetic nervous system activity, which is in turn primarily responsible for the progression of chronic disease as well as debilitating symptoms and adverse events seen in our intended patient populations. Carotid bodies contain cells that are sensitive to partial pressure of oxygen and carbon dioxide in blood plasma. Carotid bodies also may respond to blood flow, pH acidity, glucose level in blood and possibly other variables. Thus carotid body modulation may be a treatment for patients, for example having hypertension, heart disease or diabetes, even if chemosensitive cells are not activated.

An inventive treatment, endovascular carotid body modulation via a trans-superficial-temporal-artery approach, may involve gaining endovascular access to a patient's superficial temporal artery, inserting a catheter in the patient's vascular system, positioning a distal region of the catheter in a vessel proximate a carotid body (e.g., in a common carotid artery, internal carotid artery, external carotid artery, at a carotid bifurcation, proximate an intercarotid septum), positioning an ablation element (e.g., RF electrode) proximate to a target site (e.g., a carotid body, an afferent nerve associated with a carotid body, a peripheral chemosensor, an intercarotid septum), and delivering an ablation agent (e.g., RF energy) from the ablation element to ablate the target site. Several methods and devices for carotid body modulation are described.

### Targets:

To inhibit or suppress a peripheral chemoreflex, anatomical targets for ablation (also referred to as targeted tissue, target ablation sites, or target sites) may include at least a portion of at least one carotid body, an aortic body, nerves associated with a peripheral chemoreceptor (e.g., carotid body nerves, carotid sinus nerve, carotid plexus), small blood vessels feeding a peripheral chemoreceptor, carotid body parenchyma, chemosensitive cells (e.g., glomus cells), tissue in a location where a carotid body is suspected to reside (e.g., a location based on pre-operative imaging or anatomical likelihood), an intercarotid septum, a portion of an intercarotid septum or a combination thereof. As used herein, ablation of a carotid body or carotid body modulation may refer to ablation of any of these target ablation sites.

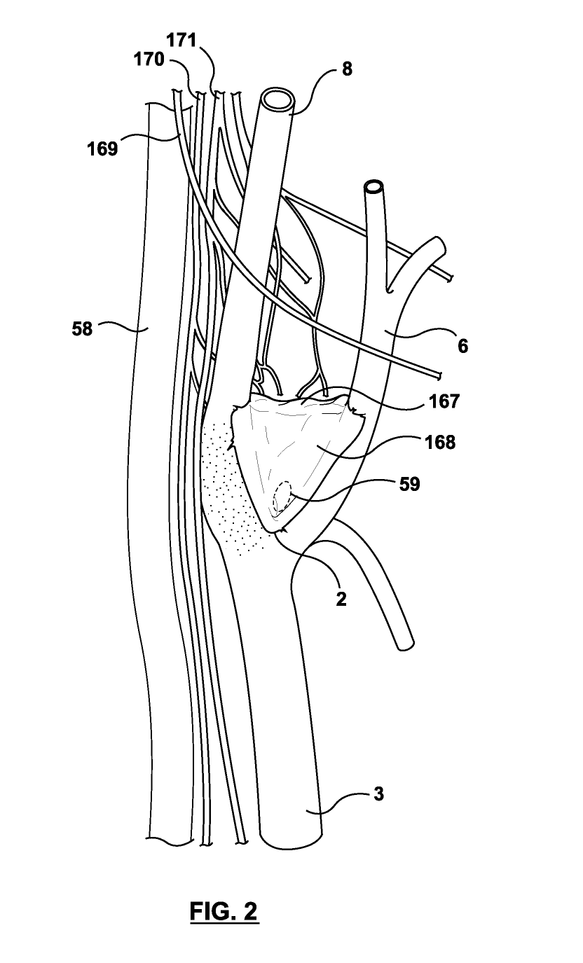

Shown in FIG. 2, a carotid body (CB) 59, housing peripheral chemoreceptors, modulates sympathetic tone through direct signaling to the central nervous system. Carotid bodies represent a paired organ system located near a bifurcation 2 of a common carotid artery 3 bilaterally, that is, on both sides of the neck. The common carotid artery 3 bifurcates to an internal carotid artery 8 and an external carotid artery 6. Typically, in humans each carotid body is approximately the size of a 2.5-5 mm ovoid grain of rice and is innervated both by the carotid sinus nerve (CSN, a branch of the glossopharyngeal nerve), and the ganglioglomerular (sympathetic) nerve of the nearby superior cervical ganglion. Infrequently other shapes are encountered. The CB is the most perfused organ per gram weight in the body and receives blood via an arterial branch or branches typically arising from internal or external carotid artery.

An intercarotid septum 168 (also referred to as carotid septum) shown in FIGS. 2, 3A, 3B, 4A, and 4B is herein defined as a wedge or triangular segment of tissue with the following boundaries: A saddle of a carotid bifurcation 2 defines a caudal aspect (an apex) of a carotid septum 168; Facing walls of internal 8 and external 6 carotid arteries define two sides of a carotid septum; A cranial boundary 167 of a carotid septum extends between these arteries and may be defined as cranial to a carotid body but caudal to any important non-target nerve structures (e.g., hypoglossal nerve) that might be in the region, for example a cranial boundary may be about 7 mm to 15 mm (e.g., about 10 mm) from the saddle of the carotid bifurcation; Medial 169 and lateral 170 walls of the carotid septum 168 are generally defined by planes approximately tangent to the internal and external carotid arteries; One of the planes is tangent to the lateral wall of the internal and external carotid arteries and the other plane is tangent to the medial walls of these arteries. An intercarotid septum is between the medial and lateral walls. The medial plane of an intercarotid septum may alternatively be defined as a carotid sheath on a medial side of a septum or within about 2 mm outside of the medial side of the carotid sheath. An intercarotid septum 168 may contain a carotid body 59 and is typically absent of important non-target nerve structures such as a vagus nerve, important non-target sympathetic nerves, or a hypoglossal nerve. Therefore, creating an ablation that is maintained within an intercarotid septum may effectively modulate a carotid body while safely avoiding collateral damage of important non-target nerve structures. An intercarotid septum may include some baroreceptors or baroreceptor nerves. An intercarotid septum may also include small blood vessels, nerves associated with the carotid body, and fat.

Carotid body nerves are anatomically defined herein as carotid plexus nerves and carotid sinus nerves, which converge into carotid body nerves approximately above the bifurcation. Carotid body nerves are functionally defined herein as nerves that conduct information from a carotid body to a central nervous system.

An ablation may be focused exclusively on targeted tissue, or be focused on the targeted tissue while safely ablating tissue proximate to the targeted tissue (e.g., to ensure the targeted tissue is ablated or as an approach to gain access to the targeted tissue). An ablation may be as big as a peripheral chemoreceptor (e.g., carotid body or aortic body) itself, somewhat smaller, or bigger and can include tissue surrounding the chemoreceptor such as blood vessels, adventitia, fascia, small blood vessels perfusing the chemoreceptor, or nerves connected to and innervating the glomus cells. An intercarotid plexus or carotid sinus nerve maybe a target of ablation with an understanding that some baroreceptor nerves will be ablated together with carotid body nerves. Baroreceptors are distributed in the human arteries and have high degree of redundancy.

Tissue may be ablated to inhibit or suppress a chemoreflex of only one of a patient's two carotid bodies. Alternatively, a carotid body modulation procedure may involve ablating tissue to inhibit or suppress a chemoreflex of both of a patient's carotid bodies. For example a therapeutic method may include ablation of one carotid body, measurement of resulting chemosensitivity, sympathetic activity, respiration or other parameter related to carotid body hyperactivity and ablation of the second carotid body if needed to further reduce chemosensitivity following unilateral ablation. The decision to ablate one or both carotid bodies may be based on pre-procedure testing or on patient's anatomy.

An embodiment of a therapy may substantially reduce chemoreflex without excessively reducing the baroreflex of the patient. The proposed ablation procedure may be targeted to substantially spare the carotid sinus, baroreceptors distributed in the walls of carotid arteries (e.g., internal carotid artery), and at least some of the carotid sinus baroreceptor nerves that conduct signals from said baroreceptors. For example, the baroreflex may be substantially spared by targeting a limited volume of ablated tissue possibly enclosing the carotid body, tissues containing a substantial number of carotid body nerves, tissues located in periadventitial space of a medial segment of a carotid bifurcation, or tissue located at the attachment of a carotid body to an artery. Said targeted ablation is enabled by visualization of the area or carotid body itself, for example by CT, CT angiography, MRI, ultrasound sonography, IVUS, OCT, intracardiac echocardiography (ICE), trans-esophageal echocardiography (TEE), fluoroscopy, blood flow visualization, or injection of contrast, and positioning of an instrument in the carotid body or in close proximity while avoiding excessive damage (e.g., perforation, stenosis, thrombosis) to carotid arteries, baroreceptors, carotid sinus nerves or other important non-target nerves such as vagus nerve or sympathetic nerves located primarily outside of the carotid septum. CT angiography and ultrasound sonography have been demonstrated to locate carotid bodies in most patients. Thus imaging a carotid body before ablation may be instrumental in (a) selecting candidates if a carotid body is present, large enough and identified and (b) guiding therapy by providing a landmark map for an operator to guide an ablation instrument to the carotid septum, center of the carotid septum, carotid body nerves, the area of a blood vessel proximate to a carotid body, or to an area where carotid body itself or carotid body nerves may be anticipated. It may also help exclude patients in whom the carotid body is located substantially outside of the carotid septum in a position close to a vagus nerve, hypoglossal nerve, jugular vein or some other structure that can be endangered by ablation. In one embodiment, only patients with a carotid body substantially located within the intercarotid septum may be selected for ablation therapy. Pre-procedure imaging can also be instrumental in choosing the right catheter depending on a patient's anatomy. For example a catheter with more space between arms can be chosen for a patient with a wider septum.

Once a carotid body is ablated, surgically removed, or denervated, the carotid body function (e.g., carotid body chemoreflex) does not substantially return in humans (in humans aortic chemoreceptors are considered undeveloped). To the contrary, once a carotid sinus baroreflex is removed (such as by resection of a carotid sinus nerve) it is generally compensated, after weeks or months, by the aortic or other arterial baroreceptor baroreflex. Thus, if both the carotid chemoreflex and baroreflex are removed or substantially reduced, for example by interruption of the carotid sinus nerve or intercarotid plexus nerves, baroreflex may eventually be restored while the chemoreflex may not. The consequences of temporary removal or reduction of the baroreflex can be in some cases relatively severe and require hospitalization and management with drugs, but they generally are not life threatening, terminal or permanent. Thus, it is understood that while selective removal of carotid body chemoreflex with baroreflex preservation may be desired, it may not be absolutely necessary in some cases.

### Ablation:

The term “ablation” may refer to the act of altering tissue to suppress or inhibit its biological function or ability to respond to stimulation permanently or for an extended period of time (e.g., greater than 3 weeks, greater than 6 months, greater than a year, for several years, or for the remainder of the patient's life). For example, ablation may involve, but is not limited to, thermal necrosis (e.g., using energy such as thermal energy, radiofrequency electrical current, direct current, microwave, ultrasound, high intensity focused ultrasound, and laser), cryogenic ablation, irreversible electroporation, selective denervation, embolization (e.g., occlusion of blood vessels feeding the carotid body), artificial sclerosing of blood vessels, mechanical impingement or crushing, surgical removal, chemical ablation, or application of radiation causing controlled necrosis (e.g., brachytherapy).

Carotid Body Modulation (CBM) and Carotid Body Ablation (CBA) may be used interchangeably and herein refers to ablation of a target tissue wherein the desired effect is to reduce or remove the afferent neural signaling from a chemosensor (e.g., carotid body) or reducing a chemoreflex. Chemoreflex or afferent nerve activity cannot be directly measured in a practical way, thus indexes of chemoreflex such as chemosensitivity can sometimes be used instead. Chemoreflex reduction is generally indicated by a reduction of an increase of ventilation and respiratory effort per unit of blood gas concentration, saturation or blood gas partial pressure change or by a reduction of central sympathetic nerve activity in response to stimulus (such as intermittent hypoxia or infusion of a drug) that can be measured directly. Sympathetic nerve activity can be assessed indirectly by measuring activity of peripheral nerves leading to muscles (MSNA), heart rate (HR), heart rate variability (HRV), production of hormones such as renin, epinephrine and angiotensin, and peripheral vascular resistance. All these parameters are measurable and their change can lead directly to the health improvements. In the case of CHF patients blood pH, blood PCO₂, degree of hyperventilation and metabolic exercise test parameters such as peak VO₂, and VE/VCO₂ slope are also important. It is believed that patients with heightened chemoreflex have low VO₂ and high VE/VCO₂ slope measured during cardiopulmonary stress test (indexes of respiratory efficiency) as a result of, for example, tachypnea and low blood CO₂. These parameters are also related to exercise limitations that further speed up patient's status deterioration towards morbidity and death. It is understood that all these indexes are indirect and imperfect and intended to direct therapy to patients that are most likely to benefit or to acquire an indication of technical success of ablation rather than to prove an exact measurement of effect or guarantee a success. It has been observed that some tachyarrhythmias in cardiac patients are sympathetically mediated. Thus, carotid body modulation may be instrumental in treating reversible atrial fibrillation and ventricular tachycardia.

In the context of this disclosure ablation includes denervation, which means destruction of nerves or their functional destruction, meaning termination of their ability to conduct signals. Selective denervation may involve, for example, interruption of afferent nerves from a carotid body while substantially preserving nerves from a carotid sinus, which conduct baroreceptor signals. Another example of selective denervation may involve interruption of nerve endings terminating in chemo sensitive cells of carotid body, a carotid sinus nerve, or intercarotid plexus which is in communication with both a carotid body and some baroreceptors wherein chemoreflex or afferent nerve stimulation from the carotid body is reduced permanently or for an extended period of time (e.g., years) and baroreflex is substantially restored in a short period of time (e.g., days or weeks). As used herein, the term “ablate” refers to interventions that suppress or inhibit natural chemoreceptor or afferent nerve functioning, which is in contrast to electrically neuromodulating or reversibly deactivating and reactivating chemoreceptor functioning (e.g., with an implantable electrical stimulator/blocker).

Carotid body modulation may include methods and systems for the thermal ablation of tissue via thermal heating or cooling (freezing) mechanisms. Thermal ablation may be achieved due to a direct effect on tissues and structures that are induced by the thermal stress. Additionally or alternatively, the thermal disruption may at least in part be due to alteration of vascular or peri-vascular structures (e.g., arteries, arterioles, capillaries or veins), which perfuse the carotid body and neural fibers surrounding and innervating the carotid body (e.g., nerves that transmit afferent information from carotid body chemoreceptors to the brain). Additionally or alternatively thermal disruption may be due to a healing process, fibrosis, or scarring of tissue following thermal injury, particularly when prevention of regrowth and regeneration of active tissue is desired. As used herein, thermal mechanisms for ablation may include both thermal necrosis or thermal injury or damage (e.g., via sustained heating, convective heating or resistive heating or combination). Thermal heating mechanisms may include raising the temperature of target neural fibers above a desired threshold, for example, above a body temperature of about 37° C. e.g., to achieve thermal injury or damage, or above a temperature of about 45° C. (e.g., above about 60° C.) to achieve thermal necrosis. It is understood that both time of heating, rate of heating and sustained hot or cold temperature are factors in the resulting degree of injury. Thermal-cooling mechanisms for ablation may include reducing the temperature of target neural fibers below a desired threshold (e.g., to achieve freezing thermal injury). It is generally accepted that temperatures below −40° C. applied over a minute or two results in irreversible necrosis of tissue and scar formation. It is recognized that tissue ablation by cold involves mechanisms of necrosis and apoptosis. At a low cooling rate freeze, tissue is destroyed by cellular dehydration and at high cooling rate freeze by intracellular ice formation and lethal rupture of plasma membrane.

In addition to raising or lowering temperature during thermal ablation, a length of exposure to thermal stimuli may be specified to affect an extent or degree of efficacy of the thermal ablation. For example, the length of exposure to thermal stimuli may be for example, longer than or equal to about 30 seconds, or even longer than or equal to about 2 minutes. Furthermore, the length of exposure can be less than or equal to about 10 minutes, though this should not be construed as the upper limit of the exposure period. A temperature threshold, or thermal dosage, may be determined as a function of the duration of exposure to thermal stimuli. Additionally or alternatively, the length of exposure may be determined as a function of the desired temperature threshold. These and other parameters may be specified or calculated to achieve and control desired thermal ablation.

In some embodiments, ablation of carotid body or carotid body nerves may be achieved via direct application of ablative energy to target tissue. For example, an ablation element may be applied at least proximate to the target, or an ablation element may be placed in a vicinity of a chemosensor (e.g., carotid body). In other embodiments, thermally-induced ablation may be achieved via indirect generation or application of thermal energy to the target neural fibers, such as through application of an electric field (e.g., radiofrequency, alternating current, and direct current), high-intensity focused ultrasound (HIFU), ultrasound, laser irradiation, or microwave radiation, to the target neural fibers. For example, thermally induced ablation may be achieved via delivery of a pulsed or continuous thermal electric field to the target tissue such as RF and pulsed RF, the electric field being of sufficient magnitude or duration to thermally induce ablation of the target tissue (e.g., to heat or thermally ablate or cause necrosis of the targeted tissue). Additional and alternative methods and apparatuses may be utilized to achieve ablation, as described hereinafter.

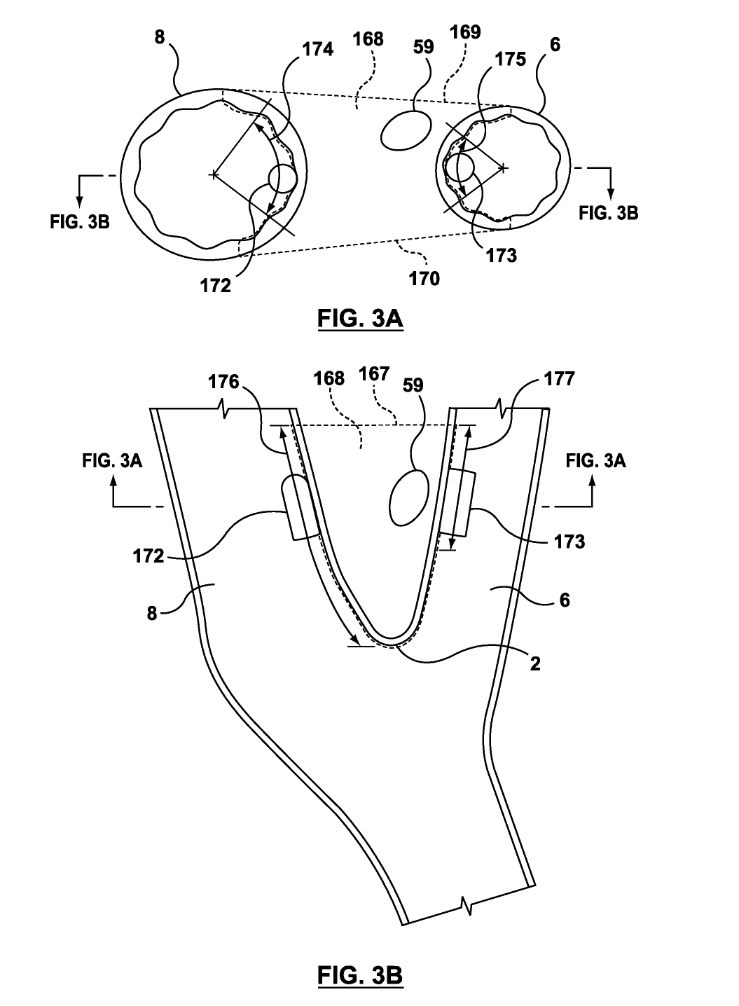

FIGS. 3A and 3B illustrate an example of dual ablation element positioning that may effectively and safely ablate a carotid body 59. FIG. 3A shows, outlined with a dashed line, a transverse cross-section of an intercarotid septum 168 bordered by an internal carotid artery 8 and an external carotid artery 6. In this embodiment, a first ablation element 172 is placed in the internal carotid artery 8 in contact with the vessel wall within a vessel wall arc 174 directed toward the external carotid artery; a second ablation element 176 is placed in the external carotid artery 6 in contact with the vessel wall within a vessel wall arc 175 directed toward the internal carotid artery. Each vessel wall arc 174 and 175 is contained within limits of the intercarotid septum 168 and comprises an arc length no greater than about 25% (e.g., about 15 to 25%) of the circumference of the respective vessel. In this example, the ablation elements 172 and 173 may be bipolar radiofrequency electrodes or irreversible electroporation electrodes wherein electrical current is passed from one electrode to the other through the intercarotid septum. Placement of ablation elements as described may facilitate the creation of an ablation that is contained within the intercarotid septum, thus avoiding important non-target nerves that reside outside the septum, and an ablation that is significantly large (e.g., extending approximately from the internal carotid artery to the external carotid artery) to effectively modulate a carotid body. FIG. 3B shows, outlined with a dashed line, a longitudinal cross-section of an intercarotid septum 168 bordered by an internal carotid artery 8, an external carotid artery 6, a saddle of a carotid bifurcation 2 and a cranial boundary 167 that is between about 7 to 15 mm cranial from the saddle 2 (e.g., about 10 mm from the saddle). The first ablation element 172 is placed in the internal carotid artery 8 in contact with the vessel wall within a first range 176; a second ablation element 173 is placed in the external carotid artery 6 in contact with the vessel wall within a second range 177. The first range 176 may extend from the inferior apex of the bifurcation saddle 2 to the cranial boundary 167 of the septum (e.g., about 7 to 15 mm from the apex, or about 10 mm from the apex). The second range 177 may extend from a position about 4 mm superior from the bifurcation saddle 2 to the cranial boundary 167 of the septum (e.g., between about 4 mm and 7 to 15 mm from the bifurcation saddle). The electrodes 172 and 173 may be equidistant from the saddle 2 or they may be unequal distances from the saddle.

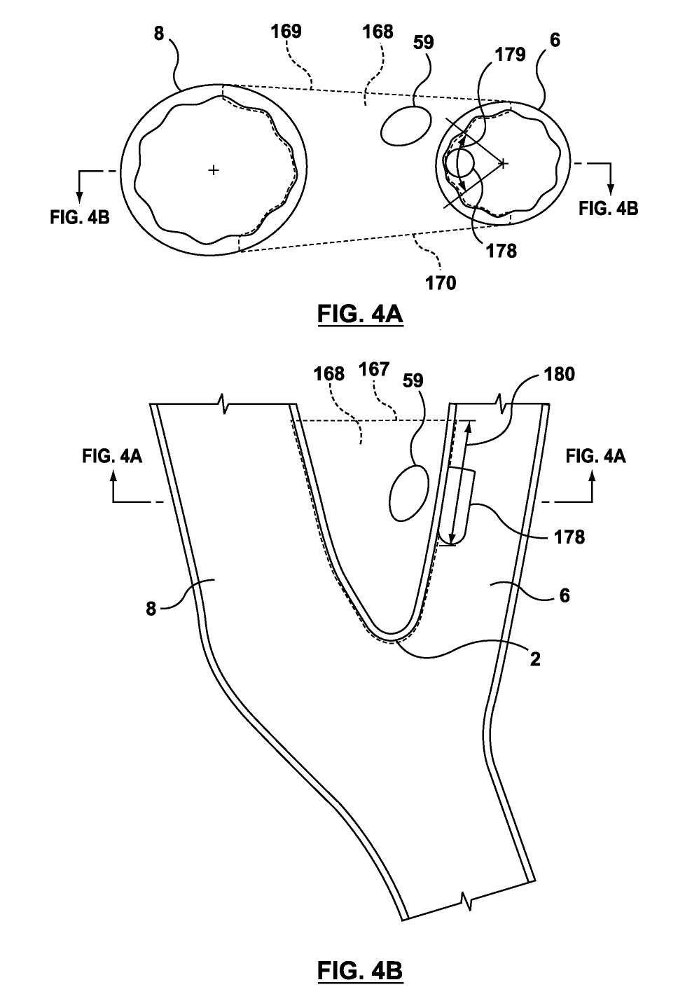

FIGS. 4A and 4B illustrate an example of single ablation element positioning that may effectively and safely ablate a carotid body 59. FIG. 4A shows, outlined with a dashed line, a transverse cross-section of an intercarotid septum 168 bordered by an internal carotid artery 8 and an external carotid artery 6. In this embodiment, a single ablation element 178 is placed in the eternal carotid artery 6 in contact with the vessel wall within a vessel wall arc 179. Vessel wall arc 179 is contained within limits of the intercarotid septum 168 and comprises an arc length no greater than about 25% (e.g., about 15 to 25%) of the circumference of the vessel and is directed toward the internal carotid artery 8. In this example, single ablation element 178 may be a monopolar radiofrequency electrode wherein electrical current is passed from one electrode to an indifferent electrode, not shown. An indifferent electrode, also known as a dispersive electrode, may be placed upon the patient's body, or within the body, for example in a vessel such as an internal carotid artery or jugular vein. Placement of ablation element 178 as described may facilitate the creation of an ablation that is contained within the intercarotid septum, thus avoiding important non-target nerves that reside outside the septum, and an ablation that is significantly large (e.g., extending approximately from the internal carotid artery to the external carotid artery) to effectively modulate a carotid body. FIG. 4B shows, outlined with a dashed line, a longitudinal cross-section of an intercarotid septum 168 bordered by an internal carotid artery 8, an external carotid artery 6, a saddle of a carotid bifurcation 2 and a cranial boundary 167 that is between about 7 to 15 mm cranial from the saddle 2. Ablation element 178 is placed in the external carotid artery 6 in contact with the vessel wall within a second range 180. Range 180 may extend from a position about 4 mm superior from the bifurcation saddle 2 to the cranial boundary 167 of the septum (e.g., between about 4 mm and 7 to 15 mm from the bifurcation saddle).

### Trans-Superficial Temporal Artery Carotid Body Modulation

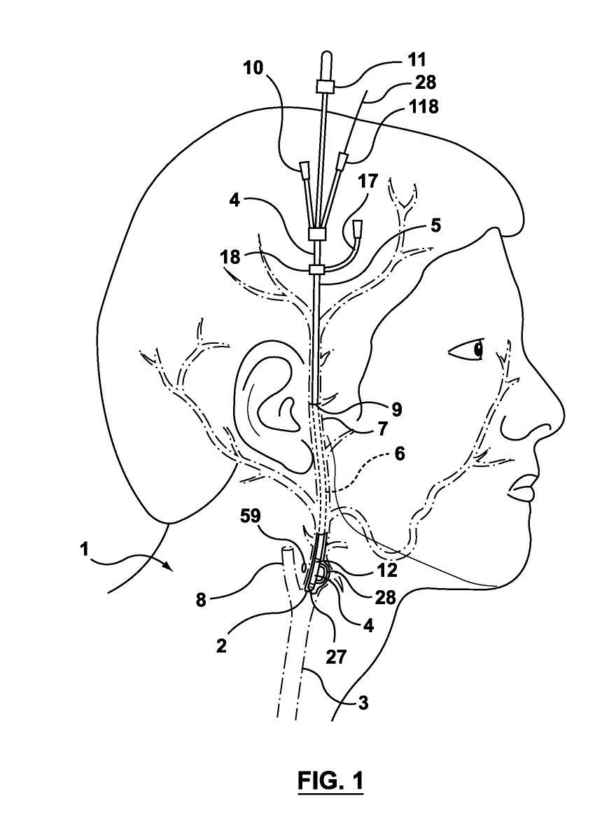

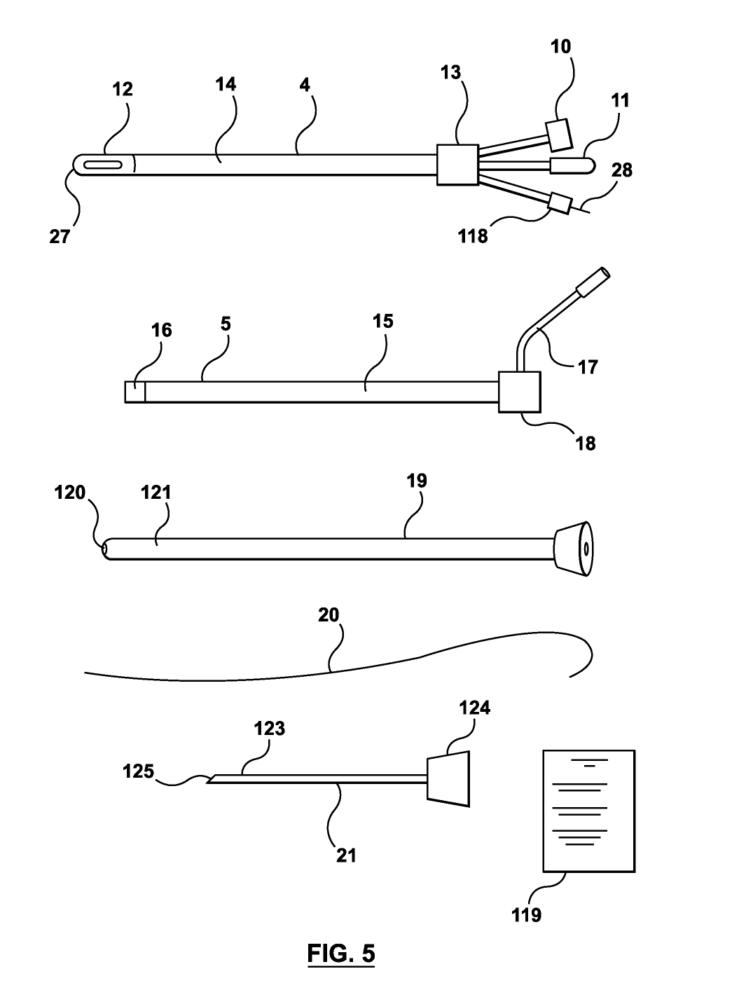

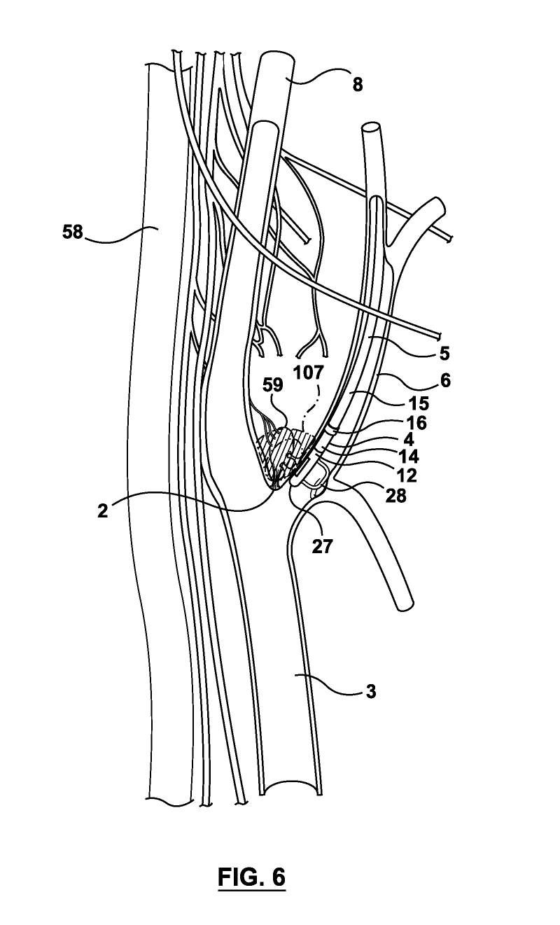

FIG. 1 is a schematic illustration of a right side of a head of a patient 1 depicting vascular access to a right external carotid artery 6 using the right superficial temporal artery 7 for the purpose of carotid body modulation. As depicted, a carotid body modulation catheter 4 is shown in position for ablation of the right carotid body 59 located in the vicinity of the carotid bifurcation 2 between the internal carotid artery 8 and the external carotid artery 6, which are the two major branches of common carotid artery 3. Ablation element 12 is shown being pushed against a wall of external carotid artery 6 in the direction of carotid body 59 by push wire 28. Ablation catheter 4 is placed into external carotid artery 6 through introducer sheath assembly 5. Introducer sheath assembly 5 is inserted into the superficial temporal artery 7, through superficial temporal puncture 9 using a superficial temporal artery access kit depicted in FIG. 5 and described in detail below. Alternatively, the superficial temporal artery may be accessed using a surgical cut-down, which may, or may not utilize introducer sheath assembly 5. Carotid body modulation catheter 4 comprises a fluid channel between the vicinity of distal tip 27, and fluid connector 10, for injection of fluids, including contrast agents into the vicinity of carotid bifurcation 2 to facilitate radiological, or ultrasonic guidance for positioning ablation element 12 against the wall of external carotid artery 6 as shown. In addition to the use of contrast agents, ablation element 12 and/or push wire 28 may be configured to provide for an unambiguous identification of position within the vasculature under radiographic or ultrasonic imaging. Ablation catheter 4 may be configured to translate rotational forces from the proximal end of the catheter residing outside of patient 1 body to the distal tip 27 of ablation catheter 4 to facilitate radial positioning ablation element 12, which may comprise a knitted, coiled or woven structure within the shaft of ablation catheter 4. Ablation element 12 is connectable to an ablation energy source, not shown, using ablation energy connector 11. Also depicted is push wire port 118, which is configured to fixture push wire 28 in its desired position. Superficial temporal arteries in adults typically range from approximately 2.25 mm in diameter to 3.25 mm in diameter, therefore, to ensure continued blood flow past superficial temporal artery puncture 9, and to avoid distal thrombosis, the caliber of introducer sheath assembly 5 or ablation catheter 4 should be smaller than superficial temporal artery 7 (e.g., less than 3.25 mm, about 2 mm, between about 1 and 2 mm, between 3 and 6 FR). As depicted, carotid body modulation catheter 4 is a generic representation of a range of carotid body modulation catheter types. Ablation element 12 may be configured for mono-polar or bipolar radiofrequency energy ablation, cryo-ablation, laser ablation, ultrasound ablation, or another ablation modality. As depicted, push wire 28 is used to push ablation element 28 against the wall of external carotid artery 6, however, there are alternative mechanisms that could be used, including using an internal pull wire to laterally deflect ablation element 12 against the wall of external carotid artery 6, or another mechanism may be used such as an expandable balloon, cage, or mesh.

FIG. 5 is an illustration of a procedure kit for trans-temporal artery ablation of a carotid body comprising a needle 21, guidewire 20, arterial introducer sheath 5, obturator 19, carotid body modulation catheter 4, and instructions for use 119. As depicted carotid body modulation catheter 4 comprises a catheter shaft with a caliber between approximately 3 French and 6 French, and a working length between approximately 10 cm and 25 cm. The working length is the distance between distal tip 27 and proximal terminal 13. Disposed in the vicinity of distal tip 27 is ablation element 12. As depicted, ablation element 12 is a lateral ablation element, where ablation is applied to the wall of an external carotid artery during carotid body ablation while avoiding ablation energy from being applied to arterial blood surrounding ablation element 12. Alternatively, an ablation element could be configured to apply ablation energy circumferentially, and to the wall of an internal carotid artery 8 in addition to the wall of an external carotid artery 6. Also, an ablation element may be configured to apply one of multiple ablation energies including radiofrequency (RF) energy, microwave energy, ultrasonic energy, irreversible electroporation, or cryo ablation energy. Catheter shaft 14 may be constructed of a flexible polymer, and may comprise a woven structure within its wall configured to translate user induced rotational force from its proximal end to its distal end with high fidelity to position ablation element 12 in rotational position for carotid body modulation. Carotid body modulation catheter 4 is depicted with a push wire 28 mechanism which is used to deploy and press ablation element 12 against the wall of an external carotid artery to facilitate carotid body modulation. However, alternative mechanisms may be used including using an internal pull wire configured for deflecting the distal end of the catheter shaft in a lateral direction, using an inflatable balloon to press an ablation element against the wall of an external carotid artery 6, or a bifurcation coupling mechanism that engages an internal carotid artery 8, and an external carotid artery 6, for example using a pinching force. Fluid connector 10 is in fluidic communication with a fluid port in the vicinity of ablation element 12, and may be used to inject an imaging contrast agent to aid in positioning ablation element 12 for carotid body modulation, and may be used to irrigate the vicinity of ablation element 12 with an ionic fluid to cool ablation element 12 or to displace arterial blood from the vicinity of ablation element 12. Ablation energy connector 11 is in communication with ablation element 12 and is used to connect ablation element 12 to a source of ablation energy. Introducer sheath 5 comprises sheath tube 15 comprising a thin walled hollow structure, introducer valve 18, fluid connector 17, and radiopaque marker 16. Sheath tube 15 has an inner diameter sized to house a catheter that is less than about 6 French (e.g., between about 3 to 6 French) and has a working length of approximately 10 cm to 25 cm, with the working length being the distance from the distal end, to introducer valve 18. Sheath tube 15 is configured for a specific caliber carotid body modulation catheter where the inner diameter of sheath tube 15 is a fraction of a millimeter larger than the outside diameter of the corresponding ablation catheter. Sheath tube 15 has a wall thickness between approximately 0.25 mm and 0.75 mm, and is an extrusion of a flexible polymeric material, which may be a polyurethane, polyethylene or other polymeric compound typically used in vascular catheter and sheath construction. Radiopaque marker 16 is bonded to sheath tube 15 in the vicinity of its distal tip and comprises a thin walled ring of radiopaque metal, or a paint comprising a radiopaque metal. Introducer valve 18 comprises an elastomeric valve configured to prevent blood from exiting the sheath when inserted into a superficial temporal artery, with, or without the ablation catheter 4 inserted into introducer sheath 5. Those skilled in the art of introducer sheath construction are familiar with introducer valve design and construction, therefore no further description is warranted. Fluid connector 17 is in fluidic communication with the inner lumen of introducer tube 15, and is used to insert and remove fluid from the introducer tube. Obturator 19 is configured to facilitate insertion of introducer sheath 5 into a superficial temporal artery. Obturator 19 comprises obturator shaft 121, central guidewire lumen 120, and guidewire valve 122. Obturator shaft 121 is configured with an outer diameter approximately the same as the corresponding carotid body modulation catheter 4, and has a working length approximately 0.5 cm to 2 cm longer than the working length of the corresponding introducer sheath 5. Obturator shaft 121 has a bullet shape formed on the distal end, and guidewire valve 122 mounted in the vicinity of the proximal end. Guidewire lumen a 120 is sized to accommodate a guidewire between approximately 0.014″ to 0.038″ and traverses the entire length of obturator shaft 121. Guidewire valve 122 is sized to accommodate the same size guidewire as guidewire lumen 120. Guidewire valve 122 is configured to prevent blood from exiting through guidewire lumen 120 during introducer sheath 5 insertion into a superficial temporal artery. Those skilled in the art of obturator construction are familiar with guidewire valve design and construction, therefore, no further description is warranted. Guidewire 20 is between approximately 0.014″ and 0.038″ and corresponds to the size of guidewire lumen 120 on obturator 19. Guidewire 20 has a length of approximately 20 cm to 50 cm, and may be uniform stiffness, or may have a distal end that is relatively floppy. Those skilled in the art of guidewire construction are familiar with guidewire design and construction, therefore, no further description is warranted. Puncture needle 21 comprises a hypotube shaft 123, and needle hub 124. hypotube shaft 123 has an inner diameter that is slightly larger than corresponding guidewire 20, which allows guidewire 20 to slide freely within hypotube shaft 123. Needle shaft 123 has a sharpened distal tip 125 configured for puncture of the skin and insertion into a superficial temporal artery. Needle hub 124 is a female luer fitting configured for attachment of a syringe or Tuohy-Borst connector. Those skilled in the art of puncture needle construction are familiar with puncture needle design and construction, therefore, no further description is warranted. Directions-for-use 119 may comprise directions for: palpating a superficial temporal artery, puncturing the skin and inserting puncture needle 21 into the superficial temporal artery, inserting guidewire 20 through needle 21; removing needle 21 from the superficial temporal artery while leaving guidewire 20 in place; inserting obturator 19 into introducer sheath 5; sliding introducer sheath 5 and obturator into the superficial temporal artery over guidewire 20; removing obturator 19 while leaving introducer sheath 5 in place; inserting carotid body modulation catheter 4 into the superficial temporal artery through introducer sheath 5; positioning ablation element 12 adjacent to a carotid body and pressing ablation element 12 against the wall of an external carotid artery using a pressing mechanism; connecting carotid body modulation catheter 4 to an ablation energy source; selecting ablation energy parameters; activating and deactivating; assessing ablation effectiveness; and further directions based on ablation effectiveness. Directions-for-use 119 may further indicate that an indication for use is carotid body modulation or ablation via a trans-temporal artery approach and may describe patients who are indicated for carotid body modulation via superficial temporal artery puncture, patients who are contra-indicated for carotid body modulation via superficial temporal artery puncture, complications which be expected, and warning of potential adverse events.

FIG. 6 is a schematic illustration of a carotid body modulation catheter 4 in situ utilizing access to the region of carotid body 59 from a superficial temporal artery puncture. As depicted, carotid body modulation catheter 4 is in ablation position immediately following an ablation as indicated by ablation zone 107. Ablation element 12 is being pushed against the wall of external carotid artery 6 immediately distal to carotid bifurcation 2 and immediately adjacent to carotid body 59. Carotid body modulation catheter 4 is a generic representation of carotid body modulation catheter configured for ablation of carotid body 59 by means of superficial temporal artery puncture. Ablation element 12 may be configured for ablation of carotid body 59 using an ablation agent that may comprise electrical joule effect energy, electromagnetic radiation, acoustic energy, cryogenic ablation, or chemo-ablation. Ablation element 12 may be configured for lateral ablation where application of the ablation agent is directed towards carotid body 59, or where the ablation energy is applied circumferentially. Ablation element 12 may comprise a mechanism that punctures the wall of external carotid artery 6 and delivers an ablation agent into the periadventitial tissue in the vicinity of carotid body 59. Ablation element 12 may comprise markers to provide the user with an indication of the location and orientation of ablation element 12 within the carotid vasculature. Markers may include radiopaque markers, ultrasonic imaging markers, or electromagnetic navigational beacons. Ablation element 12 is connected to a source of an ablation agent by means of a conduit within catheter shaft 14. Push wire 28 is a generic representation of a mechanism configured to press ablation element 12 against the wall of external carotid artery 6 as shown. An alternative mechanism for pressing ablation element 12 against the wall of external carotid artery 6 may be incorporated into carotid body modulation catheter 4, including but not limited to, an inflatable balloon, an internal pull wire configured for lateral catheter tip deflection, or an expandable structure responsive to user applied compressive force. Whichever mechanism is incorporated into carotid body modulation catheter 4 for pressing ablation element 12 against the wall of external carotid artery 6 is user actuatable by means of an actuator located in the vicinity of the proximal end of carotid body modulation catheter 4. Carotid body modulation catheter 4 may comprise a means for injecting a liquid into the carotid vasculature through a central lumen within catheter shaft 14 between a liquid port in the vicinity of distal end 27, and a liquid receiving connector in the vicinity of the proximal end. Carotid body modulation catheter 4 may be inserted into the vicinity of carotid bifurcation as using introducer sheath assembly 5 as shown, and previously described above, or without an introducer sheath by means of surgical cut-down of the superficial temporal artery and direct insertion of carotid body modulation catheter 4 into the superficial temporal artery.

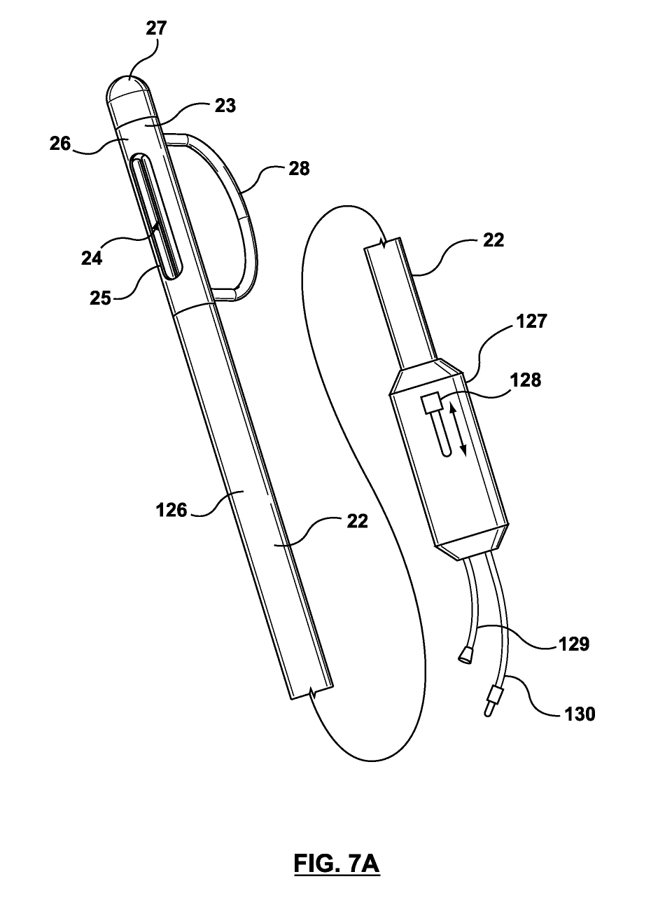

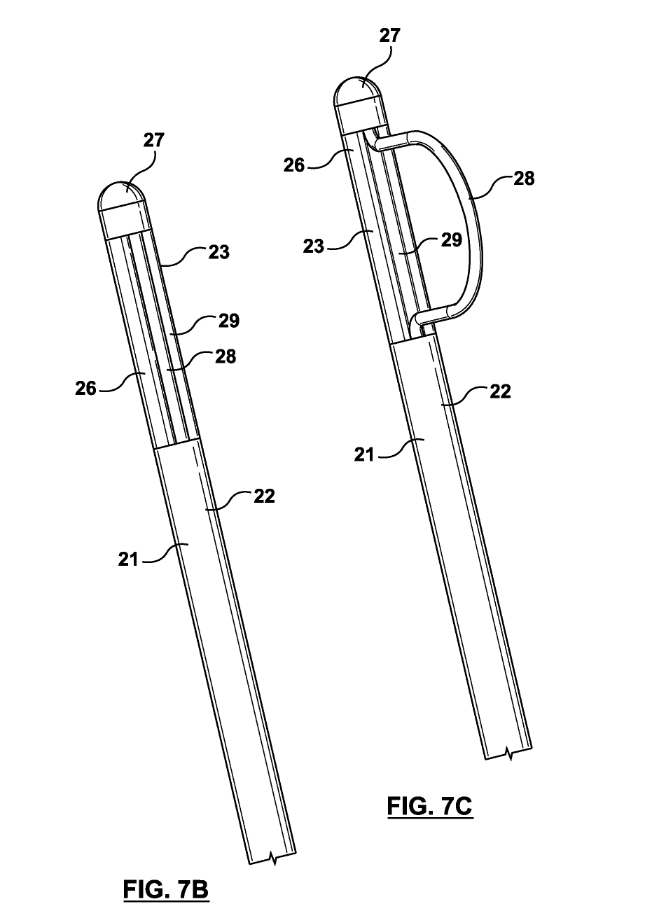

FIG. 7A is a front view illustration of the distal end of Monopolar RF Ionic Stream Carotid Body Ablation (MRF-IS-CBA) catheter 126 configured for use by superficial temporal artery access to the region of a carotid body and transmural ablation from within an external carotid artery, which utilizes an ionic liquid conduction stream. FIG. 7B is a rear view illustration of the distal end of MRF-IS-CBA catheter 126 with push wire 28 retracted. FIG. 7C is a rear view illustration of the distal end of MRF-IS-CBA catheter 126 with push wire 28 extended. MRF-IS-CBA catheter 126 comprises catheter shaft 22, RF electrode hood 23, distal tip 27, push wire 28, proximal handle 127 comprising push wire actuator 128, fluid connector 129, and RF electrical connector 130. Catheter shaft 22 is between approximately 3 French and 6 French, and is between approximately 10 cm and 25 cm long. Catheter shaft 22 comprises at least one fluid inner lumen in fluidic communication between fluid connector 129 and the interior of RF electrode hood 23, and at least one electrical conduit in electrical communication between RF electrode surface 24 disposed within the interior of RF electrode hood 23, and RF electrical connector 130. Further, catheter shaft comprises a lumen that houses push wire 28 in communication between the distal end of catheter shaft 22 and push wire actuator 128 disposed on proximal handle 127. Catheter shaft 22 is extruded from a polymeric material commonly used for vascular catheters, which may be a polyethylene, polyurethane, or nylon or other polymeric compound. Catheter shaft 22 may also have a woven, knitted, or coiled structure within its walls configured to translate rotational forces from its proximal end to its distal end to facilitated rotational positioning of electrode hood 23 within an external carotid artery. RF electrode hood 23 is a hollow cylindrical structure with a caliber approximating the caliber of catheter shaft 22, and is disposed in the vicinity of the distal end of catheter shaft 22 as shown. RF electrode hood 23 has a length between approximately 1 cm and 3 cm. RF electrode hood 23 comprises lateral fenestration 25, electrically insulated outer surface 26, and RF electrode surface 24 disposed within the interior of RF electrode hood 23 and in electrical communication with electrical connector 130 as previously described. RF electrode hood 23 may be made from an electrically conductive material, such as stainless steel, or a more precious and radiopaque material such as gold or platinum, where the insulated external surface 26 is coated with an electrically insulative material such as a polymer like PTFE, or a non-polymeric material such as a ceramic material, with the interior surface of RF electrode hood 23 comprising electrode surface 24. Alternatively, RF electrode hood 23 may be fabricated from a non-conductive polymer, and have an electrode surface disposed on the interior surface of the electrode hood. Electrode surface 24 is configured to be electrically isolated from all external surfaces of ablation catheter 126, except electrical contacts of connector 130. Lateral fenestration 25 may be one fenestration as shown, or may be more than one fenestration in lateral alignment. Lateral fenestration 25 cross sectional area is configured to be approximately less than or equal to electrode surface 24 area so that maximal current density during RF ablation is in the immediate vicinity of fenestration 25. As depicted in FIG. 7B and FIG. 7C RF electrode hood 23 comprises push wire channel 29 configured to house push wire 28 within the confides of the profile of carotid body RF ablation catheter 126. Push wire 28 comprises a wire with a diameter between approximately 0.014″ and 0.038″. Distal end of push wire 28 is fixated to distal tip 27 as illustrated. Proximal end of push wire 28 is in axial and slidable communication with push wire actuator 128 (FIG. 7A). When push wire actuator 128 advanced in the distal direction, push wire 28 extends from push wire channel to press RF electrode hood 23 and fenestration 25 against the wall of an external carotid artery adjacent to a target site (e.g., carotid body, carotid body nerves, intercarotid septum) for RF ablation of carotid body function as shown in FIG. 7C. When push wire actuator 128 is positioned in the proximal direction, push wire 28 is retracted into push wire channel 29 as shown in FIG. 7B. An alternative mechanism for pressing electrode hood 23, and fenestration 25 against the wall of external carotid artery 6 may be incorporated into carotid body modulation catheter 126, including but not limited to, an inflatable balloon, an internal pull wire configured for lateral catheter tip deflection, a deployable mesh or braid, a deployable cage, or an expandable structure responsive to user applied compressive force. Whichever mechanism is incorporated into carotid body modulation catheter 126 for pressing electrode hood 23, and fenestration 25 against the wall of external carotid artery 6 is user actuatable by means of an actuator located in the vicinity of the proximal end of carotid body modulation catheter 126. During use, an ionic liquid, such as saline is infused from fluid connector 129 through central fluid lumen not shown in catheter shaft 22, into RF electrode hood 23 and out of fenestration 25. The ionic liquid displaces arterial blood from RF electrode hood 23, and between the wall of external carotid artery 6 in contact with RF electrode hood 23, and fenestration 25, while conducting RF current between the wall of external carotid artery 6, and electrode surface 24, thereby substantially removing arterial blood from the RF electrical ablation circuit, which greatly reduces the potential for thrombotic clot formation. An indifferent electrode applied to the patient's body, and connected to the opposite pole of an RF generator as electrode surface 24 completes the RF ablation circuit. Alternatively, an indifferent electrode, also known as a dispersive electrode, may be place within the body, for example in a blood vessel such as the internal carotid artery or jugular vein.

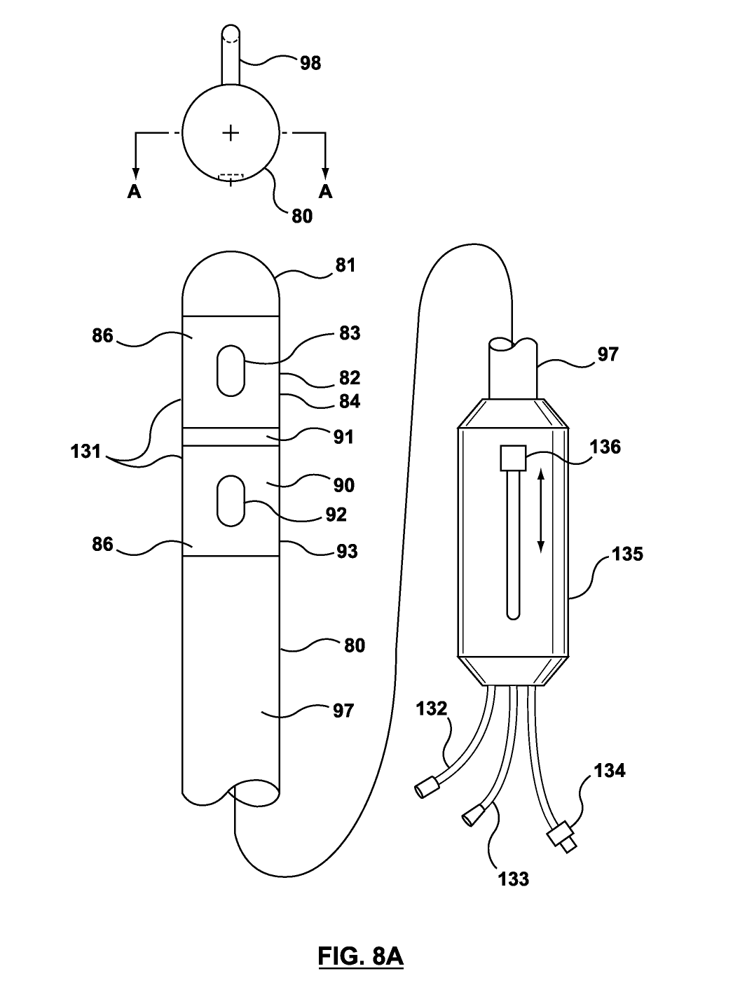

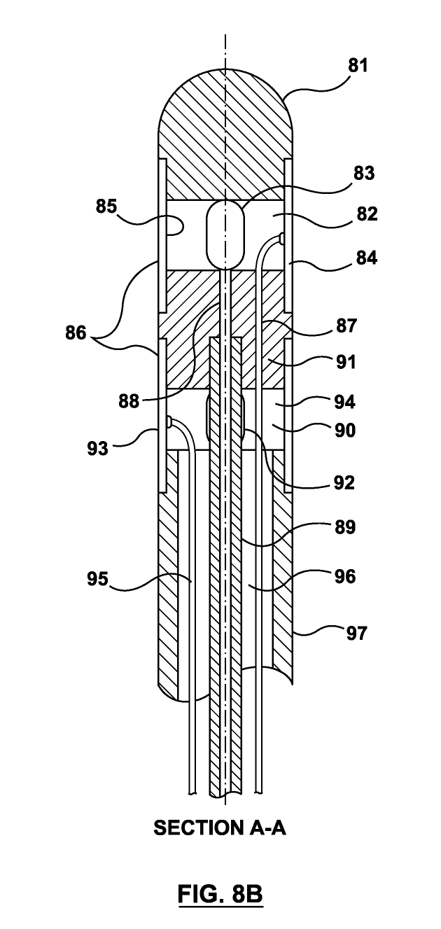

FIG. 8A is a front view illustration of a distal end of a Tandem Bipolar RF Ionic Stream Carotid Body Ablation (TBRF-IS-CBA) catheter 80 configured for use by superficial temporal artery access to a region of a carotid body and transmural RF ablation from within an external carotid artery, which utilizes dual ionic liquid conduction streams. FIG. 8B is a cross sectional illustration of the distal end of TBRF-IS-CBA catheter 80. TBRF-IS-CBA catheter 80 comprises catheter shaft 97, ablation element 131 mounted at the distal end of catheter shaft 97, distal tip 81 mounted at the distal end of ablation element 131, proximal handle 135 mounted at the proximal end of catheter shaft 97, and push wire 98 configured for pressing ablation element 131 against the wall of an external carotid artery. Catheter shaft 97 has a caliber between approximately 3 French and 6 French and a length of approximately 10 cm and 25 cm. Catheter shaft 97 comprises a central lumen 96, and at least one additional lumen for push wire 98 not shown. Catheter shaft 97 may be extruded from a polymeric material commonly used for vascular catheters, which may be a polyethylene, polyurethane, or nylon or other polymeric compound. Catheter shaft 97 may also have a woven, knitted, or coiled structure within its walls configured to translate rotational forces from its proximal end to its distal end to facilitated rotational positioning of ablation element 131 within an external carotid artery. External features (FIG. 8A) of ablation element 131 comprise distal electrode hood 82 comprising distal electrode housing 84, internal electrode surface 85 (FIG. 8B), and is associated with distal electrode fenestration 83, proximal electrode hood 90 comprising proximal electrode housing 93, internal electrode surface 94 (FIG. 8B), and is associated with proximal electrode fenestration 92. Electrode bulkhead 91 separates distal electrode hood 82 from proximal electrode hood 90. Proximal handle 135 comprises distal electrode fluid connector 132 which is in fluidic communication with the interior of distal electrode hood 82, proximal electrode fluid connector 133, which is fluidic communication with the interior of proximal electrode hood 90, RF connector 134 comprising two electrical contacts with the first contact in electrical communication with internal electrode surface 85, and with the second contact in electrical communication with internal electrode surface 94, and push wire actuator 136. Additional construction features of the distal end of TBRF-IS-CBA catheter 80 are depicted in cross section in FIG. 8B as follows: distal electrode hood 82 is defined by distal electrode hood housing 84, distal tip 81, and electrode bulkhead 91, as shown; proximal electrode hood 90 is defined by proximal electrode housing 93, electrode bulkhead 91, and catheter shaft 97. Distal electrode housing 84 and proximal electrode housing 93 are hollow thin walled cylindrical structures, and may be fabricated from a metal such as stainless steel, or a more precious metal with higher electrical conductivity and radiopacity such as a platinum or gold alloy. Distal tip 81 may be a machined or molded polymeric non-conductive structure comprising an atraumatic profile as shown. Electrode bulkhead 91 may be a machined or molded polymeric non-conductive structure as shown. All external surfaces of ablation element 131, including distal electrode housing 84, and proximal electrode housing 93 are substantially non-conductive, which may comprise a non-conductive polymeric coating 86 such as PTFE, polyimide, of polyethylene, or may be a non-polymeric coating such as a ceramic coating. Ablation element assembly 131 may be assembled as shown using adhesives. Electrical conductor 87 is in electrical communication with distal electrode surface 85, and one contact of bipolar electrical connector 134. Electrical conductor 95 is in electrical communication with distal electrode surface 85, and a second contact of bipolar electrical connector 134. Both electrical conductors 87 and 95 are coated with an electrical insulator such that there is substantially no electrical communication between distal electrode surface 85 and proximal electrode surface 94 in the presence of an ionic liquid within central lumen 96, interior of distal electrode hood 82, and interior of proximal electrode hood 90. Distal fluid tube 89 provides fluidic communication between distal fluid connector 132 and the interior of distal electrode hood 82. Central lumen 96 provides fluidic communication between proximal fluid connector 133 and the interior of proximal electrode hood 93. Distal fenestration 83 and proximal fenestration 92 are substantially laterally aligned with an axial length between approximately 3 mm and 7 mm, with a width of between approximately 0.5 mm and 1.2 mm. Push wire 98 is disposed substantially at the level of ablation element 131 and is configured to push lateral fenestrations 83 and 92 against the wall of an external carotid artery adjacent to a target site (e.g., carotid body, carotid body nerves, intercarotid septum). The function and construction of push wire 98 is similar to the function and construction of push wire 28 described in FIGS. 7A-7C. An alternative mechanism for pressing ablation element 131, and fenestrations 83 and 92 against the wall of external carotid artery 6 may be incorporated into TBRF-IS-CBA catheter 80, including but not limited to, an inflatable balloon, an internal pull wire configured for lateral catheter tip deflection, a deployable mesh, braid, or cage, or an expandable structure responsive to user applied compressive force. Whichever mechanism is incorporated into TBRF-IS-CBA catheter 80 for pressing ablation element 131 and fenestrations 83 and 92 against the wall of external carotid artery 6, it is user actuatable by means of an actuator located in the vicinity of the proximal end of TBRF-IS-CBA catheter 80. During use, an ionic liquid, such as saline is infused from distal fluid connector 132 through distal fluid tube 89 shown within in catheter shaft 22, into distal electrode hood 82 and out of fenestration 83. A separate source of ionic liquid is infused from proximal fluid connector 133, central lumen 96 into proximal electrode hood 90 and out proximal fenestration 92. The ionic liquid displaces arterial blood from distal electrode hood 82 and proximal electrode hood 90, and between the wall of external carotid artery 6 in contact with ablation element 131, while conducting RF current between the wall of external carotid artery 6, and distal electrode surface 85, and between the wall of external carotid artery 6 and proximal electrode surface 94, thereby substantially removing arterial blood from the RF electrical ablation circuit, which greatly reduces the potential for thrombotic clot formation. By using the tandem bipolar RF electrode configuration disclosed hear in, RF current, and associated joule effect heating is localized to the immediate vicinity of the two RF electrodes, preventing distant adverse thermal effects.

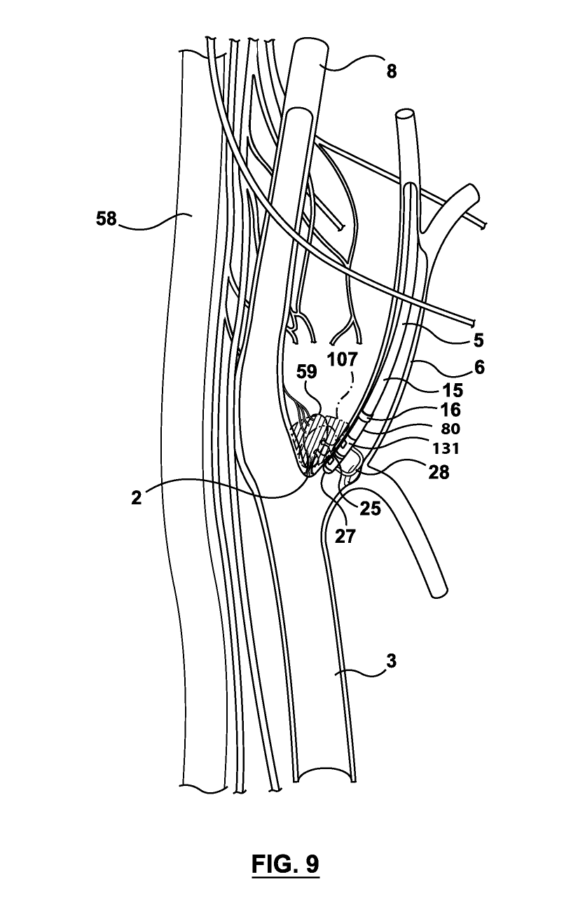

FIG. 9 is a schematic illustration of TBRF-IS-CBA catheter 80, (and may represent carotid body modulation catheter 126 used in a similar manner), in situ, with access to the region of the carotid body from a superficial temporal artery puncture. Ablation element 131 is being pushed against the wall of external carotid artery 6 immediately distal to carotid bifurcation 2 and immediately adjacent to carotid body 59. TBRF-IS-CBA catheter 80 may be inserted into the vicinity of carotid bifurcation as using introducer sheath assembly 5 as shown, and previously described above, or without an introducer sheath by means of surgical cut-down of the superficial temporal artery and direct insertion of carotid body modulation catheter 4 into the superficial temporal artery. Ablation element 131 is advanced to the level of carotid body 59 under radiographic guidance. The radial position of fenestrations 83 and 92 are determined by injection of a radiographic contrast medium through fluid connector 132 or fluid connector 133 which exits fenestration 83 or fenestration 92 giving the user a fluoroscopic indication of the radial position of fenestrations 83 and 92. Catheter shaft is 97 is then rotated to radially position fenestrations 83 and 92 towards carotid body 59. Push wire 98 is then extended using actuator 136 pressing ablation element 131 and fenestrations 83 and 92 against the wall of external carotid artery 6. Fluid connectors 132 and 133 are connected to separate sources of ionic liquid, not shown, which may be a syringe filled with saline configured for use with a syringe pump, or may be a bag of saline elevated and flow motivated by gravity or a pressure cuff. Bipolar electrical connector 134 is connected to an RF generator, not shown. RF generator ablation parameters are selected, which may comprise a power setting between approximately 2 and 20 watts, and a time between approximately 10 and 120 seconds. Saline flow is then initiated displacing blood from electrode hoods 82 and 90, and between fenestrations 83 and 92 and the wall of external carotid artery 6. The RF generator is then activated to apply RF current between electrode surfaces 85 and 94 conducted through the saline and the wall and periarterial tissue between fenestrations 83 and 92 resulting in ablation zone 107 comprising carotid body 59. Upon completion of the RF energy application, carotid body function may then be assessed. If it determined that carotid body function is above a determined clinical threshold, then the ablation may be repeated at the same or different RF ablation parameters.

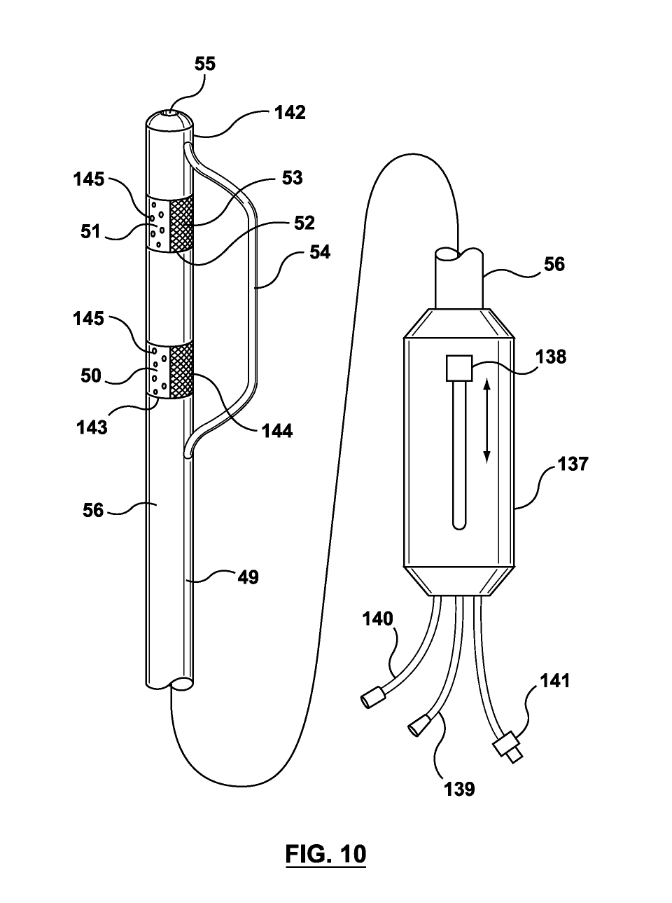

FIG. 10 is an illustration of a lateral tandem bipolar RF carotid body ablation (LTB-RF-CBA) catheter 49. LTB-RF-CBA catheter 49 comprises catheter shaft 56, proximal lateral electrode 50, distal lateral electrode 51, push wire 54, central lumen 55, and proximal handle, not shown. Catheter shaft 56 has a caliber between approximately 3 French and 6 French and a length of approximately 10 cm to 25 cm. Catheter shaft 56 comprises a central lumen 55, and at least one additional lumen for push wire 54 not shown. Catheter shaft 56 is extruded from a polymeric material commonly used for vascular catheters, which may be a polyethylene, polyurethane, or nylon or other polymeric compound. Catheter shaft 56 may also have a woven, knitted, or coiled structure within its walls configured to translate rotational forces from its proximal end to its distal end to facilitated rotational positioning of lateral electrodes 50 and 51 within an external carotid artery. Central lumen 55 is in fluidic communication between the distal end 142 of catheter shaft 56 as shown and fluid connector 139, which may be a female luer connector at the proximal end. Central lumen 55 may be configured for use with a guide wire between approximately 0.014″ to 0.038″ in diameter. Central lumen 55 also provides a means for injecting a radiographic or ultrasonic contrast medium into the carotid vasculature for imaging of carotid vasculature for assisted positioning of ablation electrodes 50 and 51 against the wall of an external carotid artery adjacent to a target site. Central lumen 55 may deposit contrast fluid from a port positioned at a distal region of the catheter more than approximately 15 mm (e.g., 15 to 20 mm, 15 to 30 mm, 15 to 40 mm) distal to electrode 50. In such an arrangement contrast may be deposited in a common carotid artery 3 and carried by blood flow to both an internal carotid artery 8 and an external carotid artery 6 so the carotid bifurcation 2 can be imaged and placement of electrodes 50 and 51 relative to the bifurcation can be determined. Optionally catheter shaft 56 may comprise a fluid lumen between irrigation fluid connector 140 and at least one fluid port(s) 145 in the vicinity of lateral electrodes 50 and 51 which may be used for irrigating the surface of electrodes 50 and 51 for the purpose cooling, or preventing thrombus formation on electrodes 50 and 51. Fluid port(s) 145 may comprise multiple micro-drilled holes in electrode surfaces 50 and 51, as shown, in communication with irrigation lumen, not shown, and irrigation fluid connector 140. Proximal electrode surface 50 is an exposed surface of proximal electrode ring 143. Electrically insulated surface 144 comprises an electrically insulative coating of proximal electrode ring 143. Distal electrode surface 51 is an exposed surface of distal electrode ring 52. Distal insulated surface 53 comprises an electrically insulative coating of distal electrode ring 52. Insulative coatings 53 and 144 may be polymeric coating such as PTFE, polyethylene, polyurethane of polyimide, of may be a non-polymeric coating such as a ceramic coating. Electrode surfaces 50 and 51 are in lateral alignment as shown, and are configured to be diametrically opposed to push wire 54 as shown. Optionally, electrode rings 52 and 143 may be devoid of insulative coatings 53 and 144 resulting in electrode surfaces 50 and 51 being circumferential electrode surfaces. Electrode rings 143 and 52 comprise a metallic thin walled cylindrical ring disposed on the outer surface of catheter shaft 56. Distal electrode ring 52 is connected to one electrical contact in bipolar RF connector 141 by an electrical conduit within catheter shaft 56. Proximal electrode ring 143 is connected to a second electrical contact in bipolar RF connector 141 by a second electrical conduit within catheter shaft 56. Bipolar RF connector 141 is configured to connect distal electrode ring 52 to one pole of an RF generator, and proximal electrode ring 143 to the second pole of an RF generator. Push wire 54 is a flexible wire, which may comprise a coiled wire around a central wire with construction similar to a guide wire or may comprise an electrically non-conductive material. Distal end of push wire 54 is fixated within the vicinity of distal tip 142. The proximal end of push wire 54 is connected to push wire actuator 138, which is disposed on proximal handle 137. Pushing the push wire actuator in the distal direction extends push wire 54 in a lateral direction opposite electrode surfaces 50 and 51. Moving push wire actuator in the proximal direction retracts push wire 54 in an intimate position with catheter shaft 56. Push wire 54 is radiopaque, and provides the user with an unambiguous fluoroscopic indication of the radial position of the distal end of LTB-RF-CBA catheter 49 within the carotid vasculature. Push wire 54 is used to push electrode surfaces 50 and 51 against the wall of an external carotid artery, however, there are alternative mechanisms that could be used, including using an internal pull wire to laterally deflect the electrode surfaces 50 and 51 against the wall of an external carotid artery adjacent to a target site (e.g., carotid body, carotid body nerves, intercarotid septum), or another mechanism may be used.

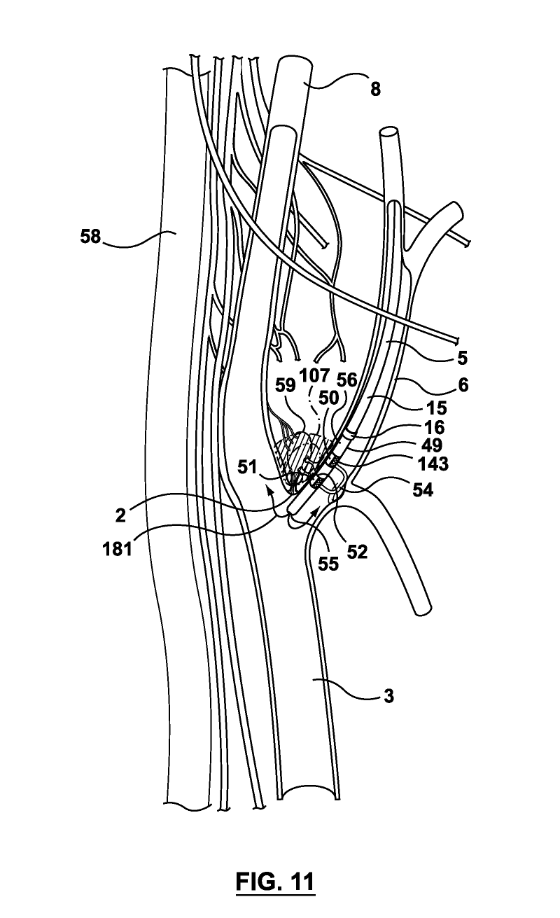

FIG. 11 is a schematic illustration of a carotid LTB-RF-CBA catheter 49 in situ, with access to the region of the carotid body 59 from a superficial temporal artery puncture. Electrode surfaces 50 and 51 are being pushed against the wall of external carotid artery 6 cranial to carotid bifurcation 2 and adjacent to a target site (e.g., carotid body 59). LTB-RF-CBA catheter 49 may be inserted into a vicinity of carotid bifurcation 2 using introducer sheath assembly 5 as shown, and previously described above, or without an introducer sheath by means of surgical cut-down of the superficial temporal artery and direct insertion of LTB-RF-CBA catheter 49 into the superficial temporal artery. Electrode surfaces 50 and 51 are advanced to the level of carotid body 59 under radiographic or ultrasonic guidance. Contrast 181 is shown being deposited into the blood stream caudal to carotid bifurcation 2 so it may flow in to internal carotid artery 8 and external carotid artery 6. The radial position of electrode surfaces are determined by extending and retracting radiopaque push wire 54 giving the user a fluoroscopic indication of the radial position of surfaces 50 and 51. Catheter shaft 56 is then rotated to radially position electrode surfaces 50 and 51 towards carotid body 59. Push wire 54 is then extended using actuator 138 pressing electrode surfaces 50 and 51 against the wall of external carotid artery 6. An irrigation fluid connector is connected to a source of ionic liquid, not shown, which for example may be a syringe filled with saline configured for use with a syringe pump, or may be a bag of saline elevated and flow motivated by gravity or a pressure cuff. Bipolar electrical connector 141 is connected to an RF generator, not shown. RF generator ablation parameters are selected, which may comprise a power setting between approximately 2 and 20 watts, and a time between approximately 10 and 120 seconds. Saline flow is then initiated displacing blood from electrode surfaces 50 and 51, and cooling electrode surfaces 50 and 51. The RF generator is then activated to apply RF current between electrode surfaces 50 and 51 conducted through the saline and the wall and periarterial tissue between electrode surfaces 50 and 51 resulting in ablation zone 107 comprising carotid body 59. Upon completion of the RF energy application, carotid body function may then be assessed. If it is determined that carotid body function is above a determined clinical threshold, then the ablation may be repeated at the same or different RF ablation parameters.

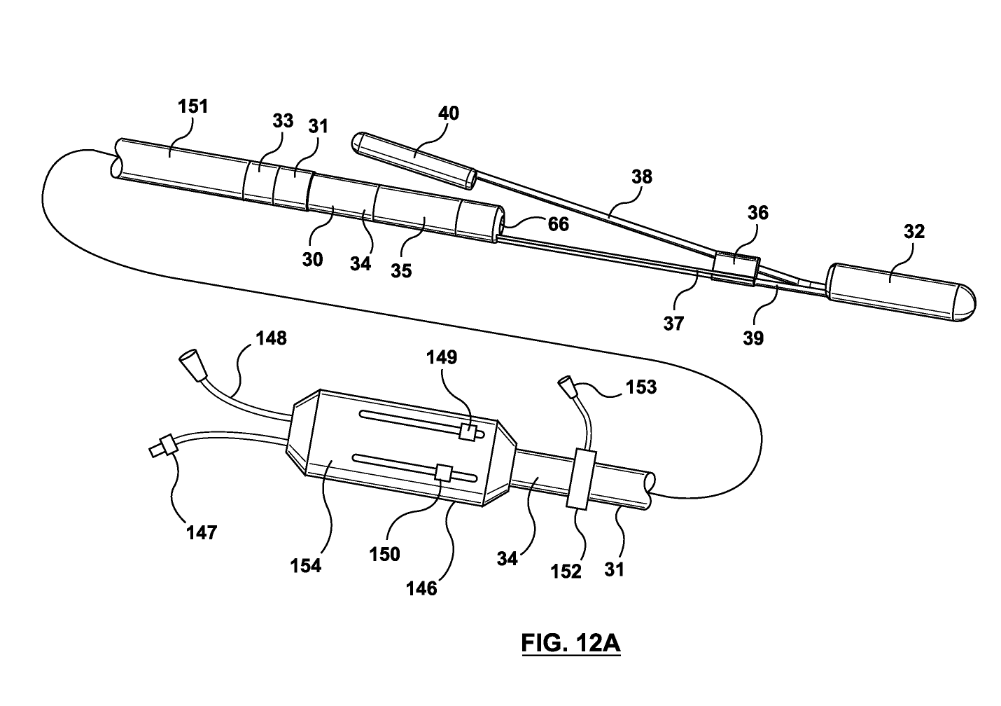

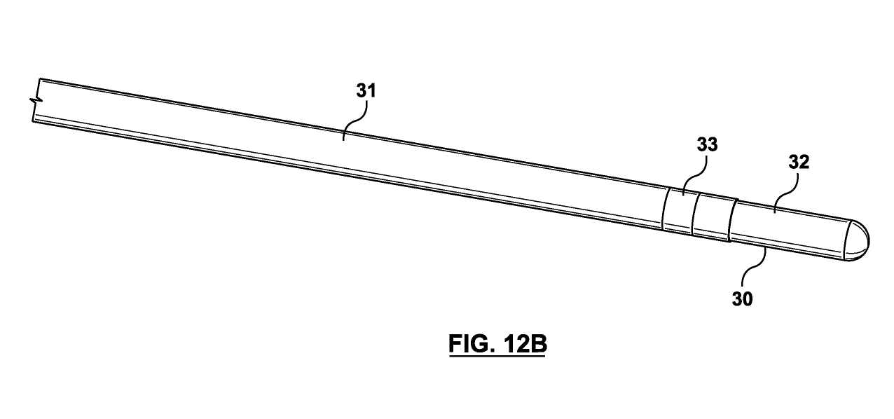

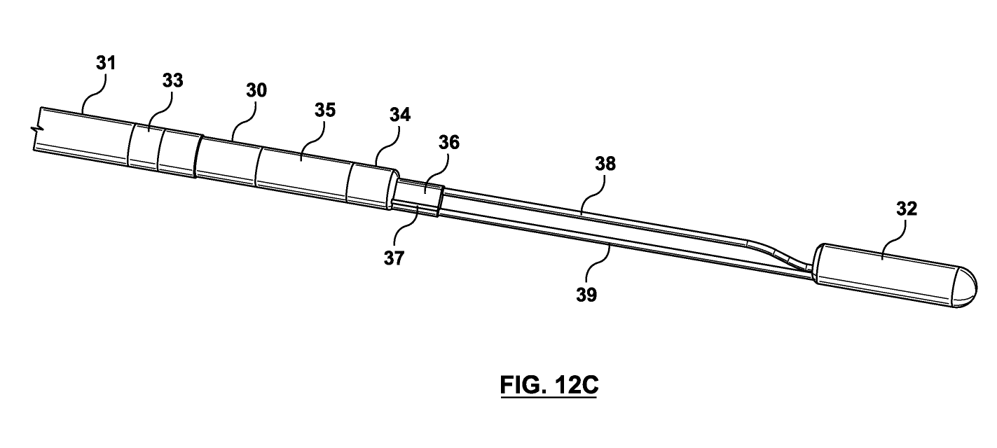

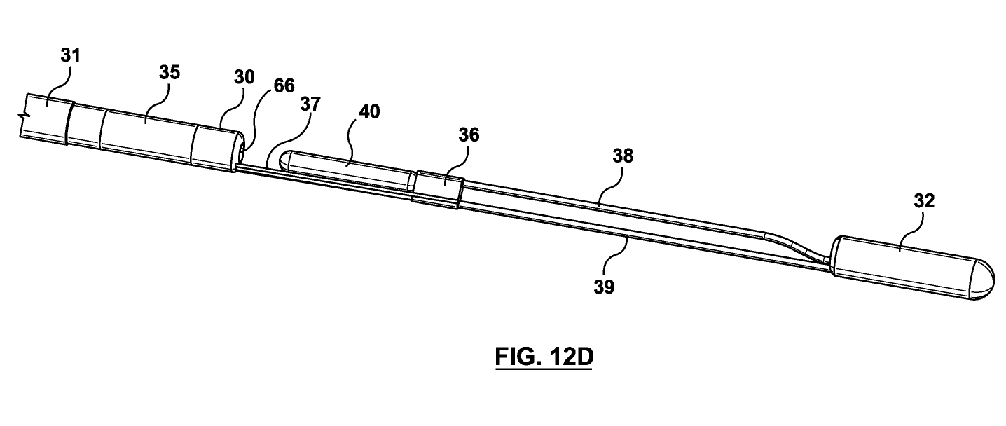

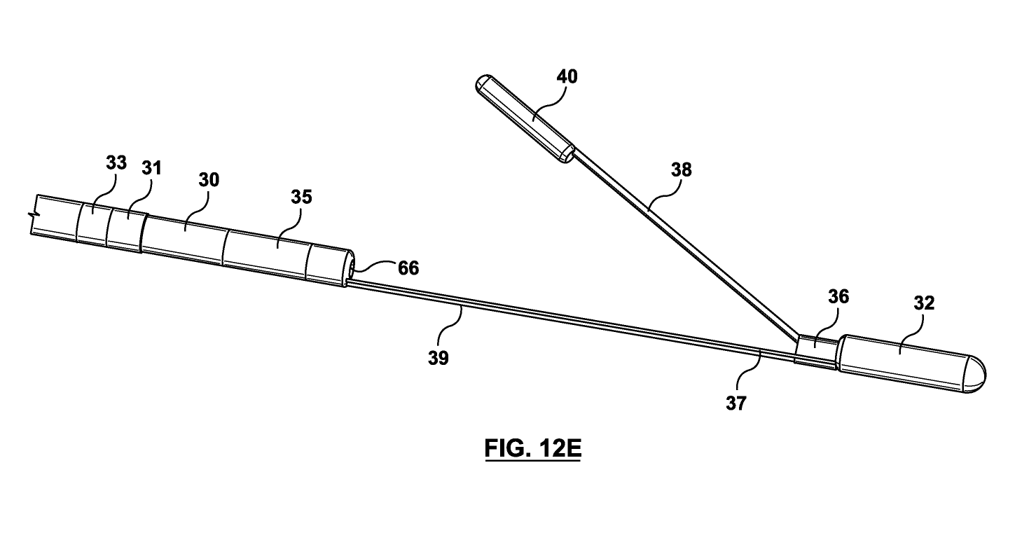

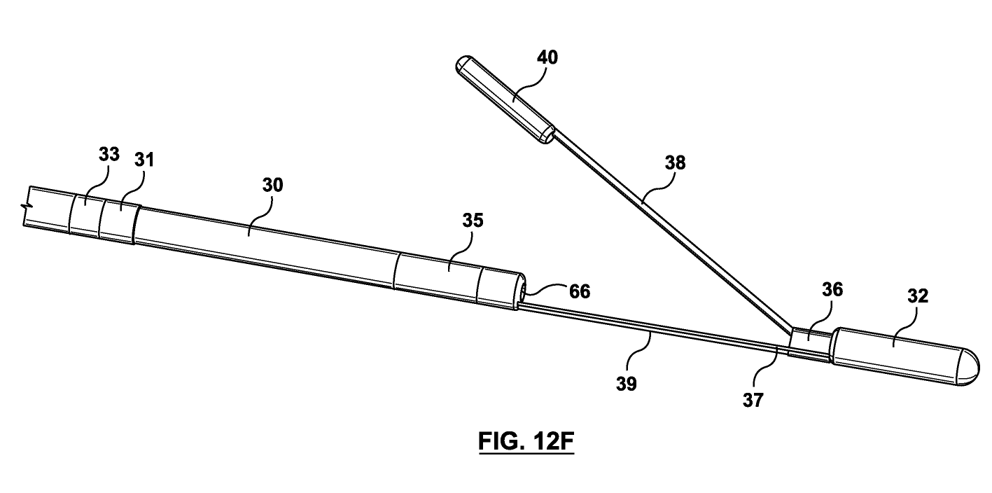

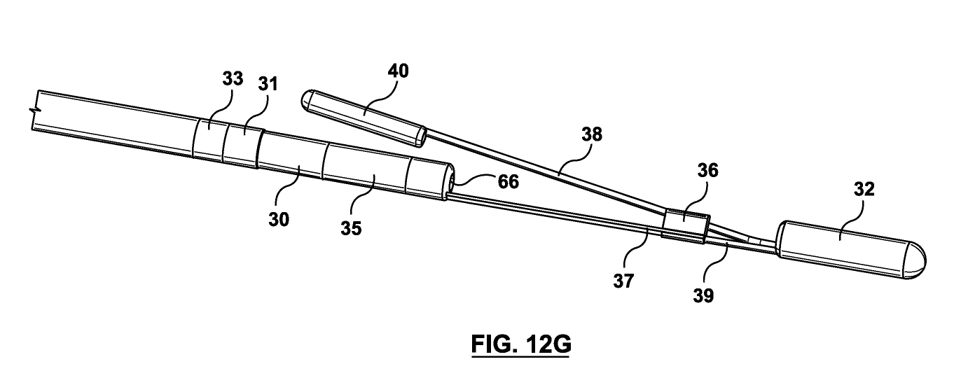

FIG. 12A is an illustration of Retrograde Carotid Body Ablation Bipolar (R-CBA-B) catheter 30. FIG. 12B is an illustration of the distal end of R-CBA-B catheter 30 in its superficial temporal artery insertion configuration. FIG. 12C is an illustration of the distal end of R-CBA-B catheter 30 with the outer sheath 31 retracted. FIG. 12D is an illustration of the distal end of R-CBA-B catheter 30 shown with the bifurcation coupling electrode 40 withdrawn for the catheter shaft central lumen 66 for bifurcation coupling arm 38 deployment. FIG. 12E is an illustration of the distal end of a R-CBA-B catheter 30 shown with the bifurcation coupling actuator clasp 36 and control wire 37 in the maximal distal position with the bifurcation coupling arm 38 in its pre-biased lateral position. FIG. 12F is an illustration of the distal end of R-CBA-B catheter 30 shown with catheter shaft 34 advanced in the distal direction with the shaft ring electrode 35 positioned in opposition to the bifurcation coupling arm electrode 40 for bipolar ablation of the carotid bifurcation saddle. FIG. 12G is an illustration of the distal end of R-CBA-B catheter 30 shown with the bifurcation coupling actuator clasp 36 and control wire 37 pulled in the proximal direction to apply a pinching force to the carotid bifurcation saddle. R-CBA-B catheter 30 comprises catheter shaft 34, distal tip 32, distal tip actuation wire 39, bifurcation coupling electrode 40, bifurcation coupling arm 38, bifurcation coupling actuator clasp 36, bifurcation coupling actuator clasp wire 37, ring electrode 35, outer sheath 31, and proximal handle assembly 146. Proximal handle assembly comprises handle 154, distal tip actuator 149, bifurcation coupling actuator 150, fluid connector 148, and bipolar RF connector 147. Outer sheath comprises sheath tube 151, sheath valve 152, and distal RO marker 33. Catheter shaft 34 is between approximately 4 French and 6 French, and is between approximately 10 cm and 25 cm long. Catheter shaft 34 comprises central lumen 66 in fluidic communication between fluid connector 149 and the distal end of catheter shaft 34, which is also configured to house bifurcation coupling electrode 40 and bifurcation coupling arm 38 during insertion and removal, and at least one electrical conduit in electrical communication between ring electrode 35 and bipolar RF connector 147. Further, catheter shaft 34 comprises a lumen in slidable relationship with distal tip actuation wire 39, and an additional lumen in slidable relationship with bifurcation coupling actuator clasp wire 37. Catheter shaft 34 is extruded from a polymeric material commonly used for vascular catheters, which may be a polyethylene, polyurethane, or nylon or other polymeric compound. Catheter shaft 34 may also have a woven, knitted, or coiled structure within its walls configured to translate rotational forces from its proximal end to its distal end to facilitated rotational positioning of bifurcation coupling electrode 40 within the carotid vasculature. Distal tip 32 may be a bullet-shaped polymeric structure with approximately the same caliber as catheter shaft 34. Distal tip 32 may be insert molded over distal tip actuation wire 39 and bifurcation coupling arm wire 38. An electrical connection is made between distal tip actuation wire 39 and bifurcation coupling arm wire 37 within distal tip 32. Distal tip 32 may also comprise a radiopaque marker, not shown, to provide the user with fluoroscopic image of the position of distal tip 32 within the carotid vasculature. Distal tip actuation wire 39 is connected to distal tip 32 at the distal end, and is housed within a lumen within catheter shaft 34, not shown, in a slidable relationship, and is connected to distal tip actuator 149 of proximal handle assembly 146, and is in electrical communication with one contact within bipolar RF connector 147. Distal actuator wire is approximately 0.010″ to 0.030″ in diameter, and may be a super elastic nickel-titanium alloy. The length of distal tip actuation wire 39 is configured so that distal tip actuation length is between approximately 3 cm and 10 cm. Distal tip actuation wire 39 comprises an electrically insulative coating, which may comprise polymeric coating such as PTFE, polyethylene, polyurethane, polyimide, or another polymeric coating, or may be a non-polymeric coating such as a ceramic coating. Bifurcation coupling electrode 40 is approximately 1 mm in diameter and between approximately 4 mm and 10 mm long. Bifurcation coupling electrode may be machined of a metal alloy with high thermal conductivity and radiopacity such as a gold or platinum, or may be made from a less noble metal alloy such as stainless steel. Bifurcation coupling electrode 40 is connected to bifurcation coupling arm 38 by soldering or welding providing an electrical connection between bifurcation coupling arm 38 and bifurcation coupling electrode 40. Bifurcation coupling arm 38 comprises a wire with a pre-formed shape configured for a lateral positioning bias of bifurcation coupling electrode 40 as shown in FIG. 12E. Arm 38 has a wire diameter between approximately 0.010″ and 0.030″, and may be a superelastic nickel-titanium alloy. The length of bifurcation coupling arm 38 is configured so that the distance between distal tip 32 and bifurcation coupling electrode is between approximately 2 cm and 4 cm. Bifurcation coupling arm 38 is electrically connected to distal tip actuation wire 39 within distal tip 32. Bifurcation coupling arm wire 38 comprises an electrically insulative coating, which may comprise polymeric coating such as PTFE, polyethylene, polyurethane, polyimide, or another polymeric coating, or may be a non-polymeric coating such as a ceramic coating. Bifurcation coupling actuator clasp 36 is bonded to the distal end of bifurcation coupling actuator wire 37 and is in a slidable relationship over bifurcation coupling arm 38, and distal tip actuator wire 39 as shown. Bifurcation coupling actuator clasp may fabricated from a polymeric material, or a metallic material. Clasp actuator wire 37 is connected to bifurcation coupling actuator clasp at the distal end, and bifurcation coupling arm actuator 150 of proximal handle assembly 146, and resides within a lumen of catheter shaft 34 in a slidable relationship with the lumen, not shown. Bifurcation coupling actuator wire 37 is between approximately 0.010″ and 0.030″ in diameter, and its length is configured to fully arrest the lateral pre-formed bias in bifurcation coupling arm 38 in one maximal actuated position, and to substantially not inhibit lateral bias of bifurcation coupling arm 38 in its opposite maximal actuated position as shown within these figures. Bifurcation coupling actuator wire may be fabricated from a super-elastic nickel-titanium alloy. Ring electrode 35 is disposed on the surface of catheter shaft 34 in the vicinity of the distal end. Ring electrode 35 has an outer diameter approximately as catheter shaft 34, and has a wall thickness between approximately 0.002″ and 0.006″. Ring electrode 35 is connected to a second contact of bipolar RF connector 147 by an electrical wire residing within catheter shaft 34. Ring electrode 35 may be fabricated from an alloy with high thermal conductivity and high radiopacity such as a gold or platinum alloy. Outer sheath 31 may be introducer sheath 5 as described above, or a dedicated sheath as part of R-CBA-B catheter 30 assembly. Outer sheath 31 comprises sheath tube 151 comprising a thin walled hollow structure, sheath valve 152, fluid connector 153 and radiopaque marker 33. Sheath tube 151 has an inner diameter sized to house catheter shaft 34 and distal tip 32 and has a working length of approximately 5 cm to 20 cm, with the working length being the distance from the distal end and sheath valve 152. Sheath tube 151 is configured for R-CBA-B catheter 30 where the inner diameter of sheath tube 151 is a fraction of a millimeter larger than the outside diameter of catheter shaft 34 and distal tip 32. Sheath tube 151 has a wall thickness between approximately 0.25 mm and 0.75 mm, and is an extrusion of a flexible polymeric material, which may be a polyurethane, polyethylene or other polymeric compound typically used in vascular catheter and sheath construction. Radiopaque marker 33 is bonded to sheath tube 151 in the vicinity of its distal tip and comprises a thin walled ring of radiopaque metal, or a paint comprising a radiopaque metal. Sheath valve 152 comprises an elastomeric valve configured to prevent blood from exiting the sheath when inserted into a superficial temporal artery with or without R-CBA-B catheter 30 inserted into outer sheath 31. Those skilled in the art of sheath construction are familiar with sheath valve design and construction, therefore, no further description is warranted. Fluid connector 153 is in fluidic communication with the inner lumen of sheath tube 151, and is used to insert and remove fluid from outer sheath 31. Handle 154 is ergonomically designed so the user may comfortably hold the handle, maneuver R-CBA-B catheter 30 axially and rotationally, and manipulate distal tip actuator 149, and bifurcation coupling actuator 150. As shown, distal tip actuator is a sliding mechanism where in the forward or distal position distal tip 32 is extended, and in the backwards or proximal position distal tip 32 is retracted. As shown, bifurcation coupling actuator 150 is a sliding mechanism where in the forward or distal position bifurcation coupling actuator clasp 36 is extended, and in the backwards or proximal position bifurcation coupling actuator clasp is retracted. Bipolar RF connector 147 is configured to connect bifurcation coupling electrode 40 to one pole of an RF generator, not shown, and ring electrode 35 to the second pole of the RF generator. Fluid connector 148 is in fluidic communication with central lumen 66 and configured with a female luer fitting to facilitate connection to a syringe or other common fluid sources.

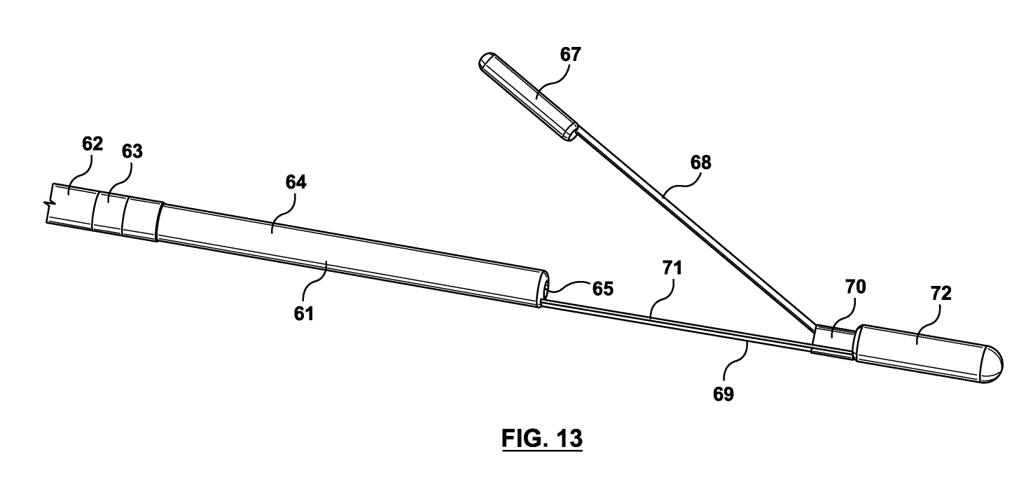

FIG. 13 is an illustration of the distal end of a Retrograde Carotid Body Monopolar Deployable Arm (R-CBA-MDA) catheter 61 configured for use by superficial temporal artery access to the region of a carotid body. R-CBA-MDA catheter 61 is similar to R-CBA-B catheter 31 described above, except, R-CBA-MDA catheter 61 is void of a ring electrode, and instead uses an indifferent, or dispersive electrode (not shown) to complete the RF circuit with the electrode 67. The following components are depicted, and have similar form and functionality to the corresponding components with the R-CBA-B catheter 30 described above: catheter shaft 64, central lumen 65, distal tip actuation wire 69, deployable arm 68, electrode 67, actuator clasp 70, actuator clasp wire 71, outer sheath 62, and radiopaque marker 63.

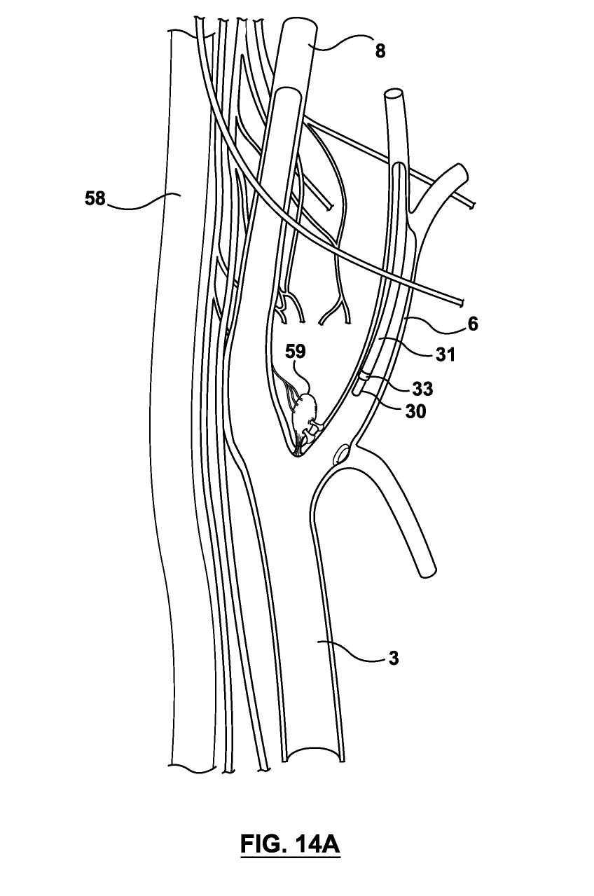

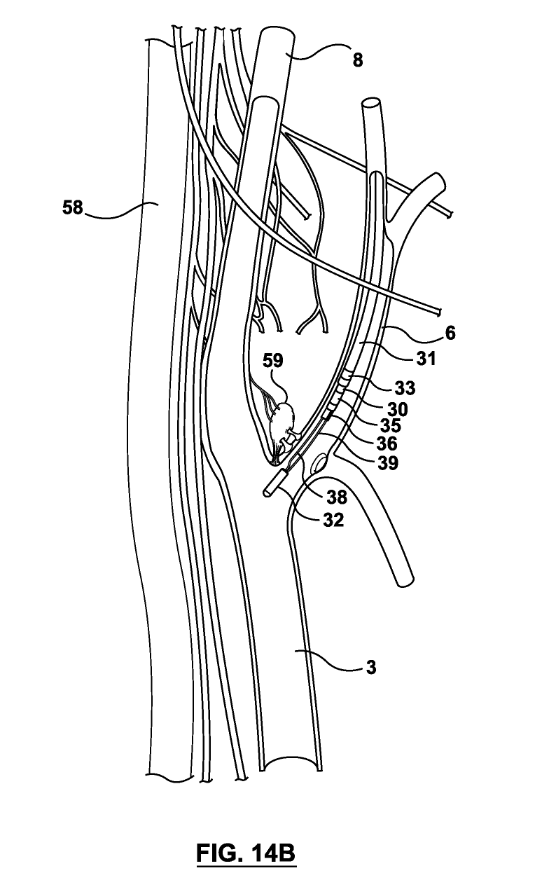

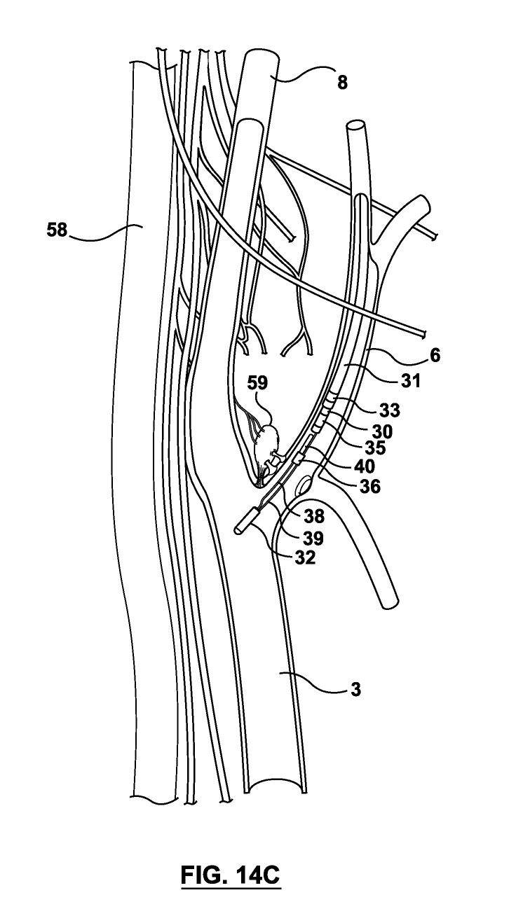

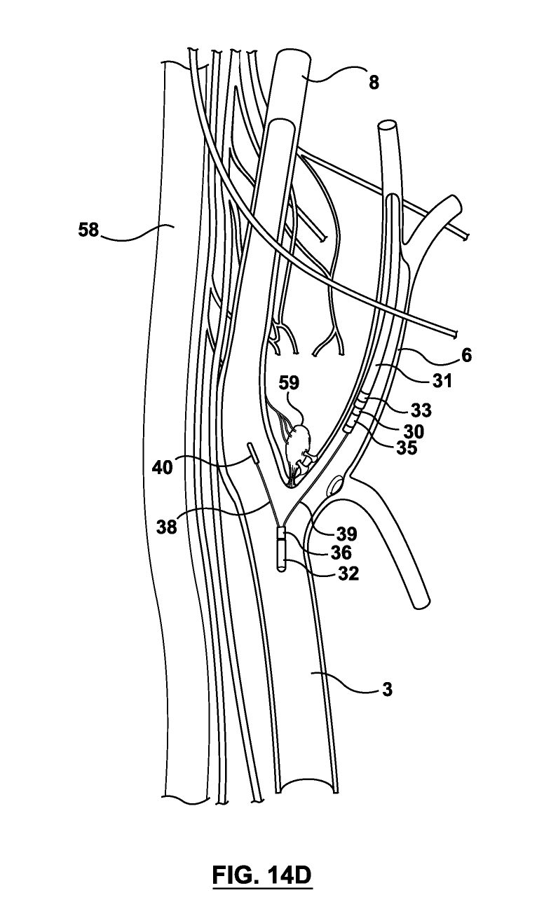

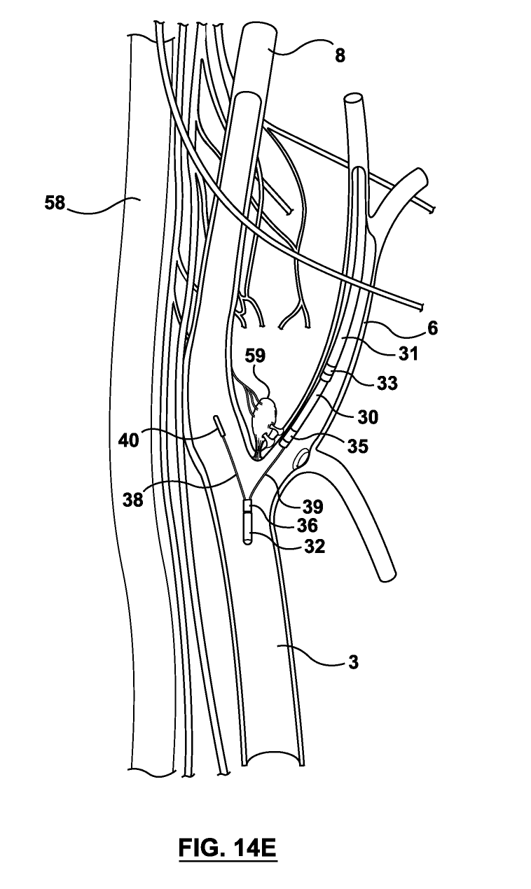

FIG. 14A is an in situ schematic illustration of the distal end of a R-CBA-B catheter 30 configured for use by trans-superficial temporal artery access to the region of a carotid body shown in its insertion configuration. FIG. 14B is an in situ schematic illustration of the distal end of R-CBA-B catheter 30 shown with the outer sheath 31 retracted exposing ring electrode 35, bifurcation coupling arm 38, distal tip 32, actuator clasp 36 and actuator clasp wire 37, with the bifurcation coupling electrode 40 docked within the central lumen 66 of the catheter shaft 34. FIG. 14C is an in situ schematic illustration of the distal end of R-CBA-B catheter 30 shown with the bifurcation coupling electrode 40 withdrawn from the catheter shaft central lumen for bifurcation coupling arm deployment. FIG. 14D is an in situ schematic illustration of the distal end of R-CBA-B catheter 30 shown with the bifurcation coupling actuator clasp 36 and actuator clasp wire 37 in the maximal distal position with the bifurcation coupling arm 38 in its pre-formed biased position. FIG. 14E is an in situ schematic illustration of the distal end of R-CBA-B catheter 30 shown with catheter shaft 34 advanced in the distal direction with the ring electrode 35 positioned in opposition to bifurcation coupling electrode 40 for bipolar ablation of carotid body 59. FIG. 14F is an in situ schematic illustration of the distal end R-CBA-B catheter 30 shown with the bifurcation coupling actuator clasp 36 and actuator clasp wire 37 pulled in the proximal direction to apply a pinching force to the carotid bifurcation 2. Bipolar electrical connector 147 is then connected to an RF generator, RF ablation parameters are selected, and RF ablation energy is applied forming ablation zone 107 comprising carotid body 59.

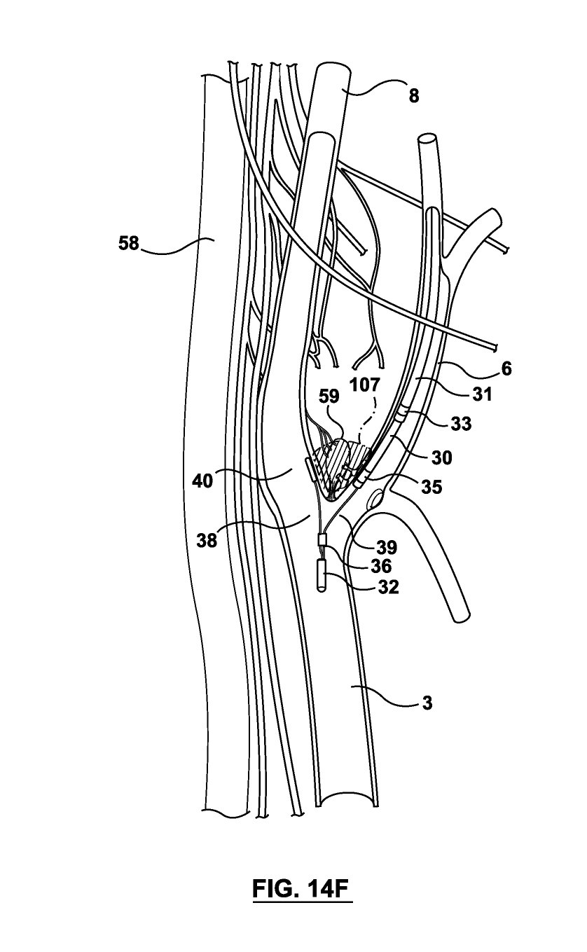

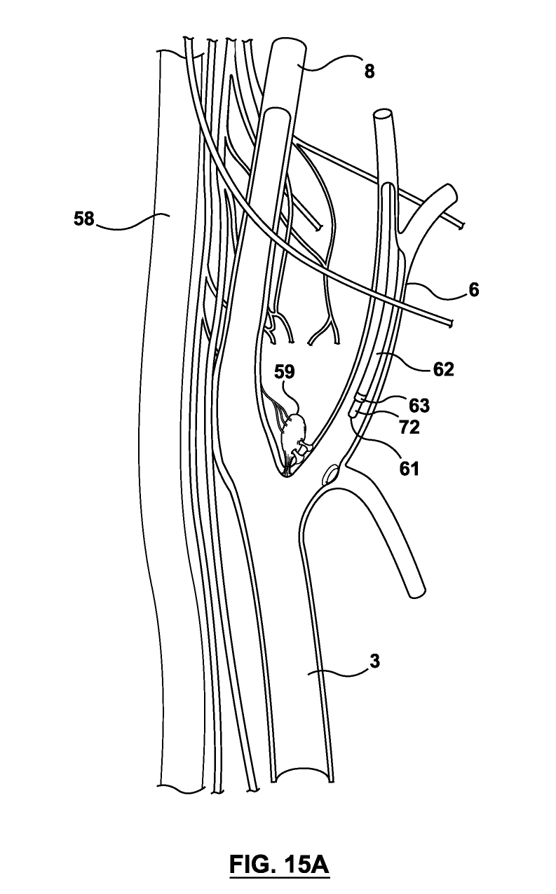

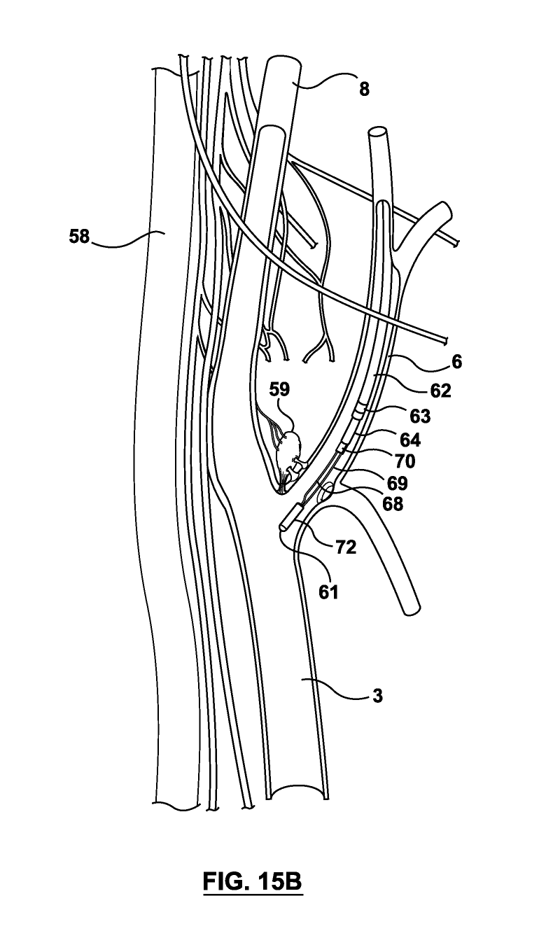

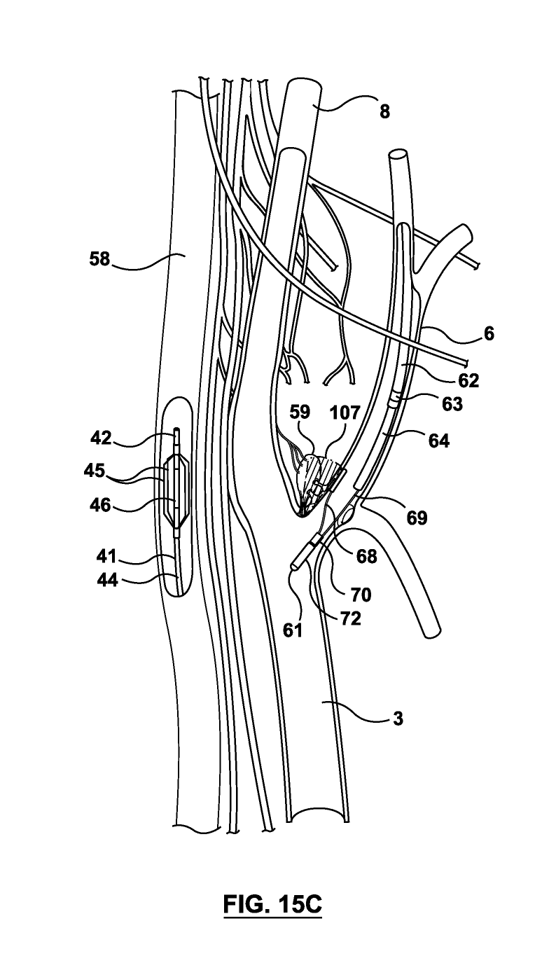

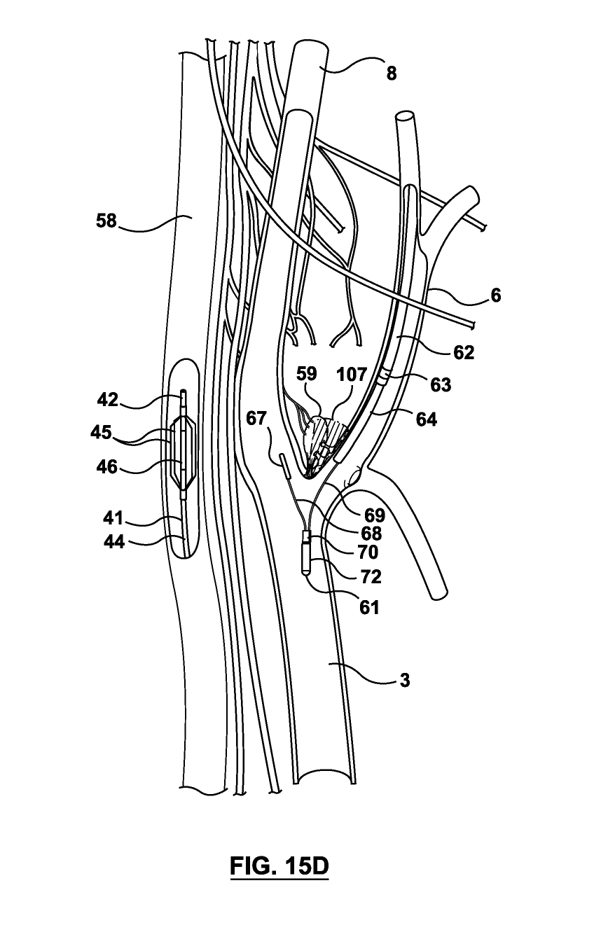

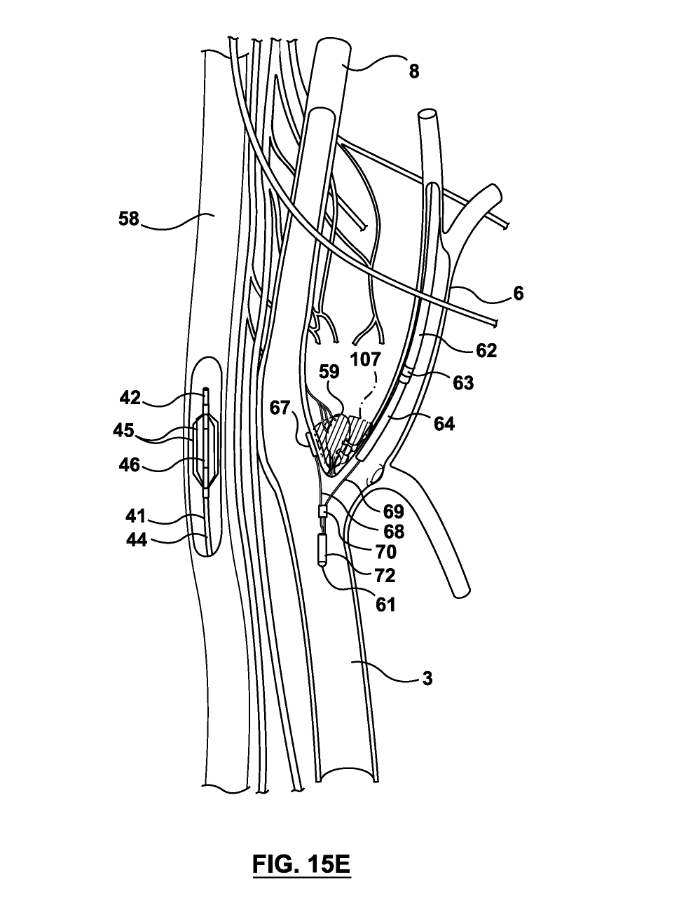

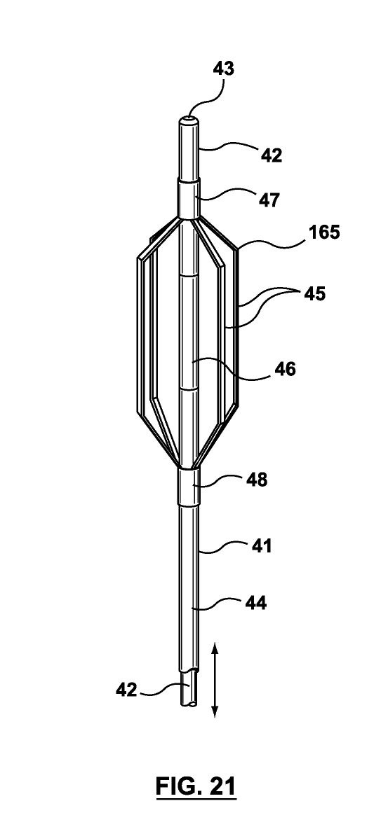

FIG. 15A is an in situ schematic illustration of the distal end of a R-CBA-MDA catheter 61 configured for use by trans-superficial temporal artery access to the region of a carotid body shown in its insertion configuration. FIG. 15B is an in situ schematic illustration of the distal end of R-CBA-MDA catheter 61 shown with the outer sheath 62 retracted exposing deployable arm 68, distal tip 72, and actuator clasp 70 and actuator clasp wire 71, with the bifurcation coupling electrode 67 docked within the central lumen 65 of the catheter shaft 64. FIG. 15C is an in situ schematic illustration of the distal end of R-CBA-MDA catheter 61 shown with the electrode 67 withdrawn from the catheter shaft central lumen and pressed against the internal wall of external carotid artery 6 adjacent to carotid body 59 using a force resulting from the pre-formed lateral expansion bias of arm 68 as shown. Indifferent RF electrode catheter 41 is shown residing in internal jugular vein 58. Indifferent electrode catheter 41 is depicted in FIG. 21 and described in detail below. R-CBA-MDA catheter 61 and indifferent electrode catheter are connected to an RF generator, not shown, ablation parameters are selected and ablation energy is applied to the wall of external carotid artery 6 resulting in ablation zone 107. Following application of ablation energy carotid body 59 function may then be evaluated. If carotid body 59 function is below a determined level, then R-CBA-MDA catheter 61 may be withdrawn. FIG. 15D is an in situ schematic illustration of the distal end of R-CBA-MDA catheter 61 shown with the electrode being positioned for carotid body 59 ablation from the wall of internal carotid artery 8 adjacent to carotid body 59 in the instance where carotid body function remained above the determined level following ablation from external carotid artery 6 described above. FIG. 15E is an in situ schematic illustration of the distal end of R-CBA-MDA catheter 61 showing the actuator clasp 70 and actuator clasp wire 71 pulled in the proximal direction to apply a pinching force to the carotid bifurcation 2. RF ablation energy is applied forming expanded ablation zone 107 comprising carotid body 59.

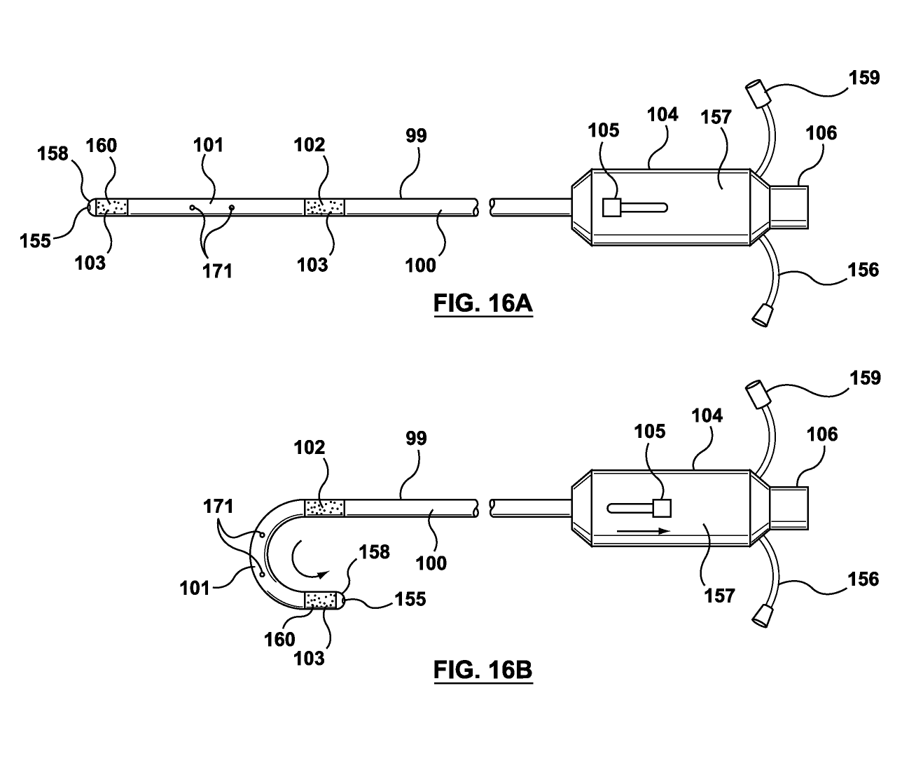

FIG. 16A is an illustration of a Retrograde Bipolar Carotid Body Ablation Deflectable J Tip (RB-CBA-DJT) catheter 99 configured for use through superficial temporal artery access comprising a tandem bipolar pair of RF electrodes 102 and 103 with a user actuated segment 101 between the electrode pair in its insertion configuration. FIG. 16B is an illustration of R-CBA-DJT catheter 99 in its actuated configuration. RB-CBA-DJT catheter 99 comprises catheter shaft 100, deflectable catheter segment 101, proximal electrode 102, and proximal handle assembly 104. Proximal handle assembly 104 comprises proximal handle 157, tip actuator 105, bipolar RF electrical connector 106, and fluid connector 156. Catheter shaft 100 is extruded from a polymeric material commonly used for vascular catheters, which may be a polyethylene, polyurethane, or nylon or other polymeric compound. Catheter shaft 100 may also have a woven, knitted, or coiled structure within its walls configured to translate rotational forces from its proximal end to its distal end to facilitated rotational positioning of ablation electrodes 102 and 103 and deflectable segment 101. Central lumen 155 is in fluidic communication between the distal end 158 of RB-CBA-DJT catheter 99 as shown and fluid connector 156, which may be a female luer connector at the proximal end. Central lumen 155 may be configured for use with a guide wire between approximately 0.014″ to 0.038″ in diameter. Central lumen 55 also provides a means for injecting a radiographic or ultrasonic contrast medium into the carotid vasculature for imaging assisted positioning of ablation electrodes 102 and 103 against the wall of an external carotid artery, and internal carotid artery respectively adjacent to a target site (e.g., carotid body, carotid body nerves, intercarotid septum). Optionally, central lumen 155 may be in fluid communication with one or more fluid exit ports 171 positioned along deflectable catheter segment 101 and an exit port at distal tip 158 may be absent. This may allow contrast medium to be injected from the fluid exit ports 171 into both an internal and external carotid artery to facilitate radiographic or ultrasonic imaging of the placement of electrodes 102 and 103 in relation to vessels, carotid bifurcation 2, or inter carotid septum 168. Optionally, catheter shaft 100 and deflectable catheter segment 101 may comprise a fluid lumen between irrigation fluid connector 159 and at least one fluid port(s) 160 in the vicinity of ablation electrodes 102 and 103 which may be used for irrigating the surface of electrodes 102 and 103 for the purpose cooling, or preventing thrombus formation on electrodes 102 and 103. Fluid port(s) 160 may comprise multiple micro-drilled holes in electrode surfaces 102 and 103, as shown, in communication with irrigation lumen, not shown, and irrigation fluid connector 159. Proximal electrode surface 50 is an exposed surface of proximal electrode ring 143. Deflectable segment 101 has a caliber approximately the same as catheter shaft 100, and has a length between ablation electrodes 102 and 103 of between approximately 1 cm and 4 cm (e.g., about 2 cm, between about 1.5 and 2.5 cm). Deflectable segment 101 is extruded from a polymeric material commonly used for vascular catheters, which may be a polyethylene, polyurethane, or nylon or other polymeric compound. Catheter shaft 100 may also comprise a coiled structure within its walls configured to translate compressive force from a pull wire, not shown, into a semi-circular deflection as shown without kinking or buckling. Deflectable segment 101 may be extruded from a softer polymer than that used in catheter shaft 100 so that catheter shaft 100 remains relatively straight when compressive force is applied by the pull wire. Those skilled in the art of deflectable tipped catheters are familiar with the design and construction of deflectable catheter segment utilizing a pull wire, therefore further description is not warranted. The pull wire, not shown is in mechanical communication between distal tip 158, and actuator 105. Ablation electrodes 102 and 103 are in electrical communication with electrical connector 106 by wires within deflectable segment 101 and catheter shaft 100. Electrical connector 106 is configured to connect ablation electrode 102 to one pole of an RF generator, not shown, and to connect ablation electrode 103 to the opposite pole of the RF generator. Ablation electrodes 102 and 103 are disposed on the surface of catheter shaft 100 and deflectable segment 101 as shown. Ablation electrodes 102 and 103 have an outer diameter approximately the same as catheter shaft 100 and deflectable segment 101, and have a wall thickness between approximately 0.002″ and 0.006″. Ablation electrodes 102 and 103 may be fabricated from an alloy with high thermal conductivity and high radiopacity such as a gold or platinum alloy. Handle 157 is ergonomically designed so the user may comfortably hold the handle, maneuver RB-CBA-DJT catheter 99 axially and rotationally, and maneuver actuator 105. As shown, actuator 105 is a sliding mechanism, where in the forward or distal position deflectable segment is substantially straight as shown in FIG. 16A, and in the backwards or proximal position deflectable segment 101 is substantially curved placing ablation electrodes 102 and 103 in lateral opposition to each other as shown in FIG. 16B. Fluid connectors 156 and 159 may be configured with a female luer fitting to facilitate connection to a syringe or other common fluid sources.

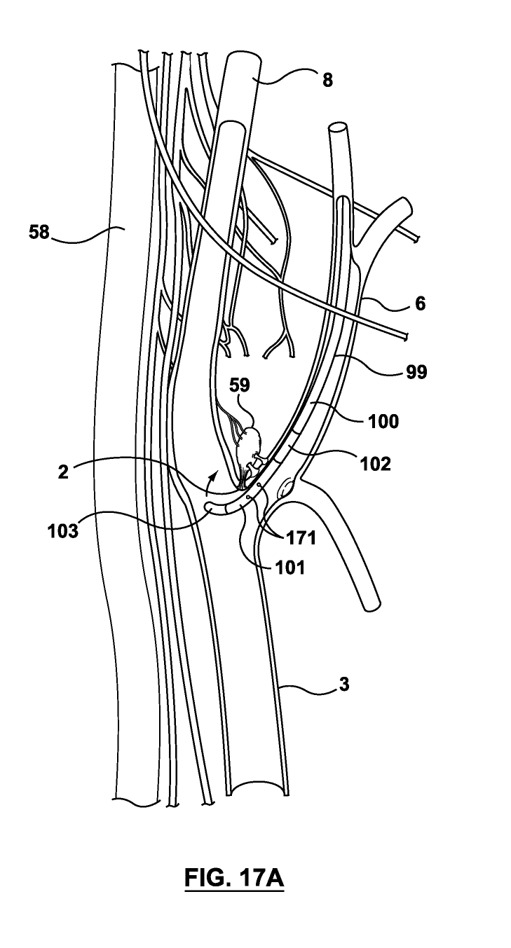

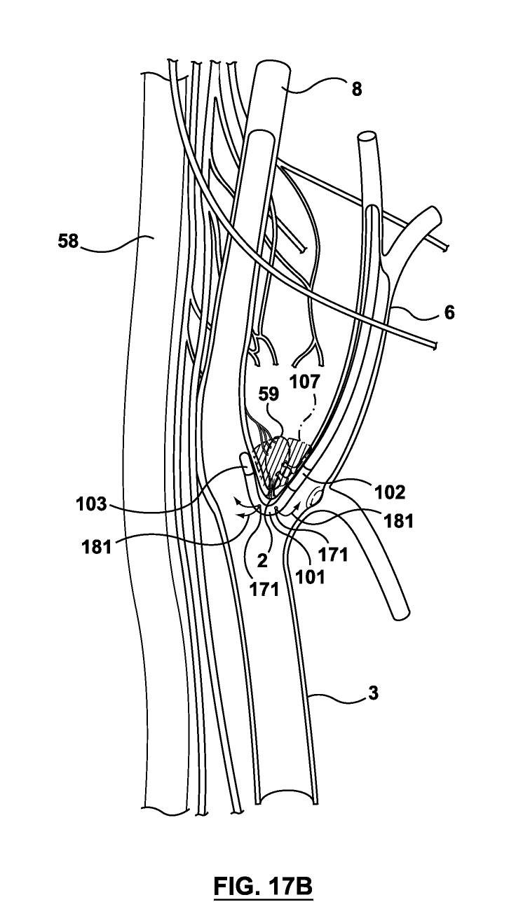

FIG. 17A is an in situ schematic illustration RB-CBA-DJT catheter 99 being positioned for use at the carotid bifurcation. FIG. 17B is an in situ schematic illustration of RB-CBA-DJT catheter 99 in position for carotid body modulation at the carotid bifurcation. RB-CBA-DJT catheter 99 is placed into the vicinity of carotid bifurcation 2 using fluoroscopic or ultrasonic by means of a trans-temporal artery introducer sheath, not shown, or by direct insertion to a superficial temporal artery using surgical cut down. Distal ablation electrode 103 is advanced into common carotid artery 3 with proximal ablation electrode 102 at approximately the level of a target site (e.g., carotid body 59). Contrast 181 may be deposited from central lumen 55 to blood flow, for example in common carotid artery 3 so it flows in to internal carotid artery 8 and external carotid artery 6 to facilitate imaging of carotid bifurcation 2 and relative positioning of electrodes. Using a combination of rotational positioning and actuation of deflectable segment 101, distal electrode 103 is pressed against the wall of internal carotid artery 8, adjacent to carotid body 59, and proximal electrode 102 is pressed against the wall of external carotid artery 6 adjacent to a target site (e.g., carotid body 59). The distance between proximal ablation electrode 102 and distal ablation electrode 103 is configured to place both electrodes an a suitable position for safe and effective carotid body modulation, for example, according to regions 174, 175, 176 and 177 shown in FIGS. 3A and 3B. Electrical connector 106 is connected to a RF generator, not shown. Ablation parameters are selected, and ablation energy is applied resulting in ablation zone 107 comprising carotid body 59.

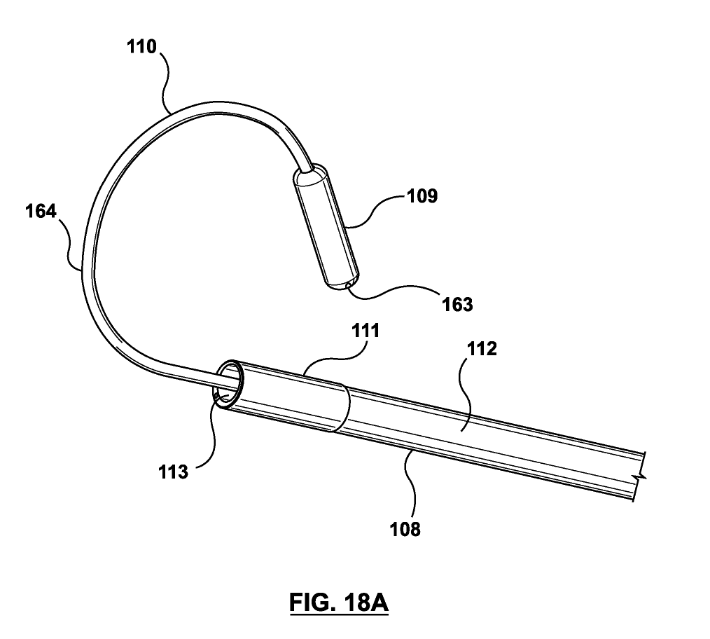

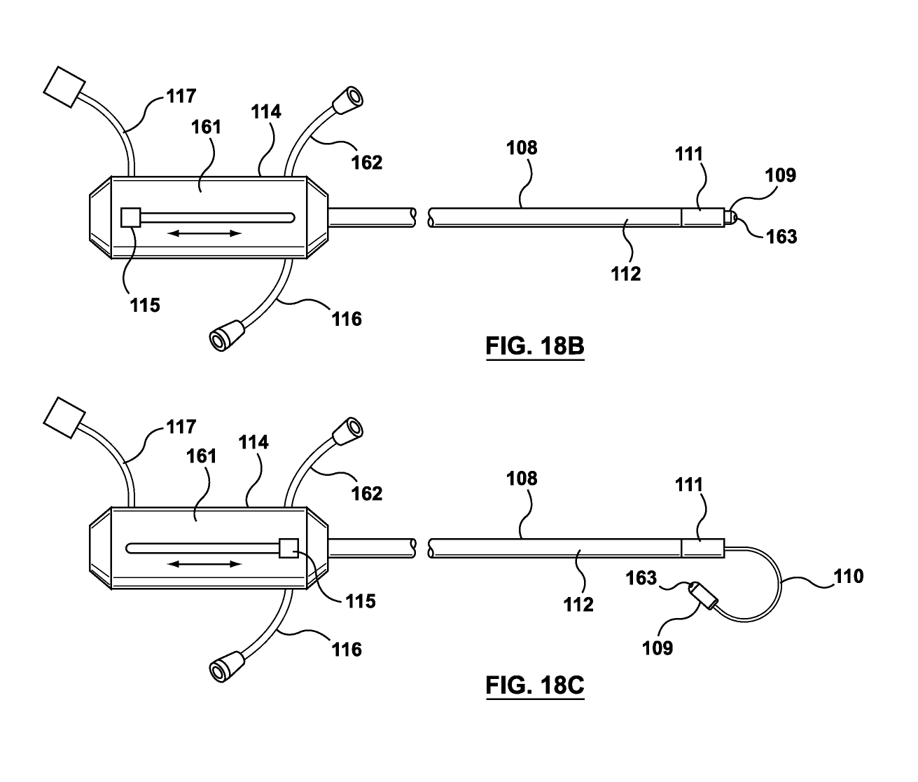

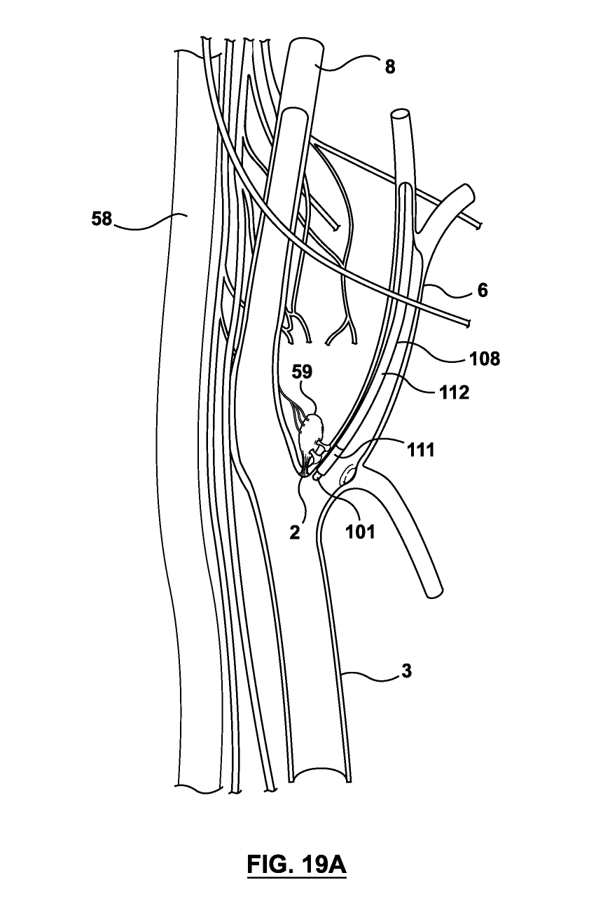

FIG. 18A is an illustration of the distal end of a Retrograde Bipolar Carotid Body Ablation Passive J Tipped (RB-CBA-PJT) catheter 108 comprising a telescopically deployable and retractable RF ablation electrode 109 mounted on an elastically deformable, preformed J-Wire 110, and a second RF ablation electrode 111 mounted on the surface at the distal end of the RB-CBA-PJT catheter 108. FIG. 18B is an illustration of RB-CBA-PJT catheter 108 in its insertion configuration. FIG. 18C is an illustration of RB-CBA-PJT catheter 108 in its use configuration. RB-CBA-PJT catheter 108 comprises: catheter shaft 112, ring electrode 111, J-Tip electrode 109, J-Wire 110, and handle assembly 114. Handle assembly 114 comprises handle 161, J-Tip actuator 115, central lumen fluid connector 116, bipolar RF electrical connector 117, and J-Wire fluid connector 162. Catheter shaft 112 has a caliber between approximately 3 French and 6 French, and a length between approximately 10 cm and 25 cm. Catheter shaft 112 is extruded from a polymeric material commonly used for vascular catheters, which may be a polyethylene, polyurethane, or nylon or other polymeric compound. Catheter shaft 112 may also have a woven, knitted, or coiled structure within its walls configured to translate rotational forces from its proximal end to its distal end to facilitate rotational positioning of J-Tip electrode 109. Central lumen 113 is in fluidic communication between the distal end of catheter shaft 112 and fluid connector 116, which may comprise a female luer connector at the proximal end of RB-CBA-PJT catheter 108. Central lumen 113 may be configured to house J-Tip electrode 109 during insertion and removal from the patient as depicted in FIG. 18B. Alternatively, central lumen 113 may be smaller and be configured to house J-Wire 110, in which case electrode 109 may protrude from the distal opening of central lumen 113 (not shown). Central lumen 113 also provides a means for injecting a radiographic or ultrasonic contrast medium into the carotid vasculature for imaging assisted positioning of ablation electrodes 111 and 109 against the wall of an external carotid artery, and internal carotid artery respectively adjacent to a target site. J-Wire 110 is between approximately 0.010 and 0.040″ in diameter, and may be a solid wire, or a hollow structure with a central lumen 163 as shown. J-Wire 110 may be fabricated from a super-elastic nickel-titanium alloy, which may have an electrically insulative coating such as a polymer heat shrink (e.g., PET) or vapor deposition coating (e.g., Parylene). Curved segment 164 is pre-formed in J-Wire 110 and may have a radius between approximately 5 mm and 10 mm, with an arch of between approximately 180 degrees to 330 degrees as shown, and a maximum length extending from the distal opening of central lumen 113 between about 1 cm and 4 cm (e.g., about 2 cm, between about 1.5 and 2.5 cm). The distance between proximal ablation electrode 111 and distal ablation electrode 109 is configured to place both electrodes an a suitable position for safe and effective carotid body modulation, for example, according to regions 174, 175, 176 and 177 shown in FIGS. 3A and 3B (e.g., the distance between electrodes along the length of the wire may be between about 4 and 20 mm, about 15 mm, about 12 mm, about 10 mm). J-Tip electrode 109 is mounted on the distal end of J-wire 110 by soldering, welding, swaging or other attachment means that provides for electrical connection between J-Tip electrode 109 and J-Wire 110. J-Tip electrode 109 is between approximately 1 mm and 1.5 mm in diameter, and is between approximately 3 mm and 10 mm in length. J-Tip electrode 109 may be fabricated from an alloy with high thermal conductivity and high radiopacity such as a gold or platinum alloy. J-wire 110 is connected to actuator 115 at its proximal end. J-Wire central lumen 163 is in fluidic communication between J-Wire fluid connector 162 at the proximal end and the open end of J-Wire central lumen 163 at the distal end of J-Tip electrode 109. J-wire central lumen 163 may be used to inject fluoroscopic contrast agent into the carotid vasculature to aid in fluoroscopic positioning of the distal end of RB-CBA-PJT catheter 108 for carotid body modulation. J-Wire 110 is coated with an electrically insulative material for substantially its entire length, which may comprise a polymeric coating such as PTFE, polyurethane, polyethylene, polyurethane, polyimide, or another polymeric material, or may be non-polymeric coating such as a ceramic coating. J-Wire 110 is in electrical communication with one contact of Bipolar RF connector 117. Ring electrode 111 is disposed on the surface of catheter shaft 112 in the vicinity of the distal end. Ring electrode 111 has an outer diameter approximately the same as catheter shaft 112, and has a wall thickness between approximately 0.002″ and 0.006″. Ring electrode 111 is connected to a second contact of bipolar RF connector 117 by an electrical wire residing within catheter shaft 112. Ring electrode 111 may be fabricated from an alloy with high thermal conductivity and high radiopacity such as a gold or platinum alloy. Handle 161 is ergonomically designed so the user may comfortably hold the handle, maneuver RB-CBA-PJT catheter 108 axially and rotationally, and manipulate J-Wire actuator 115. As shown, actuator 115 is a sliding mechanism, where in the forward or distal position J-Wire 110 may be extended as shown in FIG. 15C, and in the backwards or proximal position, J-Tip electrode 109 may be retracted into central lumen 113 as depicted in FIG. 15B. Fluid connectors 156 and 159 may be configured with a female luer fitting to facilitate connection to a syringe or other common fluid sources.

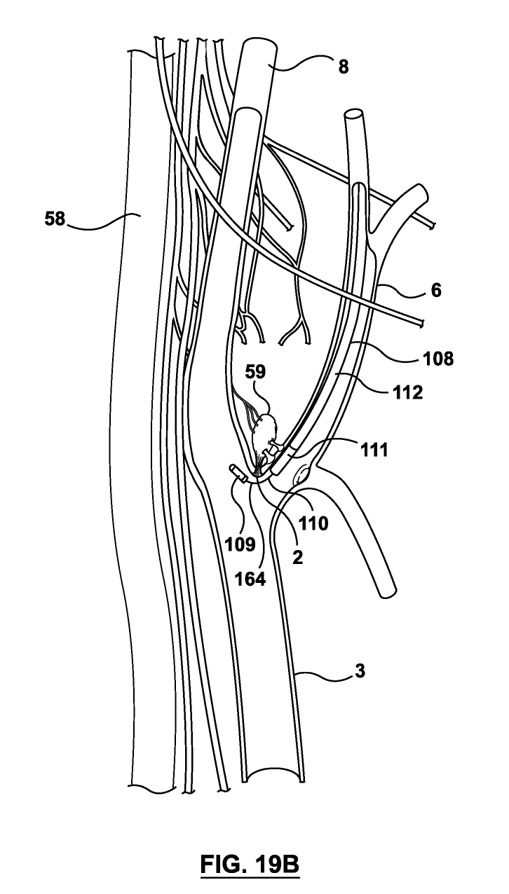

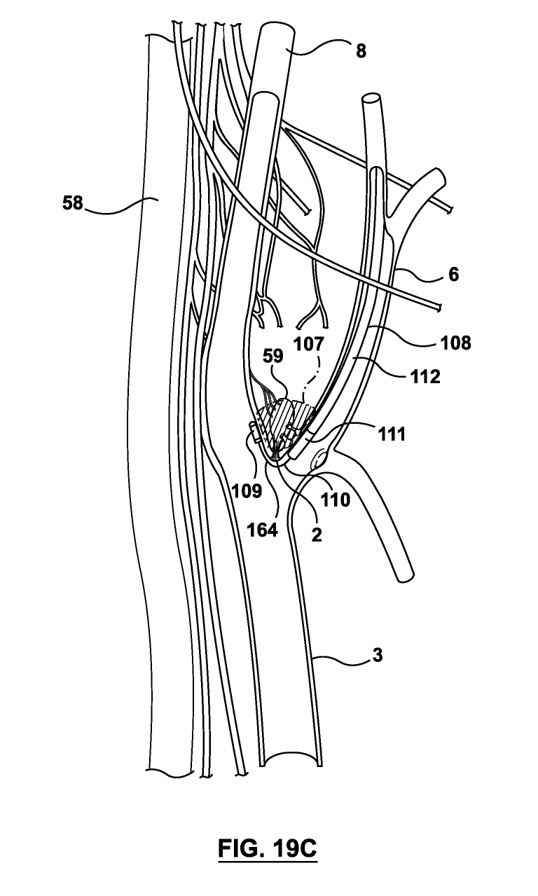

FIG. 19A is an in situ schematic illustration of RB-CBA-PJT catheter 108 being positioned for use at the carotid bifurcation 2 with ring electrode 111 within external carotid artery 6 at the approximate level of carotid body 59. RB-CBA-PJT catheter 108 is placed into the vicinity of carotid bifurcation 2 using fluoroscopic or ultrasonic imaging guidance by means of a trans-temporal artery introducer sheath, not shown, or by direct insertion to a superficial temporal artery using surgical cut down technique. FIG. 19B is an in situ schematic illustration of RB-CBA-PJT catheter 108 depicting J-Tip electrode 109 being extended and entering internal carotid artery 8 from external carotid artery 6. At this depicted position, radiographic contrast agent may be injected into internal carotid artery 8 through J-Wire central lumen 163 to provide the user with fluoroscopic information regarding the position of J-Tip electrode 109, and the morphology of internal carotid artery 8. FIG. 19C is an in situ schematic illustration of RB-CBA-PJT catheter 108 in position for carotid body 59 ablation. Using a combination of rotational positioning and actuation of J-Wire actuator 115, J-Wire electrode 109 is pressed against the wall of internal carotid artery 8, adjacent to carotid body 59, and ring electrode 111 is pressed against the wall of external carotid artery 6 adjacent to carotid body 59 using the spring effect of pre formed curved segment 164 of J-Wire 110. Bipolar RF electrical connector 117 is connected to an RF generator, not shown. Ablation parameters are selected, and ablation energy is applied resulting in ablation zone 107 comprising carotid body 59.

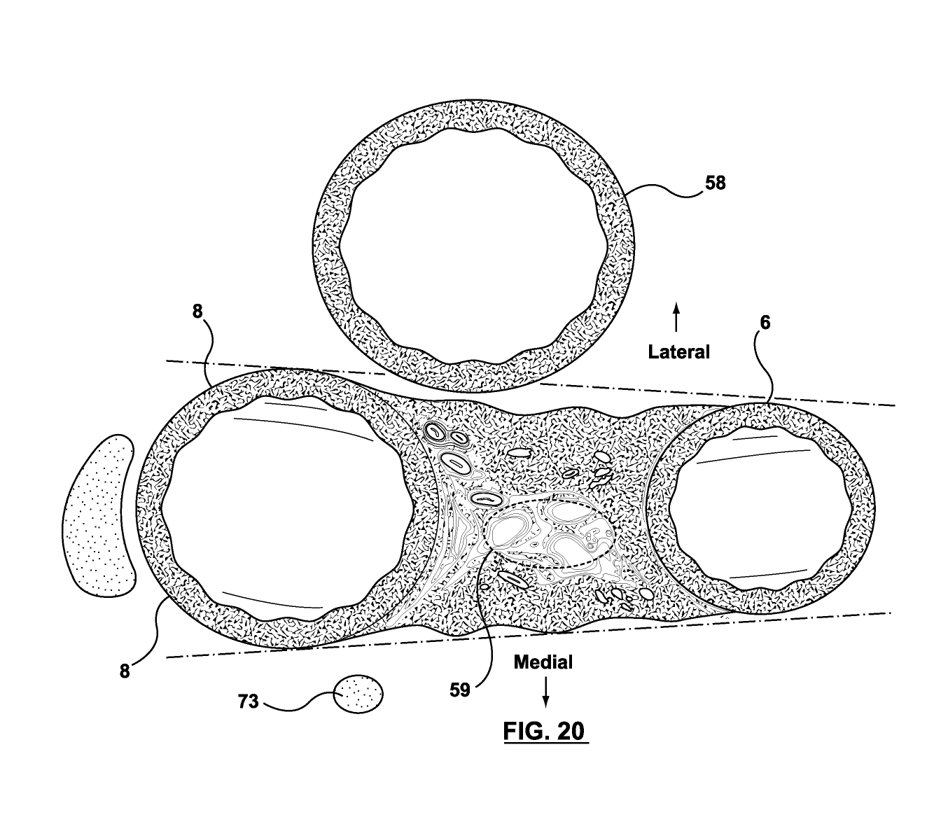

FIG. 20 is a transverse schematic illustration of the carotid arteries, external carotid artery 6 and internal carotid artery 8 immediately distal to the carotid bifurcation 2 showing the relative locations of the carotid body 59, internal jugular vein 58, and sympathetic nerve 73. Sympathetic nerve 73 is an important non-target nervous structure, located on the medial side of carotid bifurcation 2, to which thermal injury to sympathetic nerve 73 resulting from ablation of carotid body 59 function is clinically unacceptable.

FIG. 21 is an illustration of Jugular Indifferent Electrode (JIE) catheter 41 configured for use in a major lateral vein of the neck of a patient intended to prevent RF current from damaging important non-target nervous structures medial to carotid bifurcation during carotid body modulation. JIE catheter 41 comprises indifferent electrode 46, outer catheter shaft 44, inner catheter shaft 42, cage 165, central lumen 43, and proximal terminal, not shown. Proximal terminal comprises cage actuator, RF electrical connector, and a fluid connector, not shown. Cage 165 comprises proximal cage mounting ring 48, distal cage mounting ring 47, and cage struts 45. Outer catheter shaft 44 and inner catheter shaft 42 are in a slidable relationship. Indifferent electrode 46 is disposed on inner catheter shaft 42. Proximal cage mounting ring 48 is disposed on and fixated at the distal end of outer catheter shaft 44. Distal cage mounting ring 47 is disposed on and fixated near the distal end of inner catheter shaft 42 as shown. Cage 165 is configured for radial expansion of struts 45 in response to compressive forces that are generated when the distance between proximal gage mounting ring 48 and distal cage mounting ring 47 are reduced, and radial contraction of cage struts 45 when the distance between proximal cage ring 48 and distal cage mounting ring 47 is increased. The distance between proximal cage mounting ring 48 and distal cage mounting ring 47 is determined by the relative axial relationship between external catheter shaft 44, and internal catheter shaft 42. The axial relationship between outer catheter shaft 44 and internal catheter shaft 42 may be manipulated by an actuator mechanism located in the vicinity of the proximal terminal, not shown. Indifferent electrode 46 is connected to an RF electrical connector in the vicinity of the proximal terminal, not shown by an electrical conductor within internal catheter shaft 42. Indifferent electrode 46 is between approximately 1 mm and 3 mm in diameter, and has a length between approximately 10 mm and 25 mm. Indifferent electrode 46 has a wall thickness between 0.1 mm and 0.3 mm, and may be fabricated from a metallic alloy with high thermal conductivity and radiopacity such as a gold or platinum alloy. Central lumen 43 is in fluidic communication with a fluid connector in the vicinity of the proximal end, not show, and may be configured for injection of a radiographic or ultrasonic contrast agent, and may further be configured for use with a guidewire. Outer catheter shaft 44, and inner catheter shaft 42, are extruded from a polymeric compound, which may be a polyethylene, polyurethane, nylon, or other catheter material. Outer catheter shaft 44 or inner catheter shaft 42 may comprise a woven, knitted, or coiled structure to provide torsional, or axial rigidity. Cage 165 is configured to prevent indifferent electrode 46 from touching a vascular wall, thereby preventing thermal injury to the vascular wall during application of RF energy, and to allow blood to flow over the surface of indifferent electrode 46 to provide convective cooling of indifferent electrode 46.

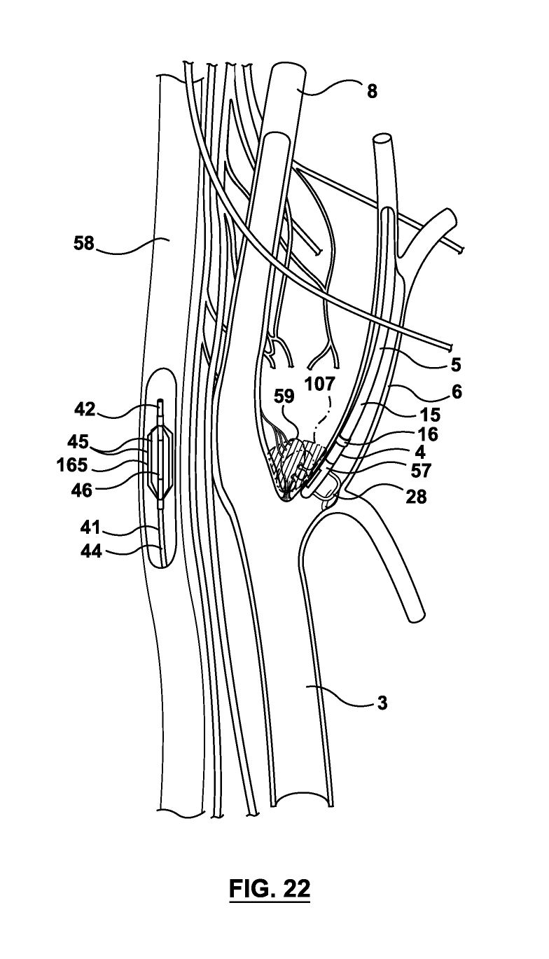

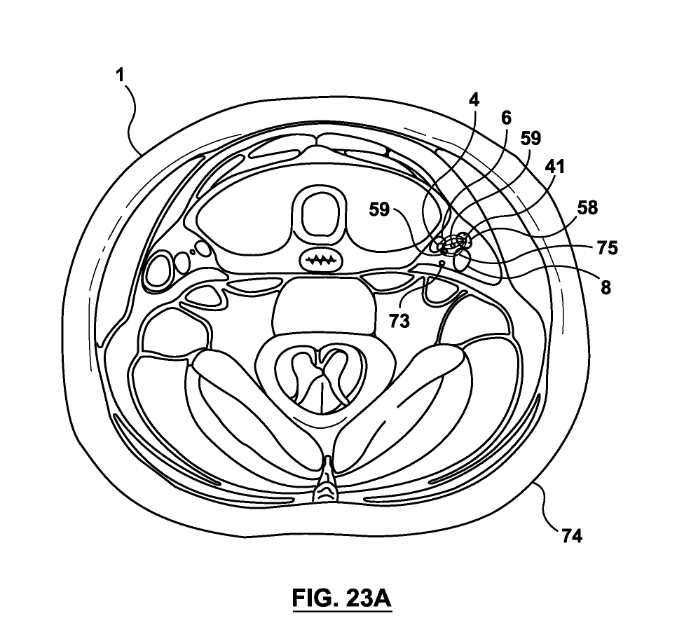

FIG. 22 is a schematic illustration of a monopolar RF carotid body modulation catheter 4 in situ utilizing access to the region of the carotid body 59 from a superficial temporal artery puncture and a JIE catheter 41 located in the associated internal jugular vein. Ablation element 57 is shown being pressed against the wall of external carotid artery 6 by push wire 28. Cage 165 is depicted in its expanded position within internal jugular vein 58 with struts 45 engaging the wall of internal jugular vein 58 at the approximate level of carotid body 59. Carotid body modulation is represented by ablation zone 107. Location of indifferent electrode 46 lateral to carotid body 59 within internal jugular vein 58 directs RF current between indifferent electrode 46 and ablation element electrode 57 diminishing the density of RF current medial to carotid body 59, thereby diminishing the risk of thermal injury to important non-target medial nervous structures, such as the sympathetic nerve 73.

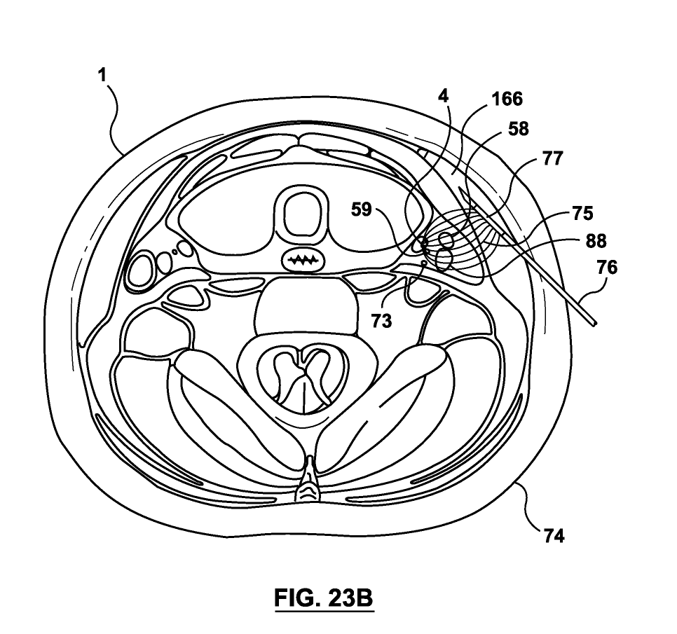

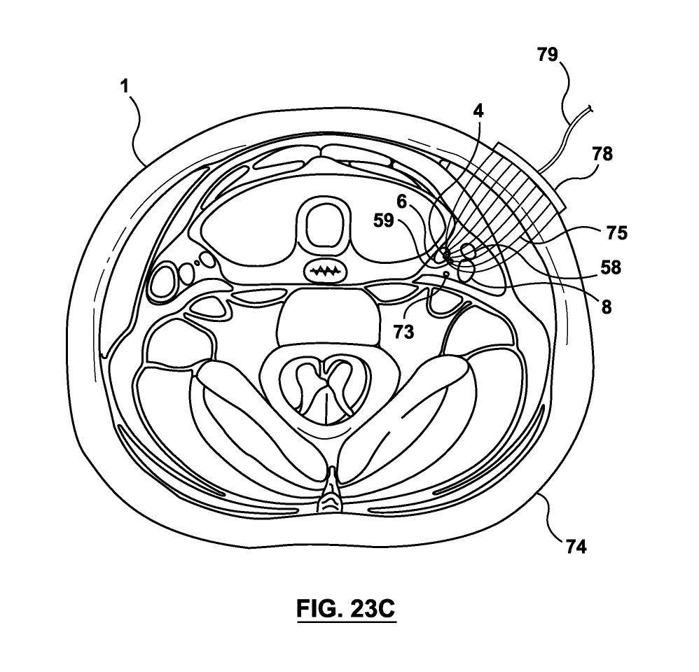

FIG. 23A is a transverse sectional schematic illustration of a patient's neck 1 depicting a monopolar RF ablation catheter 4 residing within the external carotid artery 8 in position for carotid body 59 ablation, with an JIE catheter 41 residing within the internal jugular vein 58, showing the RF current path 75 between the RF catheter's 4 ablation electrode, and the indifferent electrode on the JIE catheter 41. FIG. 23B is a transverse sectional schematic illustration of a patient's neck 1 depicting a monopolar RF ablation catheter 4 residing within the external carotid 6 artery in position for carotid body 59 ablation, and a percutaneous indifferent electrode probe 76 inserted into neck muscle 166 adjacent to carotid body 59, showing the RF current path 75 between the RF catheter 4 ablation electrode, and the indifferent electrode 77 on the indifferent electrode probe 76. FIG. 23C is a transverse sectional schematic illustration of a patient's neck 1 depicting a monopolar RF ablation catheter 4 residing within the external carotid artery 6 in position for carotid body 59 ablation, and an indifferent electrode skin pad 78 on the patient's neck adjacent to carotid body 59, showing the RF current path 75 between the RF catheter 4 ablation electrode, and the indifferent electrode skin pad 78.

### System

A system has been conceived comprising a catheter configured to access a target site via a superficial temporal artery for carotid body modulation, and an ablation energy console. The system may additionally comprise a connector cable or several cables for connecting the ablation energy console with the catheter, a delivery sheath, or a guide wire. The console may be configured to deliver ablation energy to the catheter. For example, the console may be an electrical signal generator such as a radiofrequency generator or an irreversible electroporation generator. The console may further comprise a user interface that provides the user with a means to select ablation parameters, activate and deactivate an ablation, or to monitor progress of an ablation. The console may have a second user interface that allows the user to select electrical stimulation or blockade used to investigate proximity of an ablation element on the catheter to neural structures. The console may comprise a computer algorithm that controls ablation energy delivery. The algorithm may control energy delivery (e.g., controlled power delivery) based on inputs for example, user selected variables, intercarotid septum width, pre-programmed variables, physiologic signals (e.g., impedance, temperature), or sensor feedback. Selectable carotid body modulation parameters may include ablation element temperature, duration of ablation element activation, ablation power, ablation element force of contact with a vessel wall, ablation element size, ablation modality, and ablation element position within a vessel.

Pressure or force sensors may be incorporated into any of the catheter embodiments described above, for example they could be mounted to a flex circuit proximate an ablation element, and could be used to verify contact or indicate contact force. Diverging arms with open/close actuation or deployable structures used to obtain electrode contact with a vessel wall could be actuated to a position that corresponds to a particular contact pressure range. Alternatively, a catheter could be “pushed” against the wall until contact pressure reaches a desired level. Alternatively, a baseline pressure may be chosen when a desirable contact force is visually confirmed, for example vessel distension caused by ablation element contact force may visually appear using an imaging modality such as angiography. A change of pressure or force, within an acceptable range from the baseline, measured by the sensors may indicate appropriate contact force and deviation from this range could indicate an inappropriate contact force. A computer algorithm that controls delivery of ablation energy may discontinue energy delivery if contact force deviates from the appropriate range. Furthermore, a pressure sensor may be used to indicate absolute or relative blood flow and power delivery could be augmented by feedback from the pressure sensor. Alternatively, a temperature sensor, cooled by blood flow, can be used to determine blood flow velocity. Blood flow cooling can be factored into the control algorithms as correction of energy delivery. Also sudden drop of blood flow can indicate spasm of the carotid vessel. Such an abrupt temperature rise will indicate a need to stop or reduce energy delivery instantly. For example, low flow may equal less power and/or power delivery duration, while greater flow may result in more power and/or longer duration. Power of ablation energy delivery may be decreased or duration of energy delivery may be reduced if the flow decreases. Conversely, should the flow increase power or duration may be increased. Alternatively, a pressure sensor may be used to track potential damage to nerves that are to be preserved. Heart rate may be inferred from a pressure sensor through pulsatile flow. The right vagus nerve primarily innervates the sinoatrial node while the left vagus nerve primarily innervates the atrioventricular node. Should either vagus nerve become stimulated, blocked or damaged the patient's heart rate may fluctuate or decline, which may be indicated by the pressure or flow sensor an energy delivery algorithm may stop power delivery or provide a warning accordingly. Similarly, heart function and some gauge of instantaneous heart rate variability may be measured in other ways (e.g., ECG, plethysmography, pulse oximetry) and used by an energy delivery algorithm for safety.

Tissue impedance, phase or capacitance may be measured between two electrodes in a bipolar arrangement, or between an electrode and a dispersive electrode in a monopolar arrangement. Impedance measurement across an intercarotid septum may be used to indicate distance between electrodes, position on a bifurcation, tissue characteristics, ablation characteristics, electrode contact with tissue, or catheter integrity. An energy delivery algorithm may incorporate impedance feedback, phase changes, or temperature to control delivery of ablation energy. For example, these feedback variables may be used to modulate energy delivery or as a safety cut-off. Ablation energy may be delivered for a predetermined duration of time (e.g., between about 20 and 90 s, or in a range of about 20-30 s) and energy delivery may be reduced or stopped if there is indication that a traumatic event or a poor ablation is about to happen, such as high temperature or temperature above set point, which may lead to events such as charring or coagulation, or significant movement or poor contact of the electrodes with respect to tissue, which may lead to unpredictable ablation or ablation at a non-target region. A bipolar arrangement may be more sensitive to impedance changes and be able to prepare the generator to shut off more quickly than a monopolar arrangement. For example, a bipolar radiofrequency configuration may provide an improved signal to noise ration compared to a monopolar configuration and may provide a clear indication that electrodes are moving. However, an energy delivery control algorithm for either a bipolar or monopolar configuration may incorporate feedback variables for ablation and safety control as discussed herein. For example, prior to charring, which may be indicated by a sharp spike in impedance, several cycles of impedance fluctuation may be measured; if electrode contact with tissue is compromised or electrode position has moved an acute impedance change and simultaneous temperature change at one or both electrodes may be measured; if a catheter is compromised a feedback signal from a temperature sensor may be severed or out of a reasonable range; if a vessel is undergoing spasm impedance and temperature fluctuations as well as power phase changes may be detected simultaneously and in a sinusoidal pattern or may be determined based on hysteresis. Any of these indications may result in a reduction of energy delivery power, power shut off, or a safety warning. Variables such as impedance and temperature may be an indication of a successful ablation. For example, changes in impedance (e.g., value and phase) may be measured when carotid body perfusion is coagulated. This may be an indication that target temperature is exceeding 50-60 C, which may be an indication of technical success. Energy delivery may be stopped or continued for a short amount of time after this occurs to limit a chance that a lesion grows into that hazards medial zone. Another way an energy delivery algorithm may incorporate impedance feedback, phase changes, or temperature to control delivery of ablation energy is to adjust power delivery to meet a set point temperature, impedance, phase or capacitance.

An ablation energy source (e.g., energy field generator) may be located external to the patient. Various types of ablation energy generators or supplies, such as electrical frequency generators, ultrasonic generators, microwave generators, laser consoles, and heating or cryogenic fluid supplies, may be used to provide energy to the ablation element at the distal tip of the catheter. An electrode or other energy applicator at the distal tip of the catheter should conform to the type of energy generator coupled to the catheter. The generator may include computer controls to automatically or manually adjust frequency and strength of the energy applied to the catheter, timing and period during which energy is applied, and safety limits to the application of energy. It should be understood that embodiments of energy delivery electrodes described hereinafter may be electrically connected to the generator even though the generator is not explicitly shown or described with each embodiment.

An ablated tissue lesion at or near the carotid body may be created by the application of ablation energy from an ablation element in a vicinity of a distal end of the carotid body modulation device. The ablated tissue lesion may disable the carotid body or may suppress the activity of the carotid body or interrupt conduction of afferent nerve signals from a carotid body to sympathetic nervous system. The disabling or suppression of the carotid body reduces the responsiveness of the glomus cells to changes of blood gas composition and effectively reduces activity of afferent carotid body nerves or the chemoreflex gain of the patient.

### Methods of Treatment

A method in accordance with a particular embodiment includes ablating at least one of a patient's carotid bodies based at least in part on identifying the patient as having a sympathetically mediated disease such as cardiac, metabolic, or pulmonary disease such as hypertension, insulin resistance, diabetes, pulmonary hypertension, drug resistant hypertension (e.g., refractory hypertension), congestive heart failure (CHF), or dyspnea from heart failure or pulmonary disease causes.

A procedure may include diagnosis, selection based on diagnosis, further screening (e.g., baseline assessment of chemosensitivity), treating a patient based at least in part on diagnosis or further screening via a chemoreceptor (e.g., carotid body) ablation procedure such as one of the embodiments disclosed. Additionally, following ablation a method of therapy may involve conducting a post-ablation assessment to compare with the baseline assessment and making decisions based on the assessment (e.g., adjustment of drug therapy, re-treat in new position or with different parameters, or ablate a second chemoreceptor if only one was previously ablated).

A carotid body modulation procedure may comprise the following steps or a combination thereof: patient sedation, locating a target peripheral chemoreceptor, visualizing a target peripheral chemoreceptor (e.g., carotid body), confirming a target ablation site is or is proximate a peripheral chemoreceptor, confirming a target ablation site is safely distant from important non-target nerve structures that are preferably protected (e.g., hypoglossal, sympathetic and vagus nerves), providing stimulation (e.g., electrical, mechanical, chemical) to a target site or target peripheral chemoreceptor prior to, during or following an ablation step, monitoring physiological responses to said stimulation, providing temporary nerve block to a target site prior to an ablation step, monitoring physiological responses to said temporary nerve block, anesthetizing a target site, protecting the brain from potential embolism, thermally protecting an arterial or venous wall (e.g., carotid artery, jugular vein) or a medial aspect of an intercarotid septum or non-target nerve structures, ablating a target site (e.g., peripheral chemoreceptor), monitoring ablation parameters (e.g., temperature, pressure, duration, blood flow in a carotid artery), monitoring physiological responses during ablation and arresting ablation if unsafe or unwanted physiological responses occur before collateral nerve injury becomes permanent, confirming a reduction of chemoreceptor activity (e.g., chemosensitivity, HR, blood pressure, ventilation, sympathetic nerve activity) during or following an ablation step, removing a ablation device, conducting a post-ablation assessment, repeating any steps of the chemoreceptor ablation procedure on another peripheral chemoreceptor in the patient.

The location of the perivascular space associated with a carotid body may be determined by means of a non-fluoroscopic imaging procedure prior to carotid body modulation, where the non-fluoroscopic location information is translated to a coordinate system based on fluoroscopically identifiable anatomical and/or artificial landmarks.

A function of a carotid body may be stimulated and at least one physiological parameter is recorded prior to and during the stimulation, then the carotid body is ablated, and the stimulation is repeated, whereby the change in recorded physiological parameter(s) prior to and after ablation is an indication of the effectiveness of the ablation, and the resultant degree of carotid body modulation.

A function of a carotid body may be temporarily blocked and at least one physiological parameter(s) is recorded prior to and during the blockade, then the carotid body is ablated, and the blockade is repeated, whereby the change in recorded physiological parameter(s) prior to and after ablation is an indication of the effectiveness of the ablation.

Patient screening, as well as post-ablation assessment may include physiological tests or gathering of information, for example, chemoreflex sensitivity, central sympathetic nerve activity, heart rate, heart rate variability, blood pressure, ventilation, production of hormones, peripheral vascular resistance, blood pH, blood PCO2, degree of hyperventilation, peak VO2, VE/VCO2 slope. Directly measured maximum oxygen uptake (more correctly pVO2 in heart failure patients) and index of respiratory efficiency VE/VCO2 slope has been shown to be a reproducible marker of exercise tolerance in heart failure and provide objective and additional information regarding a patient's clinical status and prognosis.

A method of therapy may include electrical stimulation of a target region, using a stimulation electrode, to confirm proximity to a carotid body. For example, a stimulation signal having a 1-10 milliamps (mA) pulse train at about 20 to 40 Hz with a pulse duration of 50 to 500 microseconds (μs) that produces a positive carotid body stimulation effect may indicate that the stimulation electrode is within sufficient proximity to the carotid body or nerves of the carotid body to effectively ablate it. A positive carotid body stimulation effect could be increased blood pressure, heart rate, or ventilation concomitant with application of the stimulation. These variables could be monitored, recorded, or displayed to help assess confirmation of proximity to a carotid body. A catheter-based technique, for example, may have a stimulation electrode proximal to the ablation element used for ablation. Alternatively, the ablation element itself may also be used as a stimulation electrode. Alternatively, an energy delivery element that delivers a form of ablative energy that is not electrical, such as a cryogenic ablation applicator, may be configured to also deliver an electrical stimulation signal as described earlier. Yet another alternative embodiment comprises a stimulation electrode that is distinct from an ablation element. For example, during a surgical procedure a stimulation probe can be touched to a suspected carotid body that is surgically exposed. A positive carotid body stimulation effect could confirm that the suspected structure is a carotid body and ablation can commence. Physiological monitors (e.g., heart rate monitor, blood pressure monitor, blood flow monitor, MSNA monitor) may communicate with a computerized stimulation generator, which may also be an ablation generator, to provide feedback information in response to stimulation. If a physiological response correlates to a given stimulation the computerized generator may provide an indication of a positive confirmation.

Alternatively or in addition a drug known to excite the chemo sensitive cells of the carotid body can be injected directly into the carotid artery or given systemically into a patient's vein or artery in order to elicit hemodynamic or respiratory response. Examples of drugs that may excite a chemoreceptor include nicotine, atropine, Doxapram, Almitrine, hyperkalemia, Theophylline, adenosine, sulfides, Lobeline, Acetylcholine, ammonium chloride, methylamine, potassium chloride, anabasine, coniine, cytosine, acetaldehyde, acetyl ester and the ethyl ether of i-methylcholine, Succinylcholine, Piperidine, monophenol ester of homo-iso-muscarine and acetylsalicylamides, alkaloids of veratrum, sodium citrate, adenosinetriphosphate, dinitrophenol, caffeine, theobromine, ethyl alcohol, ether, chloroform, phenyldiguanide, sparteine, coramine (nikethamide), metrazol (pentylenetetrazol), iodomethy late of dimethylaminomethylenedioxypropane, ethyltrimethylammoniumpropane, trimethylammonium, hydroxytryptamine, papaverine, neostigmine, acidity.

A method of therapy may further comprise applying electrical or chemical stimulation to the target area or systemically following ablation to confirm a successful ablation. Heart rate, blood pressure or ventilation may be monitored for change or compared to the reaction to stimulation prior to ablation to assess if the targeted carotid body was ablated. Post-ablation stimulation may be done with the same apparatus used to conduct the pre-ablation stimulation. Physiological monitors (e.g., heart rate monitor, blood pressure monitor, blood flow monitor, MSNA monitor) may communicate with a computerized stimulation generator, which may also be an ablation generator, to provide feedback information in response to stimulation. If a physiological response correlated to a given stimulation is reduced following an ablation compared to a physiological response prior to the ablation, the computerized generator may provide an indication ablation efficacy or possible procedural suggestions such as repeating an ablation, adjusting ablation parameters, changing position, ablating another carotid body or chemosensor, or concluding the procedure.

The devices described herein may also be used to temporarily stun or block nerve conduction via electrical neural blockade. A temporary nerve block may be used to confirm position of an ablation element prior to ablation. For example, a temporary nerve block may block nerves associated with a carotid body, which may result in a physiological effect to confirm the position may be effective for ablation. Furthermore, a temporary nerve block may block important non-target nerves such as vagal, hypoglossal or sympathetic nerves that are preferably avoided, resulting in a physiological effect (e.g., physiological effects may be noted by observing the patient's eyes, tongue, throat or facial muscles or by monitoring patient's heart rate and respiration). This may alert a user that the position is not in a safe location. Likewise absence of a physiological effect indicating a temporary nerve block of such important non-target nerves in combination with a physiological effect indicating a temporary nerve block of carotid body nerves may indicate that the position is in a safe and effective location for carotid body modulation.

Important nerves may be located in proximity of the target site and may be inadvertently and unintentionally injured. Neural stimulation or blockade can help identify that these nerves are in the ablation zone before the irreversible ablation occurs. These nerves may include the following:

Vagus Nerve Bundle—The vagus is a bundle of nerves that carry separate functions, for example a) branchial motor neurons (efferent special visceral) which are responsible for swallowing and phonation and are distributed to pharyngeal branches, superior and inferior laryngeal nerves; b) visceral motor (efferent general visceral) which are responsible for involuntary muscle and gland control and are distributed to cardiac, pulmonary, esophageal, gastric, celiac plexuses, and muscles, and glands of the digestive tract; c) visceral sensory (afferent general visceral) which are responsible for visceral sensibility and are distributed to cervical, thoracic, abdominal fibers, and carotid and aortic bodies; d) visceral sensory (afferent special visceral) which are responsible for taste and are distributed to epiglottis and taste buds; e) general sensory (afferent general somatic) which are responsible for cutaneous sensibility and are distributed to auricular branch to external ear, meatus, and tympanic membrane. Dysfunction of the vagus may be detected by a) vocal changes caused by nerve damage (damage to the vagus nerve can result in trouble with moving the tongue while speaking, or hoarseness of the voice if the branch leading to the larynx is damaged); b) dysphagia due to nerve damage (the vagus nerve controls many muscles in the palate and tongue which, if damaged, can cause difficulty with swallowing); c) changes in gag reflex (the gag reflex is controlled by the vagus nerve and damage may cause this reflex to be lost, which can increase the risk of choking on saliva or food); d) hearing loss due to nerve damage (hearing loss may result from damage to the branch of the vagus nerve that innervates the concha of the ear): e) cardiovascular problems due to nerve damage (damage to the vagus nerve can cause cardiovascular side effects including irregular heartbeat and arrhythmia); or f) digestive problems due to nerve damage (damage to the vagus nerve may cause problems with contractions of the stomach and intestines, which can lead to constipation).

Superior Laryngeal Nerve—the superior laryngeal nerve is a branch of the vagus nerve bundle. Functionally, the superior laryngeal nerve function can be divided into sensory and motor components. The sensory function provides a variety of afferent signals from the supraglottic larynx. Motor function involves motor supply to the ipsilateral cricothyroid muscle. Contraction of the cricothyroid muscle tilts the cricoid lamina backward at the cricothyroid joint causing lengthening, tensing and adduction of vocal folds causing an increase in the pitch of the voice generated. Dysfunction of the superior laryngeal nerve may change the pitch of the voice and causes an inability to make explosive sounds. A bilateral palsy presents as a tiring and hoarse voice.

Cervical Sympathetic Nerve—The cervical sympathetic nerve provides efferent fibers to the internal carotid nerve, external carotid nerve, and superior cervical cardiac nerve. It provides sympathetic innervation of the head, neck and heart. Organs that are innervated by the sympathetic nerves include eyes, lacrimal gland and salivary glands. Dysfunction of the cervical sympathetic nerve includes Homer's syndrome, which is very identifiable and may include the following reactions: a) partial ptosis (drooping of the upper eyelid from loss of sympathetic innervation to the superior tarsal muscle, also known as Müller's muscle); b) upside-down ptosis (slight elevation of the lower lid); c) anhidrosis (decreased sweating on the affected side of the face); d) miosis (small pupils, for example small relative to what would be expected by the amount of light the pupil receives or constriction of the pupil to a diameter of less than two millimeters, or asymmetric, one-sided constriction of pupils); e) enophthalmos (an impression that an eye is sunken in); loss of ciliospinal reflex (the ciliospinal reflex, or pupillary-skin reflex, consists of dilation of the ipsilateral pupil in response to pain applied to the neck, face, and upper trunk. If the right side of the neck is subjected to a painful stimulus, the right pupil dilates about 1-2 mm from baseline. This reflex is absent in Homer's syndrome and lesions involving the cervical sympathetic fibers.)

### Visualization:

An optional step of visualizing internal structures (e.g., carotid body, aortic arch, carotid arteries, or surrounding structures) may be accomplished using one or more non-invasive imaging modalities, for example fluoroscopy, radiography, arteriography, computer tomography (CT), computer tomography angiography with contrast (CTA), magnetic resonance imaging (MRI), or sonography, or minimally invasive techniques (e.g., IVUS, endoscopy, optical coherence tomography, ICE). A visualization step may be performed as part of a patient assessment, prior to an ablation procedure to assess risks and location of anatomical structures, during an ablation procedure to help guide an ablation device, or following an ablation procedure to assess outcome (e.g., efficacy of the ablation). Visualization may be used to: (a) locate a carotid body, (b) locate important non-target nerve structures that may be adversely affected, or (c) locate, identify and measure arterial plaque. Visualization may be used to assess an endovascular access path (e.g., retrograde, trans superficial temporal artery access, femoral access, radial access, brachial access) based, for example, on vessel structure, tortuosity, presence of plaque, or other limitations. A suitable carotid body modulation device may be chosen based on the most suitable access path. For example, a catheter configured for CBM via trans-superficial temporal artery access, such as the embodiments described herein, may be chosen for a patient having a vessel structure or other limitation that makes a femoral artery access procedure difficult or risky.

Endovascular (for example transfemoral) arteriography of the common carotid and then selective arteriography of the internal and external carotids may be used to determine a position of a catheter tip at a carotid bifurcation. Additionally, ostia of glomic arteries (these arteries may be up to 4 mm long and arise directly from the main parent artery) can be identified by dragging the dye injection catheter and releasing small amounts (“puffs”) of dye. If a glomic artery is identified it can be cannulated by a guide wire and possibly further cannulated by small caliber catheter. Direct injection of dye into glomic arteries can further assist the interventionalist in the ablation procedure. It is appreciated that the feeding glomic arteries are small and microcatheters may be needed to cannulate them.

Alternatively, ultrasound visualization may allow a physician to see the carotid arteries and even the carotid body. Another method for visualization may consist of inserting a small needle (e.g., 22 Gauge) with sonography or computer tomography (CT) guidance into or toward the carotid body. A wire or needle can be left in place as a fiducial guide, or contrast can be injected into the carotid body. Runoff of contrast to the jugular vein may confirm that the target is achieved.

Computer Tomography (CT) and computer tomography angiography (CTA) may also be used to aid in identifying a carotid body. Such imaging could be used to help guide an ablation device to a carotid body.

Ultrasound visualization (e.g., sonography) is an ultrasound-based imaging technique used for visualizing subcutaneous body structures including blood vessels and surrounding tissues. Doppler ultrasound uses reflected ultrasound waves to identify and display blood flow through a vessel. Operators typically use a hand-held transducer/transceiver placed directly on a patient's skin and aimed inward directing ultrasound waves through the patient's tissue. Ultrasound may be used to visualize a patient's carotid body to help guide an ablation device. Ultrasound can be also used to identify atherosclerotic plaque in the carotid arteries and avoid disturbing and dislodging such plaque.

Visualization and navigation steps may comprise multiple imaging modalities (e.g., CT, fluoroscopy, ultrasound) superimposed digitally to use as a map for instrument positioning. Superimposing borders of great vessels such as carotid arteries can be done to combine images.

Responses to stimulation at different coordinate points can be stored digitally as a 3-dimensional or 2-dimensional orthogonal plane map. Such an electric map of the carotid bifurcation showing points, or point coordinates that are electrically excitable such as baroreceptors, baroreceptor nerves, chemoreceptors and chemoreceptor nerves can be superimposed with an image (e.g., CT, fluoroscopy, ultrasound) of vessels. This can be used to guide the procedure, and identify target areas and areas to avoid.

In addition, as noted above, it should be understood that a device providing therapy can also be used to locate a carotid body as well as to provide various stimuli (electrical, chemical, other) to test a baseline response of the carotid body chemoreflex (CBC) or carotid sinus baroreflex (CSB) and measure changes in these responses after therapy or a need for additional therapy to achieve the desired physiological and clinical effects.

### Patient Selection and Assessment:

In an embodiment, a procedure may comprise assessing a patient to be a plausible candidate for carotid body modulation. Such assessment may involve diagnosing a patient with a sympathetically mediated disease (e.g., MSNA microneurography, measure of cataclomines in blood or urine, heart rate, or low/high frequency analysis of heart rate variability may be used to assess sympathetic tone). Patient assessment may further comprise other patient selection criteria, for example indices of high carotid body activity (i.e., carotid body hypersensitivity or hyperactivity) such as a combination of hyperventilation and hypocarbia at rest, high carotid body nerve activity (e.g., measured directly), incidence of periodic breathing, dyspnea, central sleep apnea elevated brain natriuretic peptide, low exercise capacity, having cardiac resynchronization therapy, atrial fibrillation, ejection fraction of the left ventricle, using beta blockers or ACE inhibitors.

Patient assessment may further involve selecting patients with high peripheral chemosensitivity (e.g., a respiratory response to hypoxia normalized to the desaturation of oxygen greater than or equal to about 0.7 l/min/min SpO₂), which may involve characterizing a patient's chemoreceptor sensitivity, reaction to temporarily blocking carotid body chemoreflex, or a combination thereof.

Although there are many ways to measure chemosensitivity they can be divided into (a) active provoked response and (b) passive monitoring. Active tests can be done by inducing intermittent hypoxia (such as by taking breaths of nitrogen or CO₂ or combination of gases) or by rebreathing air into and from a 4 to 10 liter bag. For example: a hypersensitive response to a short period of hypoxia measured by increase of respiration or heart rate may provide an indication for therapy. Ablation or significant reduction of such response could be indicative of a successful procedure. Also, electrical stimulation, drugs and chemicals (e.g., dopamine, lidocane) exist that can block or excite a carotid body when applied locally or intravenously.

The location and baseline function of the desired area of therapy (including the carotid and aortic chemoreceptors and baroreceptors and corresponding nerves) may be determined prior to therapy by application of stimuli to the carotid body or other organs that would result in an expected change in a physiological or clinical event such as an increase or decrease in SNS activity, heart rate or blood pressure. These stimuli may also be applied after the therapy to determine the effect of the therapy or to indicate the need for repeated application of therapy to achieve the desired physiological or clinical effect(s). The stimuli can be either electrical or chemical in nature and can be delivered via the same or another catheter or can be delivered separately (such as injection of a substance through a peripheral IV to affect the CBC that would be expected to cause a predicted physiological or clinical effect).

A baseline stimulation test may be performed to select patients that may benefit from a carotid body modulation procedure. For example, patients with a high peripheral chemosensitivity gain (e.g., greater than or equal to about two standard deviations above an age matched general population chemosensitivity, or alternatively above a threshold peripheral chemosensitivity to hypoxia of 0.5 or 0.7 ml/min % O2) may be selected for a carotid body modulation procedure. A prospective patient suffering from a cardiac, metabolic, or pulmonary disease (e.g., hypertension, CHF, diabetes) may be selected. The patient may then be tested to assess a baseline peripheral chemoreceptor sensitivity (e.g., minute ventilation, tidal volume, ventilator rate, heart rate, or other response to hypoxic or hypercapnic stimulus). Baseline peripheral chemosensitivity may be assessed using tests known in the art which involve inhalation of a gas mixture having reduced O₂ content (e.g., pure nitrogen, CO₂, helium, or breathable gas mixture with reduced amounts of O₂ and increased amounts of CO₂) or rebreathing of gas into a bag. Concurrently, the patient's minute ventilation or initial sympathetically mediated physiologic parameter such as minute ventilation or HR may be measured and compared to the O₂ level in the gas mixture. Tests like this may elucidate indices called chemoreceptor setpoint and gain. These indices are indicative of chemoreceptor sensitivity. If the patient's chemosensitivity is not assessed to be high (e.g., less than about two standard deviations of an age matched general population chemosensitivity, or other relevant numeric threshold) then the patient may not be a suitable candidate for a carotid body modulation procedure. Conversely, a patient with chemoreceptor hypersensitivity (e.g., greater than or equal to about two standard deviations above normal) may proceed to have a carotid body modulation procedure. Following a carotid body modulation procedure the patient's chemosensitivity may optionally be tested again and compared to the results of the baseline test. The second test or the comparison of the second test to the baseline test may provide an indication of treatment success or suggest further intervention such as possible adjustment of drug therapy, repeating the carotid body modulation procedure with adjusted parameters or location, or performing another carotid body modulation procedure on a second carotid body if the first procedure only targeted one carotid body. It may be expected that a patient having chemoreceptor hypersensitivity or hyperactivity may return to about a normal sensitivity or activity following a successful carotid body modulation procedure.

In an alternative protocol for selecting a patient for a carotid body modulation, patients with high peripheral chemosensitivity or carotid body activity (e.g., ≧about 2 standard deviations above normal) alone or in combination with other clinical and physiologic parameters may be particularly good candidates for carotid body modulation therapy if they further respond positively to temporary blocking of carotid body activity. A prospective patient suffering from a cardiac, metabolic, or pulmonary disease may be selected to be tested to assess the baseline peripheral chemoreceptor sensitivity. A patient without high chemosensitivity may not be a plausible candidate for a carotid body modulation procedure. A patient with a high chemosensitivity may be given a further assessment that temporarily blocks a carotid body chemoreflex. For example a temporary block may be done chemically, for example using a chemical such as intravascular dopamine or dopamine-like substances, intravascular alpha-2 adrenergic agonists, oxygen, in general alkalinity, or local or topical application of atropine externally to the carotid body. A patient having a negative response to the temporary carotid body block test (e.g., sympathetic activity index such as respiration, HR, heart rate variability, MSNA, vasculature resistance, etc. is not significantly altered) may be a less plausible candidate for a carotid body modulation procedure. Conversely, a patient with a positive response to the temporary carotid body block test (e.g., respiration or index of sympathetic activity is altered significantly) may be a more plausible candidate for a carotid body modulation procedure.

There are a number of potential ways to conduct a temporary carotid body block test. Hyperoxia (e.g., higher than normal levels of PO₂) for example, is known to partially block (about a 50%) or reduce afferent sympathetic response of the carotid body. Thus, if a patient's sympathetic activity indexes (e.g., respiration, HR, HRV, MSNA) are reduced by hyperoxia (e.g., inhalation of higher than normal levels of O₂) for 3-5 minutes, the patient may be a particularly plausible candidate for carotid body modulation therapy. A sympathetic response to hyperoxia may be achieved by monitoring minute ventilation (e.g., reduction of more than 20-30% may indicate that a patient has carotid body hyperactivity). To evoke a carotid body response, or compare it to carotid body response in normoxic conditions, CO₂ above 3-4% may be mixed into the gas inspired by the patient (nitrogen content will be reduced) or another pharmacological agent can be used to invoke a carotid body response to a change of CO₂, pH or glucose concentration. Alternatively, “withdrawal of hypoxic drive” to rest state respiration in response to breathing a high concentration O₂ gas mix may be used for a simpler test.

An alternative temporary carotid body block test involves administering a sub-anesthetic amount of anesthetic gas halothane, which is known to temporarily suppress carotid body activity. Furthermore, there are injectable substances such as dopamine that are known to reversibly inhibit the carotid body. However, any substance, whether inhaled, injected or delivered by another manner to the carotid body that affects carotid body function in the desired fashion may be used.

Another alternative temporary carotid body block test involves application of cryogenic energy to a carotid body (i.e., removal of heat). For example, a carotid body or its nerves may be cooled to a temperature range between about −15° C. to 0° C. to temporarily reduce nerve activity or blood flow to and from a carotid body thus reducing or inhibiting carotid body activity.

An alternative method of assessing a temporary carotid body block test may involve measuring pulse pressure. Noninvasive pulse pressure devices such as Nexfin (made by BMEYE, based in Amsterdam, The Netherlands) can be used to track beat-to-beat changes in peripheral vascular resistance. Patients with hypertension or CHF may be sensitive to temporary carotid body blocking with oxygen or injection of a blocking drug. The peripheral vascular resistance of such patients may be expected to reduce substantially in response to carotid body blocking. Such patients may be good candidates for carotid body modulation therapy.

Yet another index that may be used to assess if a patient may be a good candidate for carotid body modulation therapy is increase of baroreflex, or baroreceptor sensitivity, in response to carotid body blocking. It is known that hyperactive chemosensitivity suppresses baroreflex. If carotid body activity is temporarily reduced the carotid sinus baroreflex (baroreflex sensitivity (BRS) or baroreflex gain) may be expected to increase. Baroreflex contributes a beneficial parasympathetic component to autonomic drive. Depressed BRS is often associated with an increased incidence of death and malignant ventricular arrhythmias. Baroreflex is measurable using standard non-invasive methods. One example is spectral analysis of RR interval of ECG and systolic blood pressure variability in both the high- and low-frequency bands. An increase of baroreflex gain in response to temporary blockade of carotid body can be a good indication for permanent therapy. Baroreflex sensitivity can also be measured by heart rate response to a transient rise in blood pressure induced by injection of phenylephrine.

An alternative method involves using an index of glucose tolerance to select patients and determine the results of carotid body blocking or removal in diabetic patients. There is evidence that carotid body hyperactivity contributes to progression and severity of metabolic disease.

In general, a beneficial response can be seen as an increase of parasympathetic or decrease of sympathetic tone in the overall autonomic balance. For example, Power Spectral Density (PSD) curves of respiration or HR can be calculated using nonparametric Fast Fourier Transform algorithm (FFT). FFT parameters can be set to 256-64k buffer size, Hamming window, 50% overlap, 0 to 0.5 or 0.1 to 1.0 Hz range. HR and respiratory signals can be analyzed for the same periods of time corresponding to (1) normal unblocked carotid body breathing and (2) breathing with blocked carotid body.

Power can be calculated for three bands: the very low frequency (VLF) between 0 and 0.04 Hz, the low frequency band (LF) between 0.04-0.15 Hz and the high frequency band (HF) between 0.15-0.4 Hz. Cumulative spectral power in LF and HF bands may also be calculated; normalized to total power between 0.04 and 0.4 Hz (TF=HF+LF) and expressed as % of total. Natural breathing rate of CHF patient, for example, can be rather high, in the 0.3-0.4 Hz range.

The VLF band may be assumed to reflect periodic breathing frequency (typically 0.016 Hz) that can be present in CHF patients. It can be excluded from the HF/LF power ratio calculations.

The powers of the LF and HF oscillations characterizing heart rate variability (HRV) appear to reflect, in their reciprocal relationship, changes in the state of the sympathovagal (sympathetic to parasympathetic) balance occurring during numerous physiological and pathophysiological conditions. Thus, increase of HF contribution in particular can be considered a positive response to carotid body blocking.

Another alternative method of assessing carotid body activity comprises nuclear medicine scanning, for example with ocretide, somatostatin analogues, or other substances produced or bound by the carotid body.

Furthermore, artificially increasing blood flow may reduce carotid body activation. Conversely artificially reducing blood flow may stimulate carotid body activation. This may be achieved with drugs known in the art to alter blood flow.

There is a considerable amount of scientific evidence to demonstrate that hypertrophy of a carotid body often accompanies disease. A hypertrophied (i.e. enlarged) carotid body may further contribute to the disease. Thus identification of patients with enlarged carotid bodies may be instrumental in determining candidates for therapy. Imaging of a carotid body may be accomplished by angiography performed with radiographic, computer tomography, or magnetic resonance imaging.

It should be understood that the available measurements are not limited to those described above. It may be possible to use any single or a combination of measurements that reflect any clinical or physiological parameter effected or changed by either increases or decreases in carotid body function to evaluate the baseline state, or change in state, of a patient's chemosensitivity.

There is a considerable amount of scientific evidence to demonstrate that hypertrophy of a carotid body often accompanies disease. A hypertrophied or enlarged carotid body may further contribute to the disease. Thus identification of patients with enlarged carotid bodies may be instrumental in determining candidates for therapy.

Further, it is possible that although patients do not meet a preselected clinical or physiological definition of high peripheral chemosensitivity (e.g., greater than or equal to about two standard deviations above normal), administration of a substance that suppresses peripheral chemosensitivity may be an alternative method of identifying a patient who is a candidate for the proposed therapy. These patients may have a different physiology or co-morbid disease state that, in concert with a higher than normal peripheral chemosensitivity (e.g., greater than or equal to normal and less than or equal to about 2 standard deviations above normal), may still allow the patient to benefit from carotid body modulation. The proposed therapy may be at least in part based on an objective that carotid body modulation will result in a clinically significant or clinically beneficial change in the patient's physiological or clinical course. It is reasonable to believe that if the desired clinical or physiological changes occur even in the absence of meeting the predefined screening criteria, then therapy could be performed.

While the invention has been described in connection with what is presently considered to be the best mode, it is to be understood that the invention is not to be limited to the disclosed embodiment(s). The invention covers various modifications and equivalent arrangements included within the spirit and scope of the appended claims.

### Physiology:

Ablation of a target ablation site (e.g., peripheral chemoreceptor, carotid body) via an endovascular approach in patients having sympathetically mediated disease and augmented chemoreflex (e.g., high afferent nerve signaling from a carotid body to the central nervous system as in some cases indicated by high peripheral chemosensitivity) has been conceived to reduce peripheral chemosensitivity and reduce afferent signaling from peripheral chemoreceptors to the central nervous system. The expected reduction of chemoreflex activity and sensitivity to hypoxia and other stimuli such as blood flow, blood CO₂, glucose concentration or blood pH can directly reduce afferent signals from chemoreceptors and produce at least one beneficial effect such as the reduction of central sympathetic activation, reduction of the sensation of breathlessness (dyspnea), vasodilation, increase of exercise capacity, reduction of blood pressure, reduction of sodium and water retention, redistribution of blood volume to skeletal muscle, reduction of insulin resistance, reduction of hyperventilation, reduction of tachypnea, reduction of hypocapnia, increase of baroreflex and barosensitivity of baroreceptors, increase of vagal tone, or improve symptoms of a sympathetically mediated disease and may ultimately slow down the disease progression and extend life. It is understood that a sympathetically mediated disease that may be treated with carotid body modulation may comprise elevated sympathetic tone, an elevated sympathetic/parasympathetic activity ratio, autonomic imbalance primarily attributable to central sympathetic tone being abnormally or undesirably high, or heightened sympathetic tone at least partially attributable to afferent excitation traceable to hypersensitivity or hyperactivity of a peripheral chemoreceptor (e.g., carotid body). In some important clinical cases where baseline hypocapnia or tachypnea is present, reduction of hyperventilation and breathing rate may be expected. It is understood that hyperventilation in the context herein means respiration in excess of metabolic needs on the individual that generally leads to slight but significant hypocapnia (blood CO₂ partial pressure below normal of approximately 40 mmHg, for example in the range of 33 to 38 mmHg).

Patients having CHF or hypertension concurrent with heightened peripheral chemoreflex activity and sensitivity often react as if their system was hypercapnic even if it is not. The reaction is to hyperventilate, a maladaptive attempt to rid the system of CO₂, thus overcompensating and creating a hypocapnic and alkalotic system. Some researchers attribute this hypersensitivity/hyperactivity of the carotid body to the direct effect of catecholamines, hormones circulating in excessive quantities in the blood stream of CHF patients. The procedure may be particularly useful to treat such patients who are hypocapnic and possibly alkalotic resulting from high tonic output from carotid bodies. Such patients are particularly predisposed to periodic breathing and central apnea hypopnea type events that cause arousal, disrupt sleep, cause intermittent hypoxia and are by themselves detrimental and difficult to treat.

It is appreciated that periodic breathing of Cheyne Stokes pattern occurs in patients during sleep, exercise and even at rest as a combination of central hypersensitivity to CO₂, peripheral chemosensitivity to O₂ and CO₂ and prolonged circulatory delay. All these parameters are often present in CHF patients that are at high risk of death. Thus, patients with hypocapnea, CHF, high chemosensitivity and prolonged circulatory delay, and specifically ones that exhibit periodic breathing at rest or during exercise or induced by hypoxia are likely beneficiaries of the proposed therapy.

Hyperventilation is defined as breathing in excess of a person's metabolic need at a given time and level of activity. Hyperventilation is more specifically defined as minute ventilation in excess of that needed to remove CO₂ from blood in order to maintain blood CO₂ in the normal range (e.g., around 40 mmHg partial pressure). For example, patients with arterial blood PCO₂ in the range of 32-37 mmHg can be considered hypocapnic and in hyperventilation.

For the purpose of this disclosure hyperventilation is equivalent to abnormally low levels of carbon dioxide in the blood (e.g., hypocapnia, hypocapnea, or hypocarbia) caused by overbreathing. Hyperventilation is the opposite of hypoventilation (e.g., underventilation) that often occurs in patients with lung disease and results in high levels of carbon dioxide in the blood (e.g., hypercapnia or hypercarbia).

A low partial pressure of carbon dioxide in the blood causes alkalosis, because CO2 is acidic in solution and reduced CO2 makes blood pH more basic, leading to lowered plasma calcium ions and nerve and muscle excitability. This condition is undesirable in cardiac patients since it can increase probability of cardiac arrhythmias.

Alkalemia may be defined as abnormal alkalinity, or increased pH of the blood. Respiratory alkalosis is a state due to excess loss of carbon dioxide from the body, usually as a result of hyperventilation. Compensated alkalosis is a form in which compensatory mechanisms have returned the pH toward normal. For example, compensation can be achieved by increased excretion of bicarbonate by the kidneys.

Compensated alkalosis at rest can become uncompensated during exercise or as a result of other changes of metabolic balance. Thus the invented method is applicable to treatment of both uncompensated and compensated respiratory alkalosis.

Tachypnea means rapid breathing. For the purpose of this disclosure a breathing rate of about 6 to 16 breaths per minute at rest is considered normal but there is a known benefit to lower rate of breathing in cardiac patients. Reduction of tachypnea can be expected to reduce respiratory dead space, increase breathing efficiency, and increase parasympathetic tone.

Therapy Example: Role of Chemoreflex and Central Sympathetic Nerve Activity in CHF

Chronic elevation in sympathetic nerve activity (SNA) is associated with the development and progression of certain types of hypertension and contributes to the progression of congestive heart failure (CHF). It is also known that sympathetic excitatory cardiac, somatic, and central/peripheral chemoreceptor reflexes are abnormally enhanced in CHF and hypertension (Ponikowski, 2011 and Giannoni, 2008 and 2009).

Arterial chemoreceptors serve an important regulatory role in the control of alveolar ventilation. They also exert a powerful influence on cardiovascular function.

Delivery of Oxygen (O₂) and removal of Carbon Dioxide (CO₂) in the human body is regulated by two control systems, behavioral control and metabolic control. The metabolic ventilatory control system drives our breathing at rest and ensures optimal cellular homeostasis with respect to pH, partial pressure of carbon dioxide (PCO₂), and partial pressure of oxygen (PO₂). Metabolic control uses two sets of chemoreceptors that provide a fine-tuning function: the central chemoreceptors located in the ventral medulla of the brain and the peripheral chemoreceptors such as the aortic chemoreceptors and the carotid body chemoreceptors. The carotid body, a small, ovoid-shaped (often described as a grain of rice), and highly vascularized organ is situated in or near the carotid bifurcation, where the common carotid artery branches in to an internal carotid artery (IC) and external carotid artery (EC). The central chemoreceptors are sensitive to hypercapnia (high PCO₂), and the peripheral chemoreceptors are sensitive to hypercapnia and hypoxia (low blood PO₂). Under normal conditions activation of the sensors by their respective stimuli results in quick ventilatory responses aimed at the restoration of cellular homeostasis.

As early as 1868, Pflüger recognized that hypoxia stimulated ventilation, which spurred a search for the location of oxygen-sensitive receptors both within the brain and at various sites in the peripheral circulation. When Corneille Heymans and his colleagues observed that ventilation increased when the oxygen content of the blood flowing through the bifurcation of the common carotid artery was reduced (winning him the Nobel Prize in 1938), the search for the oxygen chemosensor responsible for the ventilatory response to hypoxia was largely considered accomplished.

The persistence of stimulatory effects of hypoxia in the absence (after surgical removal) of the carotid chemoreceptors (e.g., the carotid bodies) led other investigators, among them Julius Comroe, to ascribe hypoxic chemosensitivity to other sites, including both peripheral sites (e.g., aortic bodies) and central brain sites (e.g., hypothalamus, pons and rostral ventrolateral medulla). The aortic chemoreceptor, located in the aortic body, may also be an important chemoreceptor in humans with significant influence on vascular tone and cardiac function.

Carotid Body Chemoreflex:

The carotid body is a small cluster of chemoreceptors (also known as glomus cells) and supporting cells located near, and in most cases directly at, the medial side of the bifurcation (fork) of the carotid artery, which runs along both sides of the throat.

These organs act as sensors detecting different chemical stimuli from arterial blood and triggering an action potential in the afferent fibers that communicate this information to the Central Nervous System (CNS). In response, the CNS activates reflexes that control heart rate (HR), renal function and peripheral blood circulation to maintain the desired homeostasis of blood gases, O₂ and CO₂, and blood pH. This closed loop control function that involves blood gas chemoreceptors is known as the carotid body chemoreflex (CBC). The carotid body chemoreflex is integrated in the CNS with the carotid sinus baroreflex (CSB) that maintains arterial blood pressure. In a healthy organism these two reflexes maintain blood pressure and blood gases within a narrow physiologic range. Chemosensors and barosensors in the aortic arch contribute redundancy and fine-tuning function to the closed loop chemoreflex and baroreflex. In addition to sensing blood gasses, the carotid body is now understood to be sensitive to blood flow and velocity, blood Ph and glucose concentration. Thus it is understood that in conditions such as hypertension, CHF, insulin resistance, diabetes and other metabolic derangements afferent signaling of carotid body nerves may be elevated. Carotid body hyperactivity may be present even in the absence of detectable hypersensitivity to hypoxia and hypercapnia that are traditionally used to index carotid body function. The purpose of the proposed therapy is therefore to remove or reduce afferent neural signals from a carotid body and reduce carotid body contribution to central sympathetic tone.

The carotid sinus baroreflex is accomplished by negative feedback systems incorporating pressure sensors (e.g., baroreceptors) that sense the arterial pressure. Baroreceptors also exist in other places, such as the aorta and coronary arteries. Important arterial baroreceptors are located in the carotid sinus, a slight dilatation of the internal carotid artery at its origin from the common carotid. The carotid sinus baroreceptors are close to but anatomically separate from the carotid body. Baroreceptors respond to stretching of the arterial wall and communicate blood pressure information to CNS. Baroreceptors are distributed in the arterial walls of the carotid sinus while the chemoreceptors (glomus cells) are clustered inside the carotid body. This makes the selective reduction of chemoreflex described in this application possible while substantially sparing the baroreflex.

The carotid body exhibits great sensitivity to hypoxia (low threshold and high gain). In chronic Congestive Heart Failure (CHF), the sympathetic nervous system activation that is directed to attenuate systemic hypoperfusion at the initial phases of CHF may ultimately exacerbate the progression of cardiac dysfunction that subsequently increases the extra-cardiac abnormalities, a positive feedback cycle of progressive deterioration, a vicious cycle with ominous consequences. It was thought that much of the increase in the sympathetic nerve activity (SNA) in CHF was based on an increase of sympathetic flow at a level of the CNS and on the depression of arterial baroreflex function. In the past several years, it has been demonstrated that an increase in the activity and sensitivity of peripheral chemoreceptors (heightened chemoreflex function) also plays an important role in the enhanced SNA that occurs in CHF.

Role of Altered Chemoreflex in CHF:

As often happens in chronic disease states, chemoreflexes that are dedicated under normal conditions to maintaining homeostasis and correcting hypoxia contribute to increase the sympathetic tone in patients with CHF, even under normoxic conditions. The understanding of how abnormally enhanced sensitivity of the peripheral chemosensors, particularly the carotid body, contributes to the tonic elevation in SNA in patients with CHF has come from several studies in animals. According to one theory, the local angiotensin receptor system plays a fundamental role in the enhanced carotid body chemoreceptor sensitivity in CHF. In addition, evidence in both CHF patients and animal models of CHF has clearly established that the carotid body chemoreflex is often hypersensitive in CHF patients and contributes to the tonic elevation in sympathetic function. This derangement derives from altered function at the level of both the afferent and central pathways of the reflex arc. The mechanisms responsible for elevated afferent activity from the carotid body in CHF are not yet fully understood.

Regardless of the exact mechanism behind the carotid body hypersensitivity, the chronic sympathetic activation driven from the carotid body and other autonomic pathways leads to further deterioration of cardiac function in a positive feedback cycle. As CHF ensues, the increasing severity of cardiac dysfunction leads to progressive escalation of these alterations in carotid body chemoreflex function to further elevate sympathetic activity and cardiac deterioration. The trigger or causative factors that occur in the development of CHF that sets this cascade of events in motion and the time course over which they occur remain obscure. Ultimately, however, causative factors are tied to the cardiac pump failure and reduced cardiac output. According to one theory, within the carotid body, a progressive and chronic reduction in blood flow may be the key to initiating the maladaptive changes that occur in carotid body chemoreflex function in CHF.

There is sufficient evidence that there is increased peripheral and central chemoreflex sensitivity in heart failure, which is likely to be correlated with the severity of the disease. There is also some evidence that the central chemoreflex is modulated by the peripheral chemoreflex. According to current theories, the carotid body is the predominant contributor to the peripheral chemoreflex in humans; the aortic body having a minor contribution.

Although the mechanisms responsible for altered central chemoreflex sensitivity remain obscure, the enhanced peripheral chemoreflex sensitivity can be linked to a depression of nitric oxide production in the carotid body affecting afferent sensitivity, and an elevation of central angiotensin II affecting central integration of chemoreceptor input. The enhanced chemoreflex may be responsible, in part, for the enhanced ventilatory response to exercise, dyspnea, Cheyne-Stokes breathing, and sympathetic activation observed in chronic heart failure patients. The enhanced chemoreflex may be also responsible for hyperventilation and tachypnea (e.g., fast breathing) at rest and exercise, periodic breathing during exercise, rest and sleep, hypocapnia, vasoconstriction, reduced peripheral organ perfusion and hypertension.

Dyspnea:

Shortness of breath, or dyspnea, is a feeling of difficult or labored breathing that is out of proportion to the patient's level of physical activity. It is a symptom of a variety of different diseases or disorders and may be either acute or chronic. Dyspnea is the most common complaint of patients with cardiopulmonary diseases.

Dyspnea is believed to result from complex interactions between neural signaling, the mechanics of breathing, and the related response of the central nervous system. A specific area has been identified in the mid-brain that may influence the perception of breathing difficulties.

The experience of dyspnea depends on its severity and underlying causes. The feeling itself results from a combination of impulses relayed to the brain from nerve endings in the lungs, rib cage, chest muscles, or diaphragm, combined with the perception and interpretation of the sensation by the patient. In some cases, the patient's sensation of breathlessness is intensified by anxiety about its cause. Patients describe dyspnea variously as unpleasant shortness of breath, a feeling of increased effort or tiredness in moving the chest muscles, a panicky feeling of being smothered, or a sense of tightness or cramping in the chest wall.

The four generally accepted categories of dyspnea are based on its causes: cardiac, pulmonary, mixed cardiac or pulmonary, and non-cardiac or non-pulmonary. The most common heart and lung diseases that produce dyspnea are asthma, pneumonia, COPD, and myocardial ischemia or heart attack (myocardial infarction). Foreign body inhalation, toxic damage to the airway, pulmonary embolism, congestive heart failure (CHF), anxiety with hyperventilation (panic disorder), anemia, and physical deconditioning because of sedentary lifestyle or obesity can produce dyspnea. In most cases, dyspnea occurs with exacerbation of the underlying disease. Dyspnea also can result from weakness or injury to the chest wall or chest muscles, decreased lung elasticity, obstruction of the airway, increased oxygen demand, or poor pumping action of the heart that results in increased pressure and fluid in the lungs, such as in CHF.

Acute dyspnea with sudden onset is a frequent cause of emergency room visits. Most cases of acute dyspnea involve pulmonary (lung and breathing) disorders, cardiovascular disease, or chest trauma. Sudden onset of dyspnea (acute dyspnea) is most typically associated with narrowing of the airways or airflow obstruction (bronchospasm), blockage of one of the arteries of the lung (pulmonary embolism), acute heart failure or myocardial infarction, pneumonia, or panic disorder.

Chronic dyspnea is different. Long-standing dyspnea (chronic dyspnea) is most often a manifestation of chronic or progressive diseases of the lung or heart, such as COPD, which includes chronic bronchitis and emphysema. The treatment of chronic dyspnea depends on the underlying disorder. Asthma can often be managed with a combination of medications to reduce airway spasms and removal of allergens from the patient's environment. COPD requires medication, lifestyle changes, and long-term physical rehabilitation. Anxiety disorders are usually treated with a combination of medication and psychotherapy.

Although the exact mechanism of dyspnea in different disease states is debated, there is no doubt that the CBC plays some role in most manifestations of this symptom. Dyspnea seems to occur most commonly when afferent input from peripheral receptors is enhanced or when cortical perception of respiratory work is excessive.

Surgical Removal of the Glomus and Resection of Carotid Body Nerves:

A surgical treatment for asthma, removal of the carotid body or glomus (glomectomy), was described by Japanese surgeon Komei Nakayama in 1940s. According to Nakayama in his study of 4,000 patients with asthma, approximately 80% were cured or improved six months after surgery and 58% allegedly maintained good results after five years. Komei Nakayama performed most of his surgeries while at the Chiba University during World War II. Later in the 1950's, a U.S. surgeon, Dr. Overholt, performed the Nakayama operation on 160 U.S. patients. He felt it necessary to remove both carotid bodies in only three cases. He reported that some patients feel relief the instant when the carotid body is removed, or even earlier, when it is inactivated by an injection of procaine (Novocain).

Overholt, in his paper Glomectomy for Asthma published in Chest in 1961, described surgical glomectomy the following way: “A two-inch incision is placed in a crease line in the neck, one-third of the distance between the angle of the mandible and clavicle. The platysma muscle is divided and the sternocleidomastoid retracted laterally. The dissection is carried down to the carotid sheath exposing the bifurcation. The superior thyroid artery is ligated and divided near its take-off in order to facilitate rotation of the carotid bulb and expose the medial aspect of the bifurcation. The carotid body is about the size of a grain of rice and is hidden within the adventitia of the vessel and is of the same color. The perivascular adventitia is removed from one centimeter above to one centimeter below the bifurcation. This severs connections of the nerve plexus, which surrounds the carotid body. The dissection of the adventitia is necessary in order to locate and identify the body. It is usually located exactly at the point of bifurcation on its medial aspect. Rarely, it may be found either in the center of the crotch or on the lateral wall. The small artery entering the carotid body is clamped, divided, and ligated. The upper stalk of tissue above the carotid body is then clamped, divided, and ligated.”

In January 1965, the New England Journal of Medicine published a report of 15 cases in which there had been unilateral removal of the cervical glomus (carotid body) for the treatment of bronchial asthma, with no objective beneficial effect. This effectively stopped the practice of glomectomy to treat asthma in the U.S.

Winter developed a technique for separating nerves that contribute to the carotid sinus nerves into two bundles, carotid sinus (baroreflex) and carotid body (chemoreflex), and selectively cutting out the latter. The Winter technique is based on his discovery that carotid sinus (baroreflex) nerves are predominantly on the lateral side of the carotid bifurcation and carotid body (chemoreflex) nerves are predominantly on the medial side.

Neuromodulation of the Carotid Body Chemoreflex:

Hlavaka in U.S. Patent Application Publication 2010/0070004 filed Aug. 7, 2009, describes implanting an electrical stimulator to apply electrical signals, which block or inhibit chemoreceptor signals in a patient suffering dyspnea. Hlavaka teaches that “some patients may benefit from the ability to reactivate or modulate chemoreceptor functioning.” Hlavaka focuses on neuromodulation of the chemoreflex by selectively blocking conduction of nerves that connect the carotid body to the CNS. Hlavaka describes a traditional approach of neuromodulation with an implantable electric pulse generator that does not modify or alter tissue of the carotid body or chemoreceptors.

The central chemoreceptors are located in the brain and are difficult to access. The peripheral chemoreflex is modulated primarily by carotid bodies that are more accessible. Previous clinical practice had very limited clinical success with the surgical removal of carotid bodies to treat asthma in 1940s and 1960s.

1.-41. (canceled)

42. A method for ablating carotid body function in a patient comprising:
a. inserting a vascular access sheath into a superficial temporal artery;
b. inserting an ablation catheter through the sheath, with the ablation catheter comprising a catheter shaft, an ablation element disposed in the vicinity of the distal end of the catheter shaft and in communication with an ablation energy source;
c. positioning the ablation element within an external carotid artery adjacent to the target site;
d. applying ablation energy with the ablation element at an energy level and for a duration sufficient to substantially ablate carotid body function.

43. The method of claim 42 wherein the ablation element is a mono-polar RF ablation electrode, and the ablation energy source is an RF generator and a connection is made between the mono-polar RF ablation electrode and a pole of the RF generator and wherein an indifferent electrode is placed upon or within the body of the patient and connected to the second pole of the RF generator.

44. (canceled)

45. The method of claim 43 wherein the indifferent electrode is placed upon, or within the patient's body at a location that positions the target site between the ablation electrode and indifferent electrode.

46. The method of claim 45 wherein the indifferent electrode is placed at a location selected from a group consisting of within an internal jugular vein, within muscle of the patient's neck, and on the skin of the patient's neck.

47.-48. (canceled)

49. The method of claim 43 wherein the indifferent electrode comprises a means for preventing thermal injury to its surroundings.

50.-51. (canceled)

52. The method of claim 42 wherein the ablation element is a substantially cylindrical mono-polar RF electrode with a means for substantial surface irrigation by an ionic liquid.

53. (canceled)

54. The method of claim 42 wherein the ablation element is a substantially lateral mono-polar RF electrode configured to apply RF energy to an intercarotid septum while substantially avoiding application of RF energy to arterial blood.

55. The method of claim 42 wherein the ablation element comprises a hollow cylindrical structure with at least one lateral fenestration, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface.

56. The method of claim 42 wherein the ablation element is a pair of bi-polar RF electrodes mounted in tandem.

57. The method of claim 42 wherein the ablation element is a substantially lateral pair of bi-polar RF electrodes mounted in tandem configured to apply RF energy to the wall of a carotid artery and substantially avoiding applying RF energy to arterial blood.

58. (canceled)

59. The method of claim 42 wherein the ablation element comprises a piezo-electric element configured for laterally directed emission of ultrasonic energy.

60.-61. (canceled)

62. The method of claim 42 wherein the ablation element comprising at least one RF electrode mounted on the surface of an expandable structure.

63.-64. (canceled)

65. The method of claim 42 comprising a means for pressing the ablation element against the wall of a carotid artery comprising a mechanism selected from a list consisting of a push wire, a pull wire, an inflatable balloon, and an expandable cage, a pull wire configured for deflecting the distal end of the catheter in a lateral direction by means of an actuator mounted in the vicinity of the proximal end of the catheter shaft.

66.-68. (canceled)

69. The method of claim 42 further comprises the step of determining carotid body function.

70.-79. (canceled)

80. A method for ablating carotid body function in a patient comprising:
a. inserting a vascular access sheath into a superficial temporal artery; inserting an ablation catheter through the sheath, with the ablation catheter comprising a catheter shaft comprising a flexible elongated structure with a distal end, a proximal end, an articulateable RF ablation electrode, and a second RF electrode, wherein each of the electrodes is in communication with an opposing pole of an RF generator;
b. positioning the articulate able RF electrode against the wall of the internal carotid artery adjacent to the target site, positioning the second electrode against the wall of the external carotid artery adjacent to the target site;
c. applying RF energy with the electrodes at an energy level and for a duration sufficient to substantially ablate carotid function.

81. A device for trans-temporal artery carotid body modulation comprising:
a. a catheter shaft comprising a flexible elongated structure with a distal end, and a proximal end, a lumen configured to house a deployable and retractable RF ablation electrode in the vicinity of the distal end, the RF electrode in communication with an RF energy source; and
b. a slidable structure comprising a pre-formed curve disposed between the RF electrode and an actuator in the vicinity of the proximal end configured for deploying and retracting the electrode.

82. The device of claim 81 wherein a second RF electrode is disposed on the surface of the catheter shaft in the vicinity of the distal end.

83. The device of claim 82 wherein the second RF electrode is connectable to the second pole of the RF energy source.

84. The device of claim 81 wherein the catheter shaft has a caliber between approximately 3 French and 6 French and a length between approximately 10 centimeters and 25 centimeters.

85. A device for trans-temporal artery carotid body modulation comprising:
a. a catheter shaft comprising a flexible elongated structure with a distal end, and a proximal end;
b. a first RF electrode disposed at the distal end;
c. a second RF electrode disposed proximal to the first electrode;
d. a user articulateable catheter segment between the first electrode and the second electrode,
whereby, the catheter segment is configured to be actuated to vary the spatial relationship between the two electrodes from a substantially axial alignment to a substantially lateral opposition.
